(19) United States
(12) Patent Application Publication
(10) Pub. No.: US 2020/0381087 A1
(43) Pub. Date: Dec. 3, 2020

(54) SYSTEMS AND METHODS OF CLINICAL TRIAL EVALUATION

(71) Applicant: Tempus Labs, Chicago, IL (US)

(72) Inventors: Jonathan Ozeran, Chicago, IL (US); Christopher Paul Tava, Hackettstown, NJ (US); Amy Gordon Franzen, Chicago, IL (US); Kevin Ritt, Chicago, IL (US); Marc Ackermann, Chicago, IL (US)

(21) Appl. No.: 16/889,779

(22) Filed: Jun. 1, 2020

Related U.S. Application Data
(60) Provisional application No. 62/855,913, filed on May 31, 2019.

(51) Int. Cl. G16H 10/20; G16H 10/60; G16H 10/40; G16H 15/00; G06F 40/279; G06F 16/31; G06K 9/00

(52) U.S. Cl. CPC G16H 10/20; G16H 10/60; G16H 10/40; G06K 2209/01; G06F 40/279; G06F 16/31; G06K 9/00469; G16H 15/00

(57) ABSTRACT

Systems and methods are configured to match a patient to a clinical trial. A method includes receiving text-based criteria for the clinical trial, including a molecular marker. Additionally, the method includes associating at least a portion of the text-based criteria to one or more pre-defined data fields containing molecular marker information. The method further includes comparing a molecular marker of the patient to the one or more pre-defined data fields, and generating a report for a provider. The report is based on the comparison and includes a match indication of the patient to the clinical trial.

### CROSS-REFERENCE TO RELATED APPLICATIONS

This application is based on, claims the benefit of, and claims priority to U.S. Provisional Application No. 62/855,913, filed May 31 2019, which is hereby incorporated by reference herein in its entirety for all purposes.

### STATEMENT REGARDING FEDERALLY SPONSORED RESEARCH

N/A.

### BACKGROUND

The present disclosure relates to systems and methods for facilitating the extraction and analysis of data embedded within clinical trial information and patient records. More particularly, the present disclosure relates to systems and methods for matching patients with clinical trials and validating clinical trial site capabilities.

The present disclosure is described in the context of a system that utilizes an established database of clinical trials (e.g., clinicaltrials.gov, as provided by the U.S. National Library of Medicine). Nevertheless, it should be appreciated that the present disclosure is intended to teach concepts, features, and aspects that can be useful with any information source relating to clinical trials, including, for example, independently documented clinical trials, internally/privately developed clinical trials, a plurality of clinical trial databases, and the like.

Hereafter, unless indicated otherwise, the following terms and phrases will be used in this disclosure as described. The term “provider” will be used to refer to an entity that operates the overall system disclosed herein and, in most cases, will include a company or other entity that runs servers and maintains databases and that employs people with many different skill sets required to construct, maintain and adapt the disclosed system to accommodate new data types, new medical and treatment insights, and other needs. Exemplary provider employees may include principal investigators, clinical researcher administrators, researchers, physicians, nurses, and/or other healthcare providers, researchers, data abstractors, site specialists, data scientists, and many other persons with specialized skill sets.

The term “physician” will be used to refer generally to any health care provider including but not limited to a primary care physician, a medical specialist, a neurologist, a radiologist, a geneticist, and a medical assistant, among others.

The term “data abstractor” will be used to refer to a person that consumes data available in clinical records provided by a physician (such as primary care physician or specialist) to generate normalized and structured data for use by other system specialists, and/or within the system.

The term “clinical trial” will be used to refer to a research study in which human volunteers are assigned to interventions (e.g., a medical product, behavior, or procedure) based on a protocol and are then evaluated for effects on biomedical or health outcomes.

Existing clinical trial databases and systems can be web-based resources that provide patients, providers, physicians, researchers, and the general public with access to information on publicly and privately supported clinical studies. Often, there are a large number of clinical trials being conducted at any given time, and typically the clinical trials relate to a wide range of diseases and conditions. In some instances, clinical trials are performed at or using the resources of multiple sites, such as hospitals, laboratories, and universities. Each site that participates in a given clinical trial must have the proper equipment, protocols, and staff expertise, among other things.

Clinical trial databases and systems receive information on each clinical trial via the submission of data by the principal investigator (PI) or sponsor (or related staff). As an example, the public website clinicaltrials.gov is maintained by the National Library of Medicine (NLM) at the National Institutes of Health (NIH). Most of the records on clinicaltrials.gov describe clinical trials.

The information on clinicaltrials.gov is typically provided and updated by the sponsor (or PI) of the particular clinical trial. Studies and clinical trials are generally submitted (that is, registered) to relevant websites and databases when they begin, and the information may be updated as-needed throughout the study or trial. Studies and clinical trials listed in the database span the United States, as well as over two hundred additional countries. Notably, clinicaltrials.gov and/or other clinical trial databases may not contain information about all the clinical trials conducted in the United States (or globally), because not all studies are currently required by law to be registered. Additionally, trial databases are often not maintained to include the most up-to-date information about the conduct of any particular study.

In general, each clinical trial record (such as on clinicaltrials.gov), presents summary information about a study protocol which can include the disease or condition, the proposed intervention (e.g., the medical product, behavior, or procedure being studied), title, description, and design of the trial, requirements for participation (eligibility criteria), locations where the trial is being conducted (sites), and/or contact information for the sites.

Notably, clinical trial databases and websites often express the clinical trial information using free text (i.e., unstructured data). For example, one trial on clinicaltrials.gov is a Phase I/II clinical trial using the drugs sapacitabine and olaparib. According to the study description, “the FDA (the U.S. Food and Drug Administration) has approved Olaparib as a treatment for metastatic HER2 negative breast cancer with a BRCA mutation. Olaparib is an inhibitor of PARP (poly [adenosine diphosphate-ribose] polymerase), which means that it stops PARP from working. PARP is an enzyme (a type of protein) found in the cells of the body. In normal cells when DNA is damaged, PARP helps to repair the damage. The FDA has not approved Sapacitabine for use in patients including people with this type of cancer. Sapacitabine and drugs of its class have been shown to have antitumor properties in many types of cancer, e.g., leukemia, lung, breast, ovarian, pancreatic and bladder cancer. Sapacitabine may help to stop the growth of some types of cancers. In this research study, the investigators are evaluating the safety and effectiveness of Olaparib in combination with Sapacitabine in BRCA mutant breast cancer.” The trial has fourteen inclusion criteria and twenty exclusion criteria, each described using free text. One inclusion criteria for the clinical trial is “Documented germline mutation in BRCA1 or BRCA2 that is predicted to be deleterious or suspected deleterious (known or predicted to be detrimental/lead to loss of function). Testing may be completed by any CLIA-certified laboratory.” Another inclusion criteria for the clinical trial states that the patient must have “Adequate organ and bone marrow function as defined below:

Hemoglobin >=10 g/dL

Absolute neutrophil count (ANC) >=1.5×109/L

Platelet count >=100×109/L

Total bilirubin <=1.5×institutional upper limit of normal (ULN)

AST(SGOT)ALT (SGPT) <=2.5×institutional ULN, OR

AST(SGOT)ALT (SGPT) <=5×institutional ULN if liver metastases are present

Creatinine Clearance estimated (using the Cockcroft-Gault equation) of >=51 mL/min.”

When described with free text, inclusion criteria requires a physician or other person to review the inclusion criteria compared to a patient's medical record to determine whether the patient is eligible for the study. Some patient health information is in the form of structured data, where health information resides within a fixed field within a record or file, such as a database or a spreadsheet. The free text nature of the inclusion criteria presented by websites such as clinicaltrials.gov does not lend itself to simple matching with structured data, and inclusion criteria that are described on the website require analysis of multiple structured data fields. For example, the inclusion criteria “Documented germline mutation in BRCA1 or BRCA2 that is predicted to be deleterious or suspected deleterious (known or predicted to be detrimental/lead to loss of function). Testing may be completed by any CLIA-certified laboratory” requires analysis of 1) the particular mutation, 2) whether it is germline, 3) whether it is deleterious, predicted to be detrimental, or leads to a loss of function, 4) whether it was tested in a CLIA-certified laboratory. With respect to unstructured clinical trial data, efficiently determining factors such as eligibility criteria for a potential patient participant often becomes unmanageable.

Thus, what is needed is a system that is capable of efficiently capturing all relevant clinical trial and patient data, including disease/condition data, trial eligibility criteria, trial site features and constraints, and/or clinical trial status (recruiting, active, closed, etc.). Further, what is needed is a system capable of structuring that data to optimally drive different system activities including one or more of efficiently matching patients to clinical trials, activating new sites for an existing clinical trial, and updating site information, among other things. In addition, the system should be highly and rapidly adaptable so that it can be modified to absorb new data types and new clinical trial information, as well as to enable development of new user applications and interfaces optimized to specific user activities.

### BRIEF SUMMARY OF THE DISCLOSURE

One implementation of the present disclosure is a method of matching a patient to a clinical trial. The method includes receiving text-based criteria for the clinical trial, including a molecular marker, associating at least a portion of the text-based criteria to one or more pre-defined data fields containing molecular marker information, comparing a molecular marker of the patient to the one or more pre-defined data fields, and generating a report for a provider, the report based on the comparison and including a match indication of the patient to the clinical trial.

In some aspects, the molecular marker can be an RNA sequence.

In some aspects, the molecular marker can be an DNA sequence.

In some aspects, the one or more pre-defined data fields can include inclusion criteria and exclusion criteria.

In some aspects, the method can further include determining that the patient has not received a treatment related to the molecular marker of the patient, and determining that the patient is eligible for at least one candidate clinical trial in response to determining that the patient has not received the treatment.

In some aspects, at least a portion of the text based criteria can be free-text.

Another implementation of the present disclosure is a clinical trial matching system including at least one processor and at least one memory. The system is configured to receive text-based criteria for a clinical trial, including a molecular marker, associate at least a portion of the text-based criteria to one or more pre-defined data fields containing molecular marker information, compare a molecular marker of a patient to the one or more pre-defined data fields, and generate a report for a provider, the report based on the comparison and including a match indication of the patient to the clinical trial.

In some aspects, the molecular marker can be an RNA sequence.

In some aspects, the molecular marker can be a DNA sequence.

In some aspects, the one or more pre-defined data fields can include inclusion criteria and exclusion criteria.

In some aspects, the system can be further configured to determine that the patient has not received a treatment related to the molecular marker of the patient, and determine that the patient is eligible for at least one candidate clinical trial in response to determining that the patient has not received the treatment.

In some aspects, at least a portion of the text based criteria is free-text.

Yet another implementation of the present disclosure is a method of matching a patient to a clinical trial. The method includes receiving health information from an electronic medical record corresponding to the patient, determining data elements within the health information using at least one of an optical character recognition (OCR) method and a natural language processing (NLP) method, comparing the data elements to pre-determined trial criteria, including trial inclusion criteria and trial exclusion criteria, determining at least one matching clinical trial, based on the comparing of the data elements to the predetermined trial criteria, and notifying a practitioner associated with the patient of the at least one matching clinical trial.

In some aspects, the pre-determined trial criteria can be generated based on unstructured text.

In some aspects, the pre-determined trial criteria can be formatted in at least one standardized format in use by a medical institution.

In some aspects, the data elements can include at least one of a clinical feature, a molecular feature, an epigenome feature, a microbiome feature, an organoid feature, or an imaging feature.

In some aspects, the method can further include periodically updating a clinical trial database including the at least one matching clinical trial and at least one non-matching trial.

In some aspects, the notifying the practitioner associated with the patient of the at least one matching clinical trial can include causing a report to be displayed to the practitioner, the report comprising the locations of the at least one matching trial.

A further implementation of the present disclosure is a clinical trial matching system including at least one processor and at least one memory. The system is configured to receive health information from an electronic medical record corresponding to the patient, determine data elements within the health information using at least one of an optical character recognition (OCR) method and a natural language processing (NLP) method, compare the data elements to pre-determined trial criteria, including trial inclusion criteria and trial exclusion criteria, determine at least one matching clinical trial, based on the comparing of the data elements to the predetermined trial criteria, and notify a practitioner associated with the patient of the at least one matching clinical trial.

In some aspects, the pre-determined trial criteria can be generated based on unstructured text.

In some aspects, the pre-determined trial criteria can be formatted in at least one standardized format in use by a medical institution.

In some aspects, the data elements can include at least one of a clinical feature, a molecular feature, an epigenome feature, a microbiome feature, an organoid feature, or an imaging feature.

In some aspects, the system can be further configured to periodically update a clinical trial database comprising the at least one matching clinical trial and at least one non-matching trial.

In some aspects, the notifying the practitioner associated with the patient of the at least one matching clinical trial can include causing a report to be displayed to the practitioner, the report comprising the locations of the at least one matching trial.

To the accomplishment of the foregoing and related ends, the disclosure, then, includes the features hereinafter fully described. The following description and the annexed drawings set forth in detail certain illustrative aspects of the disclosure. However, these aspects are indicative of but a few of the various ways in which the principles of the disclosure can be employed. Other aspects, advantages and novel features of the disclosure will become apparent from the following detailed description of the invention when considered in conjunction with the drawings.

### BRIEF DESCRIPTION OF THE DRAWINGS

FIG. 1 is a block diagram of a data-based healthcare system, according to aspects of the present disclosure;

FIG. 2 is an image of an example graphical user interface (GUI), according to aspects of the present disclosure;

FIG. 3 is an image of an example graphical user interface (GUI), according to aspects of the present disclosure;

FIG. 4 is an image of an example graphical user interface (GUI), according to aspects of the present disclosure;

FIG. 5 is an image of an example graphical user interface (GUI), according to aspects of the present disclosure;

FIG. 6 is an image of an example graphical user interface (GUI), according to aspects of the present disclosure;

FIG. 7 is an image of an example graphical user interface (GUI), according to aspects of the present disclosure;

FIG. 8 is an image of an example graphical user interface (GUI), according to aspects of the present disclosure;

FIG. 9 is an image of an example graphical user interface (GUI), according to aspects of the present disclosure;

FIG. 10 is an image of an example graphical user interface (GUI), according to aspects of the present disclosure;

FIG. 11 is an image of an example graphical user interface (GUI), according to aspects of the present disclosure;

FIG. 12 is an image of an example graphical user interface (GUI), according to aspects of the present disclosure;

FIG. 13 is an image of an example graphical user interface (GUI), according to aspects of the present disclosure;

FIG. 14 is an image of an example graphical user interface (GUI), according to aspects of the present disclosure;

FIG. 15 is an image of an example graphical user interface (GUI), according to aspects of the present disclosure;

FIG. 16 is an image of an example graphical user interface (GUI), according to aspects of the present disclosure;

FIG. 17 is an image of an example graphical user interface (GUI), according to aspects of the present disclosure;

FIG. 18 is an image of an example graphical user interface (GUI), according to aspects of the present disclosure;

FIG. 19 is an image of an example graphical user interface (GUI), according to aspects of the present disclosure;

FIG. 20 is an image of an example graphical user interface (GUI), according to aspects of the present disclosure;

FIG. 21 is an image of an example graphical user interface (GUI), according to aspects of the present disclosure;

FIG. 22 is an image of an example graphical user interface (GUI), according to aspects of the present disclosure;

FIG. 23 is an image of an example graphical user interface (GUI), according to aspects of the present disclosure;

FIG. 24 is an image of an example graphical user interface (GUI), according to aspects of the present disclosure;

FIG. 25A is an image of an example graphical user interface (GUI), according to aspects of the present disclosure;

FIG. 25B is another image of the example GUI of FIG. 25A, according to aspects of the present disclosure;

FIG. 26 is an image of an example graphical user interface (GUI), according to aspects of the present disclosure;

FIG. 27 is an image of an example graphical user interface (GUI), according to aspects of the present disclosure;

FIG. 28A is an image of an example graphical user interface (GUI), according to aspects of the present disclosure;

FIG. 28B is another image of the example GUI of FIG. 28A, according to aspects of the present disclosure;

FIG. 28C is another image of the example GUI of FIG. 28A, according to aspects of the present disclosure;

FIG. 29 is an image of an example graphical user interface (GUI), according to aspects of the present disclosure; and

FIG. 30 is an image of an example graphical user interface (GUI), according to aspects of the present disclosure.

FIG. 31A is an image of an example graphical user interface (GUI), according to aspects of the present disclosure.

FIG. 31B is an image of an example graphical user interface (GUI), according to aspects of the present disclosure.

FIG. 31C is an image of an example graphical user interface (GUI), according to aspects of the present disclosure.

FIG. 31D is an image of an example graphical user interface (GUI), according to aspects of the present disclosure.

FIG. 31E is an image of an example graphical user interface (GUI), according to aspects of the present disclosure.

FIG. 31F is an image of an example graphical user interface (GUI), according to aspects of the present disclosure.

FIG. 31G is an image of an example graphical user interface (GUI), according to aspects of the present disclosure.

FIG. 31H is an image of an example graphical user interface (GUI), according to aspects of the present disclosure.

FIG. 32 is an exemplary flow for mapping clinical trial inclusion and exclusion criteria to a patient.

FIG. 33 is an image of an example graphical user interface (GUI), according to aspects of the present disclosure.

FIG. 34 is an image of an example graphical user interface (GUI), according to aspects of the present disclosure.

FIG. 35 is an image of an example graphical user interface (GUI), according to aspects of the present disclosure.

FIG. 36 is an image of an example graphical user interface (GUI), according to aspects of the present disclosure.

FIG. 37 is an image of an example graphical user interface (GUI), according to aspects of the present disclosure.

FIG. 38 is an image of an example graphical user interface (GUI), according to aspects of the present disclosure.

FIG. 39 is an image of an example graphical user interface (GUI), according to aspects of the present disclosure.

FIG. 40 is an image of an example graphical user interface (GUI), according to aspects of the present disclosure.

FIG. 41 is an image of an example graphical user interface (GUI), according to aspects of the present disclosure.

FIG. 42 is an image of an example graphical user interface (GUI), according to aspects of the present disclosure.

FIG. 43 is an image of an example graphical user interface (GUI), according to aspects of the present disclosure.

FIG. 44 is an image of an example graphical user interface (GUI), according to aspects of the present disclosure.

FIG. 45 is an image of an example graphical user interface (GUI), according to aspects of the present disclosure.

FIG. 46 is an image of an example graphical user interface (GUI), according to aspects of the present disclosure.

FIG. 47 is an image of an example graphical user interface (GUI), according to aspects of the present disclosure.

FIG. 48 is an image of an example graphical user interface (GUI), according to aspects of the present disclosure.

FIG. 49 is an exemplary process for determining patient eligibility for a clinical trial.

FIG. 50 is an exemplary flow for determining whether or not a next-generation sequencing (NGS) report is included in a medical report associated with a patient.

While the present disclosure is susceptible to various modifications and alternative forms, specific embodiments thereof have been shown by way of example in the drawings and are herein described in detail.

### DETAILED DESCRIPTION OF THE DISCLOSURE

The various aspects of the subject invention are now described with reference to the annexed drawings, wherein like reference numerals correspond to similar elements throughout the several views (e.g., “trial description 203” can be similar to “trial description 403”). It should be understood, however, that the drawings and detailed description hereafter relating thereto are not intended to limit the claimed subject matter to the particular form disclosed. Rather, the intention is to cover all modifications, equivalents, and alternatives falling within the spirit and scope of the claimed subject matter.

As used herein, the terms “component,” “system” and the like are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a computer and the computer can be a component. One or more components may reside within a process and/or thread of execution and a component may be localized on one computer and/or distributed between two or more computers or processors.

The word “exemplary” is used herein to mean serving as an example, instance, or illustration. Any aspect or design described herein as “exemplary” is not necessarily to be construed as preferred or advantageous over other aspects or designs.

Furthermore, the disclosed subject matter may be implemented as a system, method, apparatus, or article of manufacture using programming and/or engineering techniques to produce software, firmware, hardware, or any combination thereof to control a computer or processor based device to implement aspects detailed herein. The term “article of manufacture” (or alternatively, “computer program product”) as used herein is intended to encompass a computer program accessible from any computer-readable device, carrier, or media. For example, computer readable media can include but are not limited to magnetic storage devices (such as hard disk, floppy disk, magnetic strips), optical disks (such as compact disk (CD), digital versatile disk (DVD)), smart cards, and flash memory devices (such as card, stick). Additionally it should be appreciated that a carrier wave can be employed to carry computer-readable electronic data such as those used in transmitting and receiving electronic mail or in accessing a network such as the Internet or a local area network (LAN). Transitory computer-readable media (carrier wave and signal based) should be considered separately from non-transitory computer-readable media such as those described above. Of course, those skilled in the art will recognize many modifications may be made to this configuration without departing from the scope or spirit of the claimed subject matter.

Unless indicated otherwise, while the disclosed system is used for many different purposes (such as data collection, data analysis, data display, research, etc.), in the interest of simplicity and consistency, the overall disclosed system will be referred to hereinafter as “the system.”

In one example, the present disclosure includes a system, other class of device, and/or method to help a medical provider make clinical decisions based on a combination of molecular and clinical data, which may include comparing the molecular and clinical data of a patient to an aggregated data set of molecular and/or clinical data from multiple patients, a knowledge database (KDB) of clinico-genomic data, and/or a database of clinical trial information. Additionally, the present disclosure may be used to capture, ingest, cleanse, structure, and combine robust clinical data, detailed molecular data, and clinical trial information to determine the significance of correlations, to generate reports for physicians, recommend or discourage specific treatments for a patient (including clinical trial participation), bolster clinical research efforts, expand indications of use for treatments currently in market and clinical trials, and/or expedite federal or regulatory body approval of treatment compounds.

In one example, the present disclosure may help academic medical centers, pharmaceutical companies and community providers improve care options and treatment outcomes for patients, especially patients who are open to participation in a clinical trial.

In some embodiments of the present disclosure, the system can create structure around clinical trial data. This can include reviewing free text (i.e., unstructured data), determining relevant information, and populating corresponding structured data field with the information. As an example, a clinical trial description may specify that only patients diagnosed with stage I breast cancer may enroll. A structured data field corresponding to “stage/grade” may then be populated with “stage I,” and a structured data field corresponding to “disease type” may then be populated with “breast” or “breast cancer.” The ability of the system to create structured clinical trial data can aid in the matching of patients to an appropriate clinical trial. In particular, a patient's structured health data can be mapped to the structured clinical trial data to determine which clinical trials may be optimal for the specific patient.

In some embodiments of the present disclosure, the system can compare individual patient data to clinical trial data, and subsequently generate a report of recommended clinical trials that the patient may be eligible for. The patient's physician may review the report and use the information to enroll the patient in a well-suited clinical trial. Accordingly, physicians and/or patients do not need to manually sort and review all clinical trials within a database. Rather, a customized list of clinical trials is efficiently generated, based on the specific needs of the patient. In addition, the specific source of the patient data can easily be traced to each trial's inclusion and exclusion criteria to highlight the rationale for identifying that trial as well-suited. This generation can significantly decrease the time for a patient to find and enroll in a clinical trial, thus improving treatment outcomes for certain diseases and conditions.

In some embodiments of the present disclosure, the system can compare an individual clinical trial data to patient data at an organization, and subsequently generate a report of patients that may be eligible for that particular clinical trial. A physician, principal investigator, or clinical research administrator may review the report and use the information to enroll patients into that specific clinical trial. Accordingly, physicians and/or patients do not need to manually sort and review all patients' data to assess eligibility for a specific trial. Rather, a customized list of patients eligible for that trial is efficiently generated, based on the specific needs of the trial. This generation can significantly decrease the time for a physician, principal investigator, clinical research administrator, or other similar stakeholder to identify patients for a specific clinical trial, in part, due to the ability to reference individual source documentation for each patient's eligibility for each inclusion and exclusion criteria of the trial. Overall, the system allows for healthcare providers to track patient-level management of pre-screening, notification, consent, and enrollment into their clinical trials. Ultimately, this generation is intended to find and enroll patients in a clinical trial, thus improving treatment outcomes for certain diseases and conditions.

In some embodiments of the present disclosure, the system can facilitate activation of a new site for clinical trial participation. This can occur, in part, based on patient location to existing sites (e.g., if a patient's physician is hundreds of miles from an existing clinical trial site, a request for activation of a closer site may occur via the system) or through rapid activation of a new site. Both techniques can help to ensure that a patient can quickly enroll in a clinical trial (e.g., a nearby clinical trial), as well as quickly begin treatment. The system can provide an interface for tracking activation progress, including the various stages and corresponding tasks. As one example, a patient may submit a tissue sample and health records to a provider, receive a diagnosis, and have an available (i.e. activated) site to participate in a recommended clinical trial, all within two weeks of initial contact with the provider.

In some embodiments of the present disclosure, the system can provide an interface for sites (e.g., clinical trial sites) to submit and/or update site information in real-time. As an example, if a site installs a new machine for treatment, site personnel can update their clinical trial site information to reflect the new machine (and associated capabilities). Accordingly, the site can become eligible for a larger number of existing clinical trials, and patients can begin enrolling at the new location. The system enables providers and other users to easily update and validate their information, ensuring that patients are accurately matched with available clinical trials.

In one example, one implementation of this system may be a form of software. An exemplary system that provides a foundation to capture the above benefits, and more, is described below.

I. System Overview

In one example of the system, which may be used to help a medical provider make clinical decisions based on a combination of molecular and clinical data, the present architecture is designed such that system processes may be compartmentalized into loosely coupled and distinct micro-services for defined subsets of system data, may generate new data products for consumption by other micro-services, including other system resources, and enables maximum system adaptability so that new data types as well as treatment and research insights can be rapidly accommodated. Accordingly, because micro-services operate independently of other system resources to perform defined processes where development constraints relate to system data consumed and data products generated, small autonomous teams of scientists and software engineers can develop new micro-services with minimal system constraints that promote expedited service development.

This system enables rapid changes to existing micro-services as well as development of new micro-services to meet any data handling and analytical needs. For instance, in a case where a new record type is to be ingested into an existing system, a new record ingestion micro-service can be rapidly developed resulting in that addition of a new record in a raw data form to a system database as well as a system alert notifying other system resources that the new record is available for consumption. Here, the intra-micro-service process is independent of all other system processes and therefore can be developed as efficiently and rapidly as possible to achieve the service specific goal. As an alternative, an existing record ingestion micro-service may be modified independent of other system processes to accommodate some aspect of the new record type. The micro-service architecture enables many service development teams to work independently to simultaneously develop many different micro-services so that many aspects of the overall system can be rapidly adapted and improved at the same time.

A messaging gateway may receive data files and messages from micro-services, glean metadata from those files and messages and route those files and messages on to other system components including databases, other micro-services, and various system applications. This enables the micro-services to poll their own messages as well as incoming transmissions (point-to-point) or bus transmissions (broadcast to all listeners on the bus) to identify messages that will start or stop the micro-services.

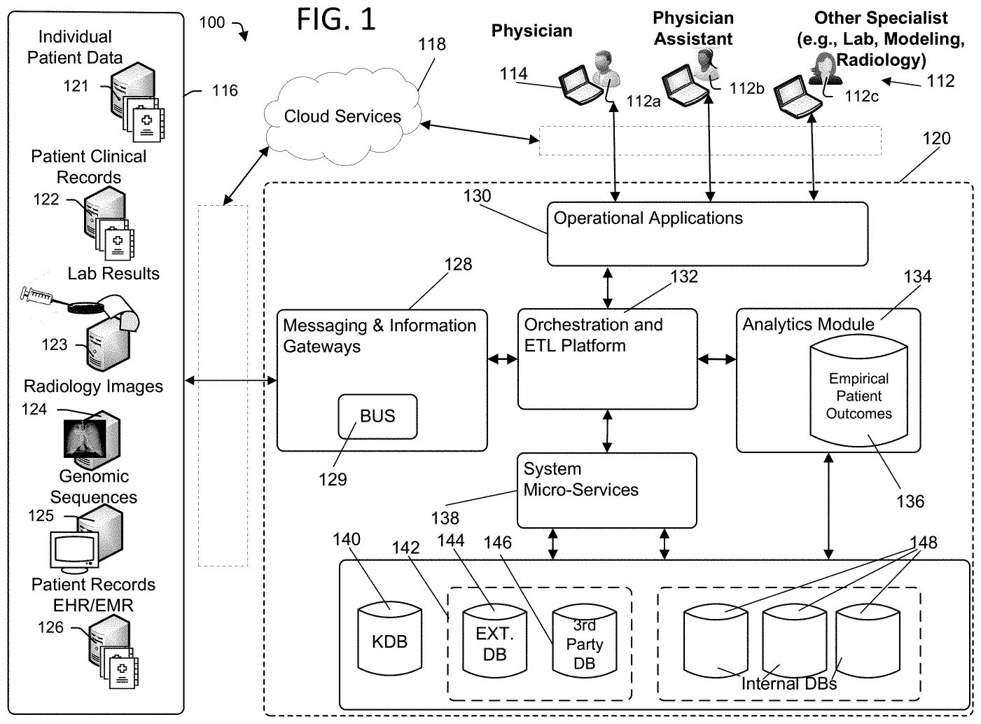

Referring now to the figures that accompany this written description and more specifically referring to FIG. 1, the present disclosure will be described in the context of an exemplary disclosed system 100 where data is shown to be received at a server 120 from many different data sources (such as database 132, clinical record 124, and micro-services (not shown)). In some aspects, the server 120 can store relevant data, such as at database 134, which is shown to include empirical patient outcomes. The server 120 can manipulate and analyze available data in many different ways via an analytics module 136. Further, the analytics module 136 can condition or “shape” the data to generate new interim data or to structure data in different structured formats for consumption by user application programs and to then drive the user application programs to provide user interfaces via any of several different types of user interface devices. While a single server 120 and a single internal database 134 are shown in FIG. 1 in the interest of simplifying this explanation, it should be appreciated that in most cases, the system 100 will include a plurality of distributed servers and databases that are linked via local and/or wide area networks and/or the Internet or some other type of communication infrastructure. An exemplary simplified communication network is labeled 118 in FIG. 1. Network connections can be any type, including hard wired, wireless, etc., and may operate pursuant to any suitable communication protocols. Furthermore, the network connections may include the communication/messaging gateway/bus that enables micro-services file and message transfer according to the above system.

The disclosed system 100 enables many different system clients to securely link to server 120 using various types of computing devices to access system application program interfaces optimized to facilitate specific activities performed by those clients. For instance, in FIG. 1 a provider 112 (such as a physician, researcher, lab technician, etc.) is shown using a display device 116 (such as a laptop computer, a tablet, a smart phone, etc.) to link to server 120. In some aspects, the display device 116 can include other types of personal computing devices, such as, virtual reality headsets, projectors, wearable devices (such as a smart watch, etc.). In some embodiments, the system 100 can include at least one processor coupled to and in communication with at least one memory.

In at least some embodiments when a physician or other health professional or provider uses system 100, a physician's user interface (such as on display device 116) is optimally designed to support typical physician activities that the system supports including activities geared toward patient treatment planning. Similarly, when a researcher (such as a radiologist) uses system 100, user interfaces optimally designed to support activities performed by those system clients are provided. In other embodiments, the physician's user interface, software, and one or more servers are implemented within one or more microservices. Additionally, each of the discussed systems and subsystems for implementing the embodiments described below may additionally be prescribed to one or more micro-systems.

System specialists (such as employees that control/maintain overall system 100) also use interface computing devices to link to server 120 to perform various processes and functions. For example, system specialists can include a data abstractor, a data sales specialist, and/or a “general” specialist (such as a “lab, modeling, radiology” specialist). Different specialists will use system 100 to perform many different functions, where each specialist requires specific skill sets needed to perform those functions. For instance, data abstractor specialists are trained to ingest clinical data from various sources (such as clinical record 124, database 132) and convert that data to normalized and system optimized structured data sets. A lab specialist is trained to acquire and process patient and/or tissue samples to generate genomic data, grow tissue, treat tissue and generate results. Other specialists are trained to assess treatment efficacy, perform data research to identify new insights of various types and/or to modify the existing system to adapt to new insights, new data types, etc. The system interfaces and tool sets available to provider specialists are optimized for specific needs and tasks performed by those specialists.

Referring again to FIG. 1, server 120 is shown to receive data from several sources. According to some aspects, clinical trial data can be provided to server 120 from database 132. Further, patient data can be provided to server 120. As shown, patient 114 has corresponding data from multiple sources (such as lab results 126 will be furnished from a laboratory or technician, imaging data 128 will be furnished from a radiologist, etc.). For simplicity, this is representatively shown in FIG. 1 as individual patient data 122. In some aspects, individual patient data 122 includes clinical record(s) 124, lab results 126, and/or imaging data 128. In some aspects, clinical record(s) 124 can include physician notes (for example, handwritten notes). The clinical record(s) 124 may include longitudinal data, which is data collected at multiple time points during the course of the patient's treatment.

The individual patient data 122 can be provided to server 120 by, for example, a data abstractor specialist (as described above). Alternatively, electronic records can be automatically transferred to server 120 from various facilities, practitioners, or third party applications, where appropriate. As shown in FIG. 1, patient data communicated to server 120 can include, but is not limited to, treatment data (such as current treatment information and resulting data), genetic data (such as RNA, DNA data), brain scans (such as PET scans, CT, MRI, etc.), and/or clinical records (such as biographical information, patient history, patient demographics, family history, comorbidity conditions, etc.).

Still referring to FIG. 1, server 120 is shown to include analytics module 136, which can analyze data from database 134 (empirical patient outcomes), and individual patient data 122. Database 34 can store empirical patient outcomes for a large number of patients suffering from the same or similar conditions or diseases as patient 114. For example, “individual patient data” for numerous patients can be associated with each respective treatment and treatment outcomes, and subsequently stored in database 134. As new patient data and/or treatment data becomes available, database 134 can be updated. As one example, provider 112 may suggest a specific treatment (e.g., a clinical trial) for patient 114, and individual patient data 122 may then be included in database 134.

The analytics module 136 can, in general, use available data to indicate a diagnosis, predict progression, predict treatment outcomes, and/or suggest or select an optimized treatment plan (such as an available clinical trial) based on the specific disease state, clinical data, and/or molecular data of each patient. In some embodiments, the analytics module 136 can include and/or execute a matching process to match a patient with a trial. An exemplary matching process is described below.

A diagnosis indication may be based on any portion of individual patient data 122 or aggregated data from multiple patients, including clinical data and molecular data. In one example, individual patient data 122 is normalized, de-identified, and stored collectively in database 134 to facilitate easy query access to the dataset in aggregate to enable a medical provider to use system 100 to compare patients' data. Clinical data may include physician notes and imaging data, and may be generated from clinical records, hospital EMR systems, researchers, patients, and community physician practices. To generate standardized data to support internal precision medicine initiatives, clinical data, including free form text, scanned documents, and/or handwritten notes, may be processed and structured into phenotypic, therapeutic, and outcomes or patient response data by methods including optical character recognition (OCR), natural language processing (NLP), and manual curation methods that may check for completeness of data, interpolate missing information, use manual and/or automated quality assurance protocols, and store data in FHIR compliant data structures using industry standard vocabularies for medical providers to access through the system 100. Molecular data may include variants or other genetic alterations, DNA sequences, RNA sequences and expression levels, miRNA sequences, epigenetic data, protein levels, metabolite levels, etc. Molecular markers specific variants or other genetic alterations, DNA sequences, RNA sequences and expression levels, miRNA sequences, epigenetic data, protein levels, metabolite levels, etc. that can indicate disruption in a patient.

As shown, outputs from analytics module 136 can be provided to display device 116 via communication network 118. Further, provider 112 can input additional data via display device 116, and the data can be transmitted to server 120. In some embodiments, provider 112 can input clinical trial information via display device 116, and the data can be transmitted to server 120. The clinical trial information can include inclusion and exclusion criteria, site information, trial status (e.g., recruiting, active, closed, etc.), among other things.

Display device 116 can provide a graphical user interface (GUI) for provider 112. The GUI can, in some aspects, be interactive and provide both comprehensive and concise data to provider 112. As one example, a GUI can include intuitive menu options, selectable features, color and/or highlighting to indicate relative importance of data. The GUI can be tailored to the type of provider, or even customized for each individual user. For example, a physician can change a default GUI layout based on individual preferences. Additionally, the GUI may be adjusted based on patient information. For example, the order of the display components and/or the components and the information contained in the components may be changed based on the patient's diagnosis, and/or the clinical trials being considered by the provider.

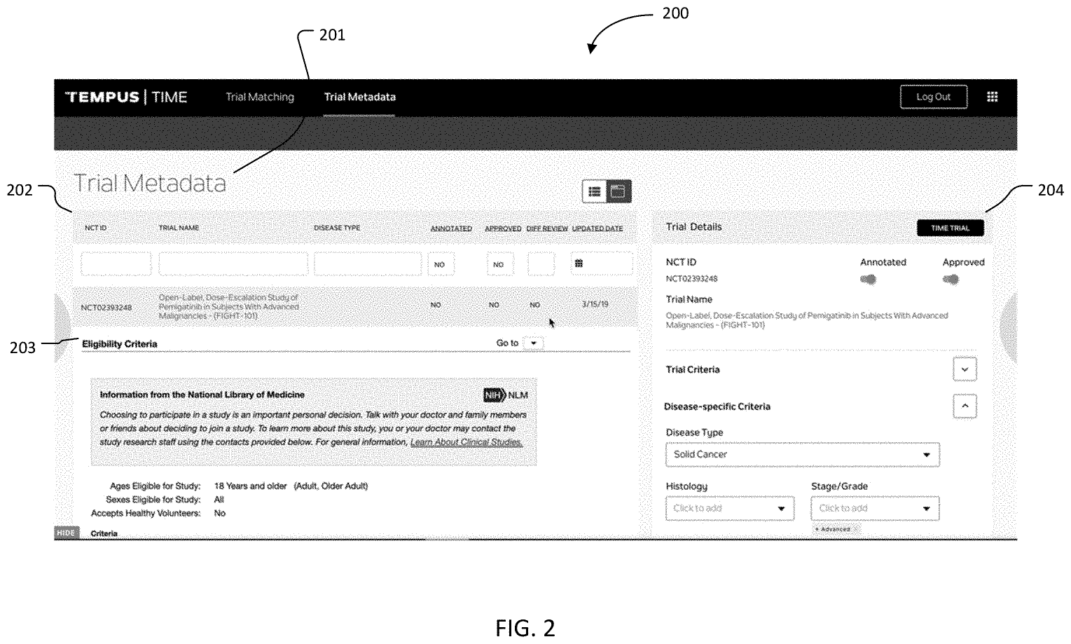

Further aspects of the disclosed system are described in detail with respect to FIGS. 2-30. In particular, an interactive GUI that can be displayed on display device 116, is shown and described.

II. Graphical User Interface

In some aspects, a graphical user interface (GUI) can be included in system 100. A GUI can aid a provider in the prevention, treatment, and planning for patients having a variety of diseases and conditions.

Advantageously, the GUI provides a single source of information for providers, while still encompassing all necessary and relevant data. This can ensure efficient and individualized treatment for patients, including matching patients to appropriate clinical trials.

In some aspects, system 100 can utilize the GUI in a plurality of modes of operation. As an example, the GUI can operate in a “trial matching” mode and a “trial construction” mode. An exemplary GUI is shown and described with respect to FIGS. 2-30.

a. Clinical Trial Data Structure

FIGS. 2-9 generally provide graphical user interfaces (GUIs) that can be implemented in system 100 to structure data (e.g., clinical trial data). In some aspects, reports that flow for clinical patients can rely on recommendations and suggestions on which clinical trials the patient is eligible for, as well as clinical and molecular insights. In order to do that effectively, unstructured clinical trial data can be structured using free-text (unstructured data) sourced from clinical trial databases and/or websites (e.g., clinicaltrials.gov). Notably, many clinical trial databases and websites contain clinical trials that are available to the public. Some clinical trials and/or clinical trial information remain private, and can be protocol-specific from various sponsors (e.g., pharma sponsors). Regardless of public or private status, structured clinical trial data can be used in a variety of ways, including to match patients to appropriate clinical trials.

FIG. 2 is shown to include a graphical user interface (GUI) 200. In some aspects, GUI 200 can include a first portion corresponding to trial metadata 201. As shown, trial metadata 201 can further include trial data 202, a trial description 203, and trial details 204.

Trial metadata 201 can be used to view, update, and sort data corresponding to clinical trials. As shown, for example, the trial data 202 can be summarized via a displayed table on GUI 200. The trial data 202 can include separate table entries for each clinical trial. As an example, each clinical trial may be listed with the corresponding national clinical trial (NCT ID), the trial name, the disease type relating to the clinical trial, an annotation status, an approved status, a review status, and/or the date of last update.

In some aspects, a user can select an individual clinical trial. GUI 200 may subsequently display the corresponding trial description 203. The trial description 203 may be sourced directly from a clinical trials database or website. Accordingly, the text included within the trial description 203 may be unstructured data. As will be described, a user may view the trial description 203 and enter relevant trial criteria into the trial details 204. In other situations, optical character recognition (OCR) and/or natural language processing (NLP) may be used to map the trial description 203 to the appropriate data fields within the trial details 204.

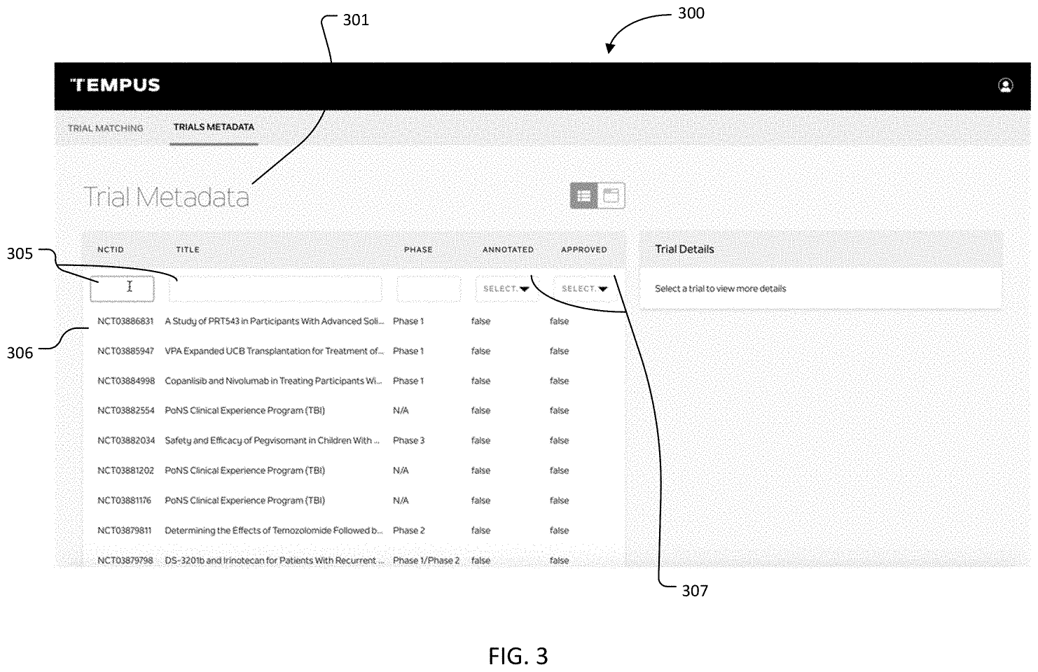

FIG. 3 is shown to include a graphical user interface (GUI) 300. In some aspects, GUI 300 can include a first portion corresponding to trial metadata 301. As shown, trial metadata 301 can further include text fields 305, a table 306, and/or selection menus 307.

Trial metadata 301 can be used to view, update, and sort data corresponding to clinical trials. As shown, for example, various text fields 305 can be used to filter a large number of clinical trials, based on user-entered text. In some aspects, a user can filter the listing of clinical trials by entering full or partial text-strings corresponding to the NCT ID, clinical trial title, recruitment status, cancer type, molecular inclusion/exclusion, gene, an annotation status, an approved status, trial program type, and/or phase of the clinical trial. As an example, a user may enter “1” into the “phase” text field 305, and GUI 300 may subsequently display only clinical trials that are described as “phase 1” or similar.

In some aspects, a user can provide a selection via selection menus 307. Similar to the filtering that can occur based on user-entered text, a user can filter the listing of clinical trials via selection menus 307. In some aspects, selection menus 307 can be provided for the “annotated” and/or “approved” criteria, as shown by FIG. 3. Selection menus 307 may be dropdown menus, for example, and selection options may include “true” and “false,” or “yes” and “no.” In other aspects, selection options and menus can vary (e.g., “phase” criteria may be configured to have a selection menu). Notably, a user may enter text and/or selections into multiple fields at once, to further filter the listed clinical trials.

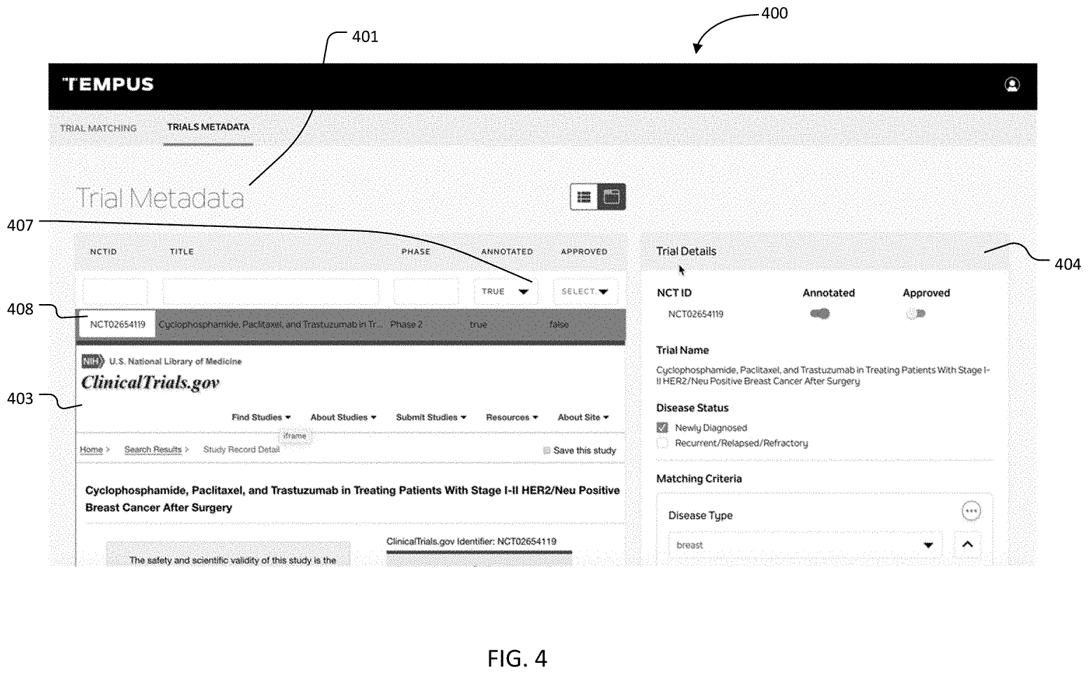

FIG. 4 is shown to include a graphical user interface (GUI) 400. In some aspects, GUI 400 can include a first portion corresponding to trial metadata 401. As shown, trial metadata 401 can further include selection menus 407, a trial header 408, a trial description 403, and/or trial details 404.

As an example, the “annotated” selection menu 407 has been set to “true.” Accordingly, clinical trials that match the selected annotation criteria are displayed via the GUI 400. An example clinical trial is shown in FIG. 4. The trial header 408 is shown to include the NCT ID “NCT02654119,” the title “Cyclophosphamide, Paclitaxel . . . ,” the phase (“phase 2”), a “true” indicator of annotation, and a “false” indicator of approval. The trial description 403 can be sourced from clinicaltrials.gov, for example. Accordingly, the clinicaltrials.gov page that is associated with the selected clinical trial can be displayed.

In some aspects, the trial details 404 can include a set of fields that a user may optionally add information to. In some situations, the data within the trial description 403 may include substantially unstructured data (free-text). Accordingly, the sourced raw data may be relatively useless in the context of clinical informatics. The free-text therefore inhibits the ability to compare data in a programmatic or dynamic way.

As shown by FIG. 4, the trial details 404 can include the “annotated” and “approved” statuses, the trial name, the trial NCT ID, the disease status, and a portion corresponding to “matching criteria.” A data abstractor (or other user) can utilize GUI 400, in the context of system 100, to create structure around the clinical trial by evaluating source text (unstructured data), and filling in relevant information within the trial details 404.

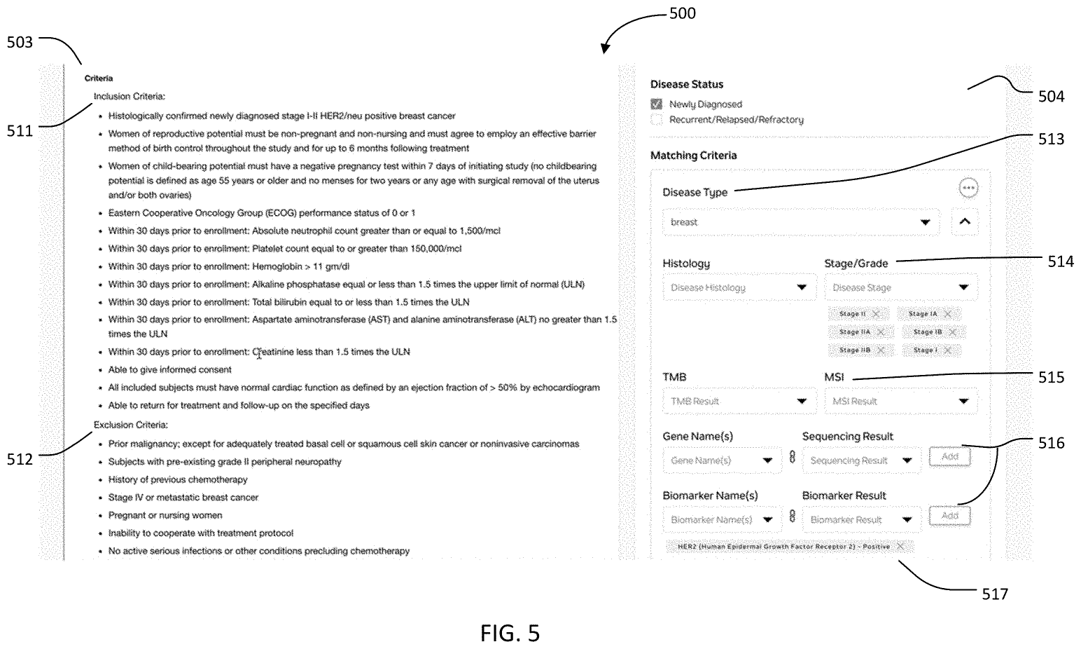

FIG. 5 is shown to include a graphical user interface (GUI) 500. In some aspects, GUI 500 can include trial description 503, and trial details 504. As shown, the trial description 503 can include inclusion criteria 511 and exclusion criteria 512. Further, as shown, the trial details 504 can include disease criteria 513, stage/grade criteria 514, genetic criteria 515, add button(s) 516, and/or biomarker criteria 517.

As an example, the first element shown within the inclusion criteria 511 is “histologically confirmed newly diagnosed stage I-II HER2/neu positive breast cancer.” Accordingly, within the trial details 504, “newly diagnosed” may be selected (e.g., checked), the disease criteria 513 may be selected (or otherwise input) as “breast,” and the stage/grade criteria may include “stage II, stage I, stage IIA, IIB, IA, IB.” Using GUI 500, the free-text within the inclusion criteria 511 may be mapped/associated with existing structured data fields. In some aspects, the existing structured data fields (e.g., disease criteria 513, etc.) can align with the structured data fields that may be used to capture patient data. In some situations, it may be desirable to have very granular information. Therefore, the various matching criteria fields may be fairly granular. The specificity of the matching criteria fields can enable accurate comparisons between patient data and clinical trial eligibility data, for example.

Notably, there may be several methods for creating structured data fields, such as the fields shown in FIG. 5. In some aspects, for example, system 100 may include structured data fields previously defined within an electronic medical record (EMR) or electronic data warehouse (EDW) maintained by a healthcare provider. Alternatively, system 100 may include existing structured data fields from a database maintained by a clinical laboratory, such as a laboratory that provides DNA and/or RNA sequencing; analysis of imaging features; organoid laboratory services; or other services. In some aspects, system 100 may utilize existing structured data fields from electronic data warehouses, hospitals, and health information exchanges, among other sources. In other aspects, the structured data fields may be a set of data fields appropriate for the structuring of clinical trial inclusionexclusion criteria.

Still referring to FIG. 5, an example biomarker “HER2 (Human Epidermal Growth Factor Receptor 2)—Positive” is shown to be selected within the biomarker criteria 517. This biomarker selection corresponds to the first element listed within the inclusion criteria 511. Accordingly, the system 100 can be enabled to qualify the specific biomarker, and the result that corresponds to it.

In some aspects, a data abstractor (or other users of the system 100) can select a biomarker name (for example) from the biomarker name dropdown menu. Subsequently, the data abstractor can select a biomarker result from the biomarker result dropdown menu. Once the data abstractor has selected all desired elements, they may select “add.” In some aspects, selecting “add” can create a new filter, which may be displayed via GUI 500. Displayed filters can indicate to users which active filters meet the inclusion or exclusion criteria of the clinical trial.

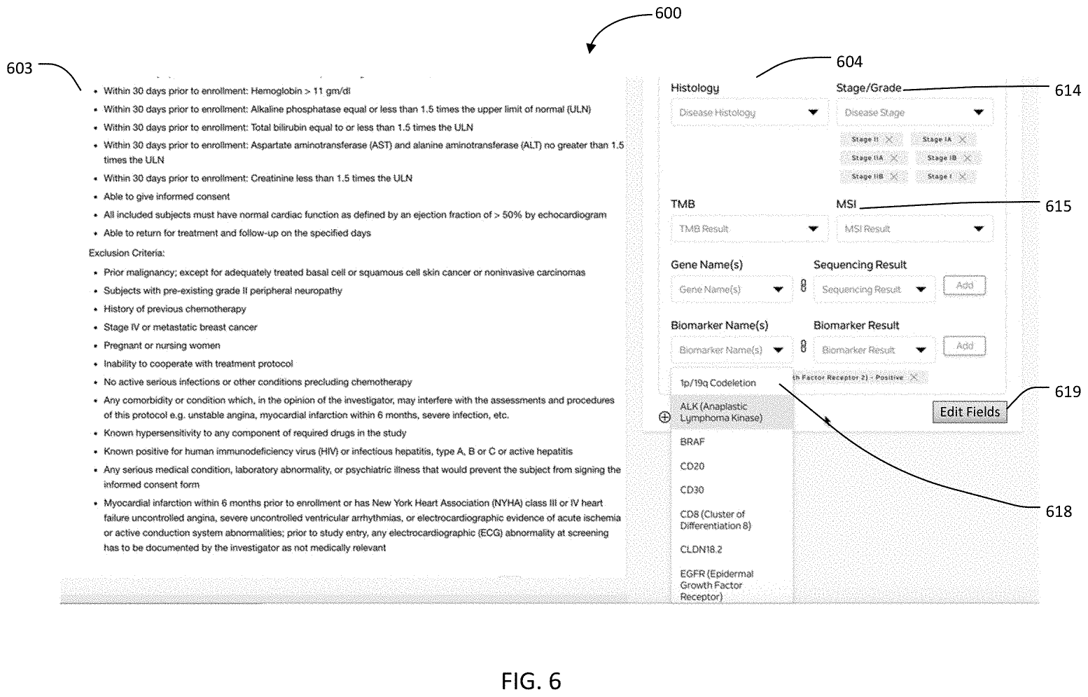

FIG. 6 is shown to include a graphical user interface (GUI) 600. In some aspects, GUI 600 can include trial description 603, trial details 604, stage/grade criteria 614, genetic criteria 615, selection menu 618, and button 619.

As shown, selection menu 618 can be a dropdown menu. As an example, selection menu 618 can include several known biomarker names (e.g., “ALK,” “BRAF,” etc.). In some aspects, the trial description 603 can be abstracted and assigned to a category. Exemplary categories can include an “inclusion” category and an “exclusion” category. In some aspects, the inclusion category can be denoted by a specific color, and the exclusion category can be denoted with a second, specific color. Accordingly, a data abstractor can now identify if an element is present within the trial description 603, in addition to specifying whether or not it should be present within the patient data of potential clinical trial participants. As one example, a clinical trial may specify that patients who received prior treatments may be disqualified from participating. As another example, exclusion criteria 512 may include certain vaccines, such as cancer vaccines (e.g., an HPV vaccine).

Still referring to FIG. 6, button 619 can be configured to edit the fields available (and displayed) to the user. In some aspects, the fields shown to be included within the trial details 604 can be added or removed by a data abstractor (or other user), as desired. Selection of the button 619 can provide a menu of available fields and/or fields currently in-use on GUI 600. Adding and/or removing fields enables a data abstractor to locate the correct fields that can be used for mapping the inclusion criteria from the trial description 603, while preventing clutter of GUI 600. As an example, an RNA field is shown in FIG. 6, but the trial description 603 does not have criteria relating to RNA. Accordingly, a data abstractor may select button 619 and proceed to remove the RNA field from the trial details 604. Further, associated fields (e.g., RNA sequencing results) may be automatically removed in response to a field being removed. Conversely, when a field is added, associated fields may be automatically added and displayed.

As mentioned above, a natural language processing (NLP) tool can be implemented within the system 100. NLP can analyze the trial description 603, and provide a preliminary determination of which data fields may be relevant to the specific clinical trial. Accordingly, certain data fields may be automatically removed or added within the trial details 604. As an example, if the NLP tool does not detect a performance score status of ECOG in the trial description (shown in FIG. 6), a user may not be prompted to fill in an ECOG status or score. System 100 may include a machine learning tool that can review the trial description 603, as well as the criteria listed within the description, and make a determination about what structured data fields could be appropriate to include in the trial details 604. The user can still have control over adding and/or removing fields, but the machine learning tool and/or NLP tool can provide an informed starting point for data abstraction. Accordingly, users may be able to efficiently and accurately complete the trial details 604.

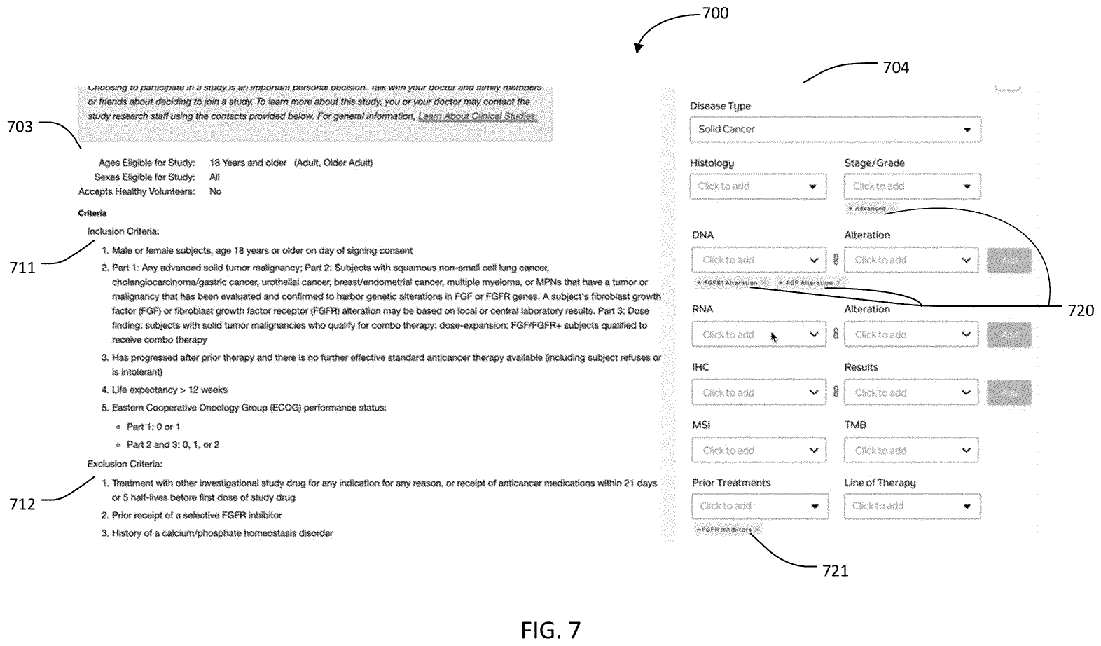

FIG. 7 is shown to include a graphical user interface (GUI) 700. In some aspects, GUI 700 can include trial description 703, trial details 704, inclusion criteria 711, exclusion criteria 712, inclusion attributes 720, and exclusion attributes 721.

As shown in FIG. 7, inclusion attributes 720 may be indicated by a first color (e.g., green), and exclusion attributes 721 may be indicated by a second color (e.g., red). In some aspects, other methods of distinction may be implemented. As an example, the inclusion attributes 720 may be indicated via a first text identifier, and the exclusion attributes 721 may be indicated via a second text identifier.

In some aspects, the natural language processing (NLP) tool can be configured to provide predictive text, based on the trial description 703. As an example, the system 100 can pre-populate “FGFR1 Alteration” and “FGFR Inhibitors” into the respective data fields (DNA, prior treatments), as shown in FIG. 7. In some aspects, a data abstractor may verify the pre-populated data, but the system 100 can provide an informed suggestion.

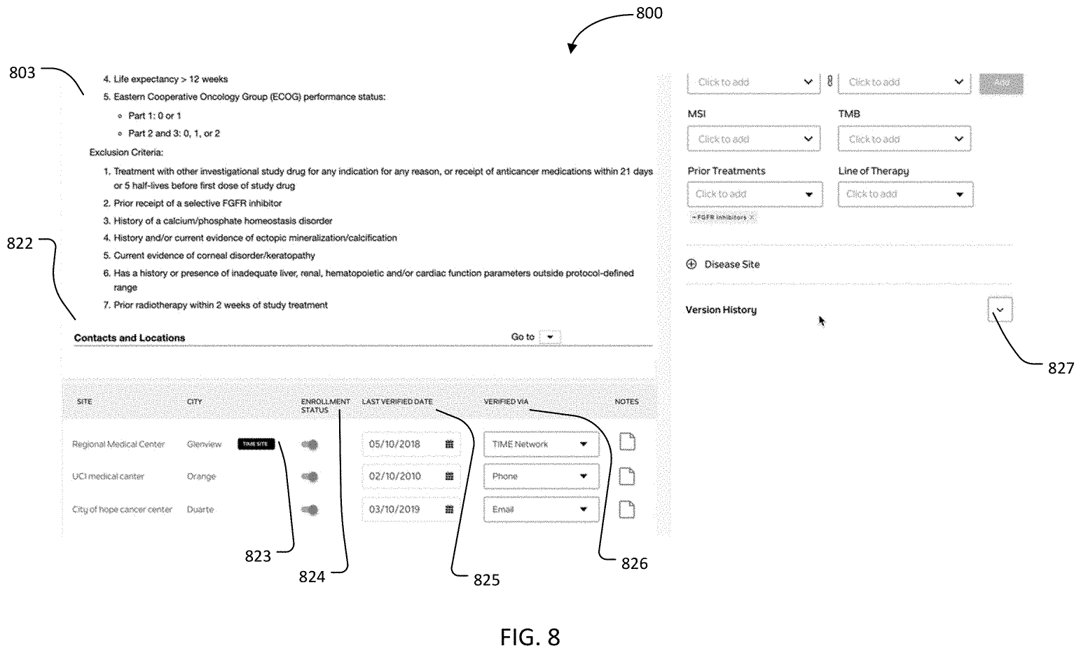

FIG. 8 is shown to include a graphical user interface (GUI) 800. In some aspects, GUI 800 can include trial description 803, trial location(s) 822, identifier 823, enrollment status 824, verification date 825, verification method 826, and version history button 827.

In some aspects, GUI 800 can display a version history when version history button 827 is selected. The version history view may be limited, based on the user's role within the system 100. In some aspects, the version history can include a table with information corresponding to what change occurred, the user ID (or name) corresponding to the change, and a time stamp when the change occurred. The version history can capture changes made by a system user via the GUI 800, as well as changes that occurred within the source data. As an example, if a clinical trial provider added a new trial site, the GUI 800 may subsequently indicate the site availability. The version history can display the addition of the site as a time stamped change. Advantageously, the system 100 can provide a version history of every clinical trial that is being annotated. This aspect can be beneficial in situations where clinical trial data must be abstracted and entered into structured data fields, as well as separately verified and approved by another user.

For each clinical trial, there is at least one, and potentially thousands of sites where the trial can be conducted/administered. As an example, FIG. 8 shows a trial that has three sites. Notably, in other clinical trials, there may be a very long list of sites. In some aspects, the list of sites can be categorized based on different health systems, different sites, satellite offices, etc. Each table listing can include the site name, the location (e.g., city), an enrollment status 824 (e.g., “enrolling” or “closed”), the last verification date 825, the verification method 826 (e.g., phone, email, etc.), and/or corresponding notes. The verification information can ensure that any recommended clinical trials have up-to-date and accurate data. As shown, an identifier 823 can be added to specific sites. In some aspects, the identifier 823 can be displayed by site listings where the site was activated via the system 100.

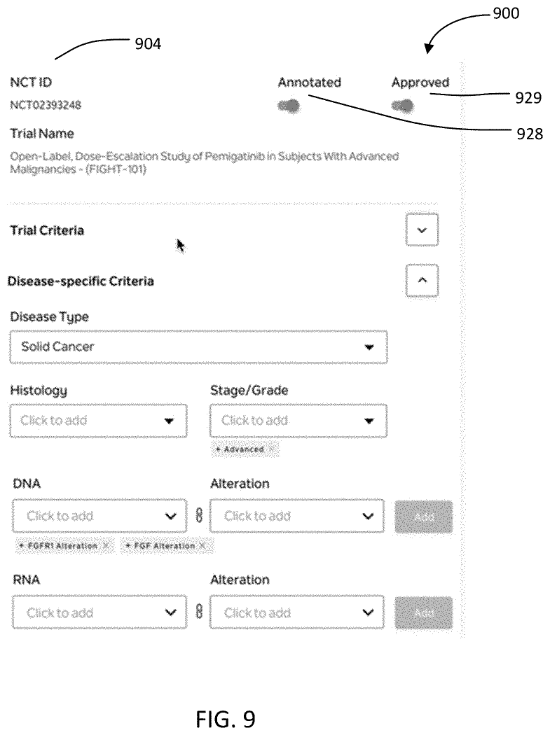

FIG. 9 is shown to include a graphical user interface (GUI) 900. In some aspects, GUI 900 can include trial details 904, an annotation indicator 928, and an approval indicator 929.

In some aspects, a data abstractor (or other user) can select the annotation indicator 928 to provide an indication that changes have been made to the trial details 904. This can, in some aspects, generate an alert for another user (e.g., a supervisor, manager, etc.) that an annotation requires approval. The second user may verify the changes made to the trial details 904, and can subsequently select the approval indicator 929. In some aspects, the changes may not be reflected within the system 100 until the approval indicator 929 has been selected. This verification step can ensure that changes and updates accurately reflect the clinical trial data.

In some aspects, system 100 can integrate with clinical trial management systems that are configured and available “on premise.” Generally, on premise systems are administered via cloud services. Further, on premise systems are predominantly focused on demographic information about a patient, for example, their medical record number (MRN), name, birth date, etc. All other data often requires a separate system, or alternatively, system users do not have visibility into all of the clinical and molecular traits that are needed to enroll or disqualify a patient from a trial. In some aspects, existing on premise systems can be used to determine the enrollment and recruiting status of a site, as well as if a patient with a certain MRN has successfully enrolled at the site. The other information (as described above) is not present within on premise systems, and instead may be spread between clinical documents and notes, which contain unstructured data.

The GUIs described above (e.g., GUIs 200-900) can generally be used by a system administrator to associate existing clinical trials with structured data fields.

b. Clinical Trial Matching

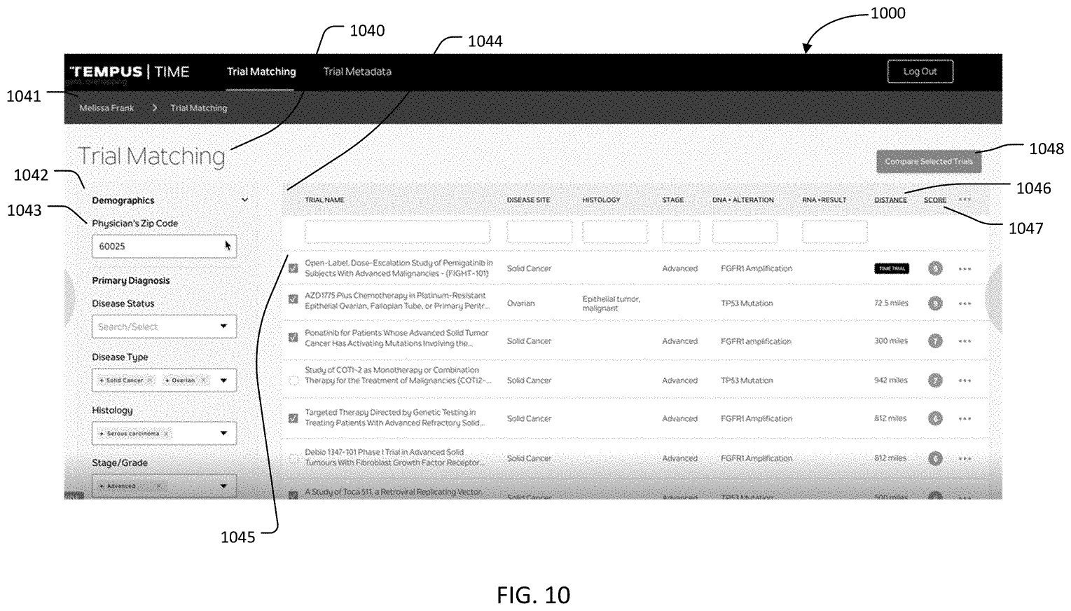

FIGS. 10-15 generally provide graphical user interfaces (GUIs) that can be implemented in system 100 to appropriately match patients with available clinical trials. As described above, reports that flow for clinical patients can rely on recommendations and suggestions on which clinical trials the patient is eligible for, as well as clinical and molecular insights.

FIG. 10 is shown to include a graphical user interface (GUI) 1000. In some aspects, GUI 1000 can include a portion corresponding to trial matching 1040, a patient identifier 1041, patient demographics 1042, a physician location 1043, a table 1044, trial selectors 1045, a distance 1046, a score 1047, and/or a comparison button 1048.

In some aspects, GUI 1000 can be configured for a physician or other provider for identifying trials that are the most appropriate for their patients. As an example, GUI 1000 shows information for a patient, Melissa Frank. The patient identifier 1041 can include the patient's name, an ID number, etc. The trial matching 1040 can include the patent demographics 1042, such as disease status, disease type, etc. The combination of attributes shown for the patient can be provided using similar methods as the above-described “trial metadata” data abstraction. Accordingly, a user can view and/or enter all of the relevant information corresponding to the patients and diseases. This can enable system 100 to correctly match clinical trial elements with patient data (e.g., histology, stage/grade, disease type, etc.).

Notably, in some aspects, the trial matching 1040 can include the physician location 1043, which may be indicated by the zip code of the physician's office (e.g., the office that the patient is typically seen at). The physician location 1043 can be used to find clinical trial sites within a certain distance of the physician, for example. In some aspects, the zip code may be prepopulated in the physician location field 1043. The zip code may be determined by the physician name and/or the name of the patient.

As shown, the table 1044 can include a list of clinical trials that match the patient's specific data (as indicated on the left side of GUI 1000). System 100 can be configured to analyze and compare patient data to the clinical trial data. Further, system 100 can provide the table 1044 based on clinical trials that substantially align with patient data. Each clinical trial within the table 1044 can include a trial selector 1045, a trial name, a disease site, histology data, disease stage, DNA data, RNA data, distance 1046 (e.g., from the physician's zip code), and/or a “score” 1047. In some aspects, the table 1044 can be sorted based on user-specified criteria (e.g., by distance, by score, etc.).

Still referring to FIG. 10, a user can select (e.g., via trial selectors 1045) one or more clinical trials to see more information, and/or compare the clinical trials to one another. Once one or more clinical trials have been selected, a user can select the comparison button 1048.

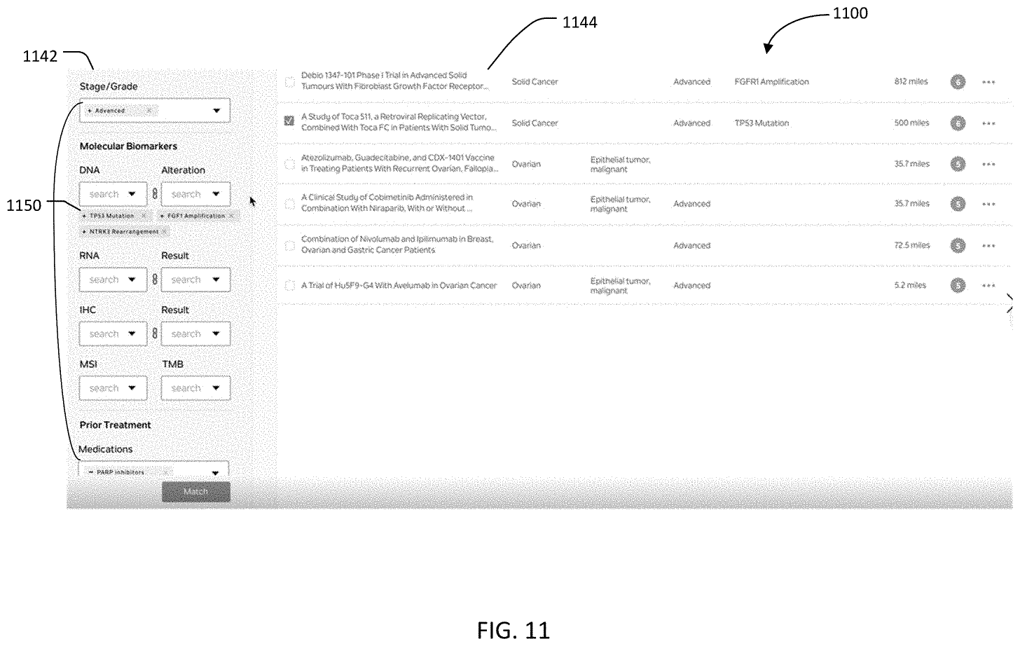

FIG. 11 is shown to include a graphical user interface (GUI) 1100. In some aspects, GUI 1100 can include patient demographics 1142, a table 1144, and/or attributes 1150.

As shown by FIGS. 10-11, the clinical data corresponding to the patient Melissa Frank is already prepopulated via the attributes 1150. By “disease type” for example, a user can see that Melissa has solid cancer (ovarian), histology is a serous carcinoma, the cancer is in an advanced stage, and Melissa has certain mutations, amplifications, and rearrangements. In some aspects, the clinical data can come from a structured clinical data source (e.g., an EMR, a clinical lab record, an electronic data warehouse, a health information exchange, etc.). System 100 can prepopulate the attributes 1150 based on the structured clinical data.

Once the patient data has been provided, a user can select “match.” The match function can determine and provide a score (e.g., the highest score listed first) of clinical trial matches. The score can be based on the disease site, the histology, the stage, molecular information, as well as the distance. In some aspects, other matching criteria may be implemented. In some aspects, there may be different methods to match a patient's health information to trial inclusion and exclusion criteria. As an example, FIGS. 10-11 include a match score. In some aspects, a binary “yes” or “no” may be used as a match indicator. As mentioned above, each of the listed trials can be selected for comparison and/or inclusion within a patient report.

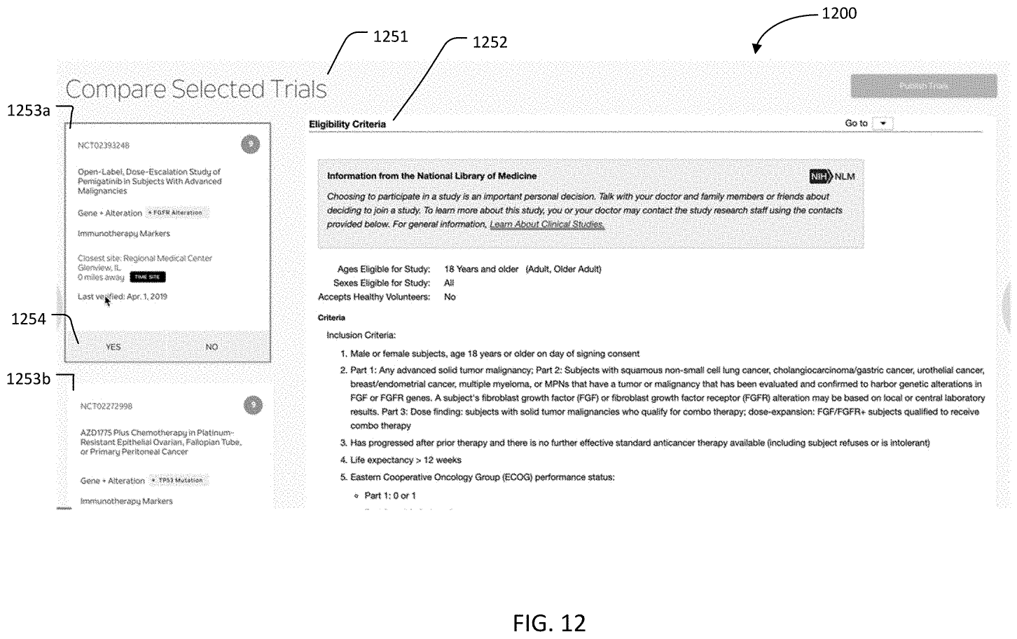

FIG. 12 is shown to include a graphical user interface (GUI) 1200. In some aspects, GUI 1200 can include a trial comparison 1251, eligibility criteria 1252, selected trials 1253a, 1253b, and/or yes/no selector 1254.

As shown, the trial comparison 1251 can include a list of selected trials 1253a, 1253b. Each selected trial 1253 can include summary details specific to the clinical trial. As an example, a user may be presented with the NCT ID, the score, a summary of all the relevant biomarkers, the site(s), and the last verification time stamp. Further, a user may view comprehensive clinical trial information (e.g., the eligibility criteria 1252) by selecting an individual trial from the list of selected trials 1253a, 1253b. In some aspects, a user can toggle “yes” or “no” via the yes/no selector 1254. Selecting “no” may remove the clinical trial from the selected trial list, according to some aspects.

In some aspects, GUI 1200 can display inclusion criteria matched directly to the patient clinical data elements (e.g., via a table). A color indicator (e.g., red or green) may be provided to reflect whether or not the patient meets the particular criteria. The color indicator can advantageously provide a secondary verification, such that a user can quickly discern if a data entry error occurred.

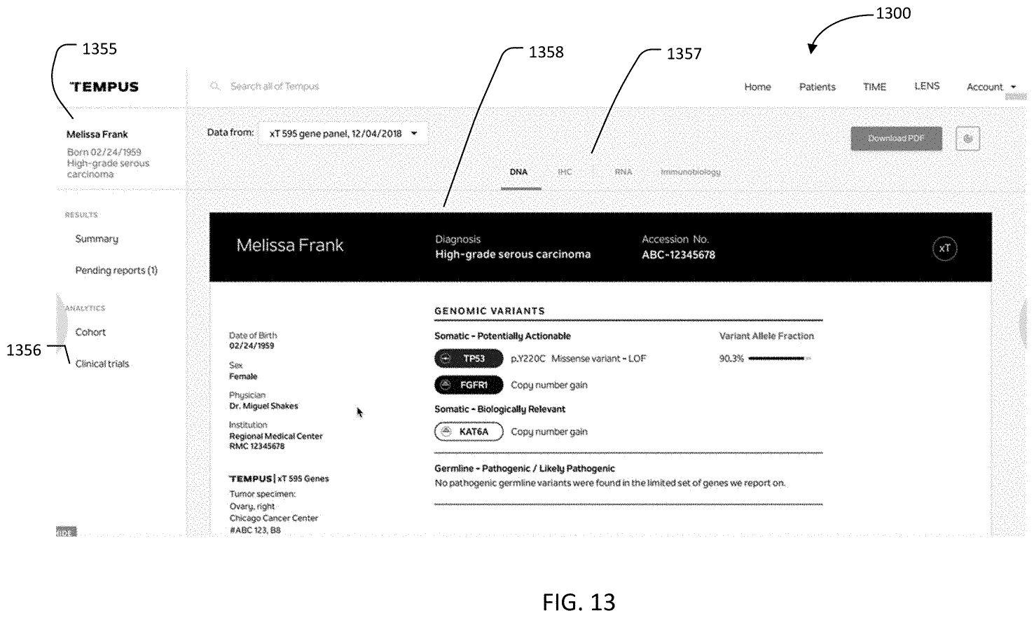

FIG. 13 is shown to include a graphical user interface (GUI) 1300. In some aspects, GUI 1300 can include a patient summary 1355, a clinical trials tab 1356, a patient data menu 1357, and/or patient data 1358.

In some aspects, GUI 1300 can display a match report for the patient. The system 100 can generate the match report based on the suggested and finalized clinical trials. As shown, the patient summary 1355 can include information such as patient name, date of birth, and/or primary diagnosis. Additionally, the patient data menu 1357 can be configured to toggle between various patient information (e.g., DNA, IHC, RNA, and Immunology). As an example, “DNA” is shown to be selected from the patient data menu 1357. Accordingly, the patient data 1358 that is shown corresponds to the patient's DNA information. In some aspects, the generated report can include molecular markers, information about specimens and tissues, tests that have been run, as well as all the clinical trials that the patient matched.

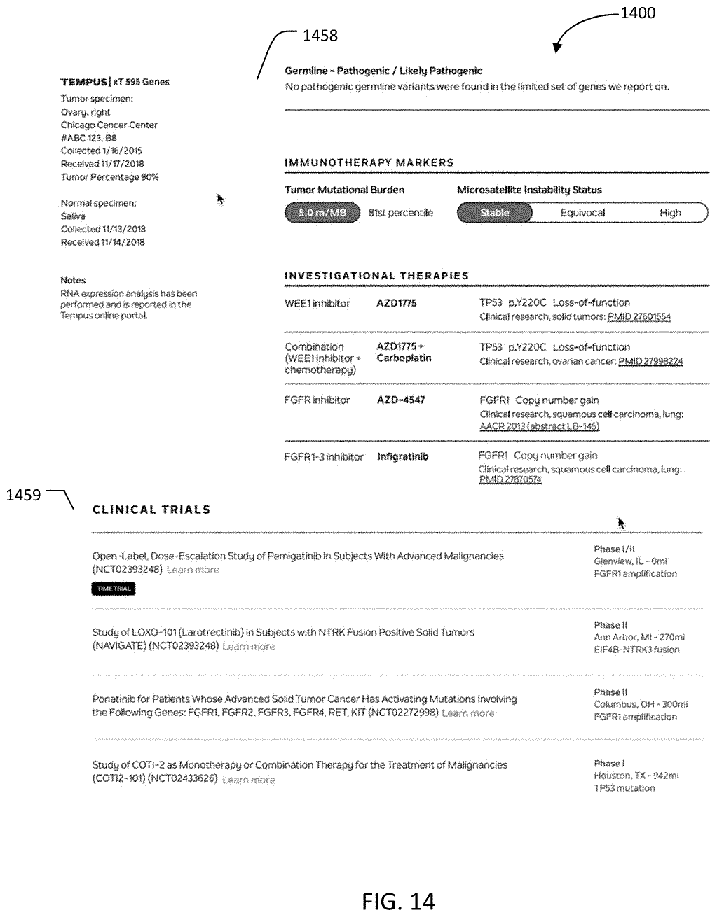

FIG. 14 is shown to include a graphical user interface (GUI) 1400. In some aspects, GUI 1400 can include patient data 1458 and/or a table 1459. The table 1459 can include all trials that have been selected for this patient, as an example. Further, each of the clinical trials can be selected to view more information.

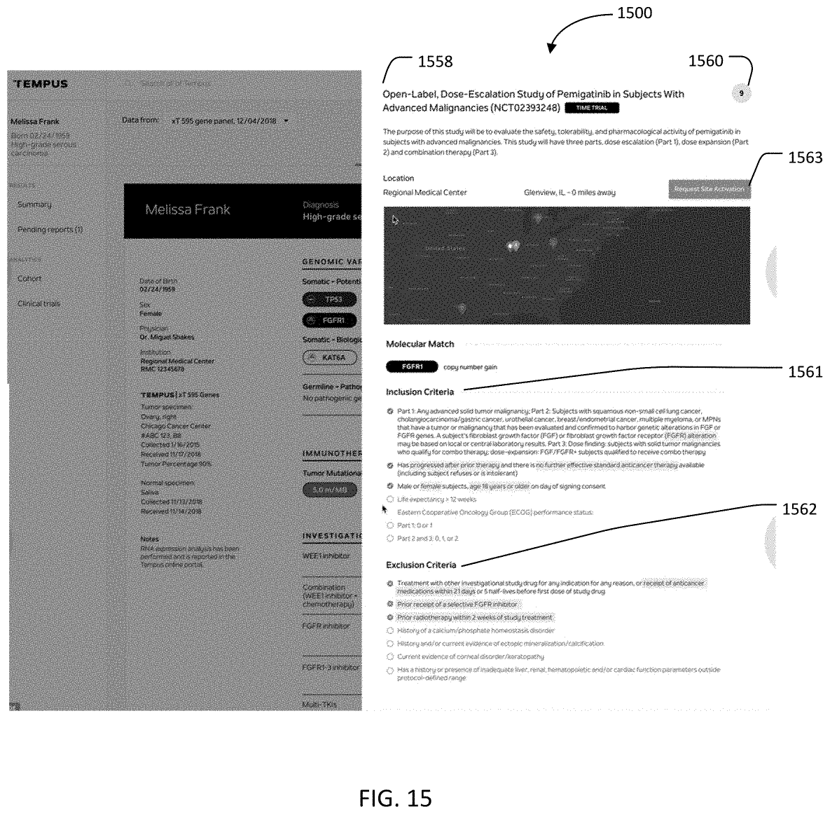

FIG. 15 is shown to include a graphical user interface (GUI) 1500. In some aspects, GUI 1500 can include a clinical trial description 1558, a score 1560, inclusion criteria 1561, exclusion criteria 1562, and/or a site activation button 1563.

As shown, additional details (e.g., the clinical trial description 1558) relating to the clinical trial may be displayed upon selection. The additional details can include the score 1560 that corresponds to the specific patient being matched. In some aspects, information about the inclusion and exclusion criteria can be displayed as matched to the patient. As an example, the GUI 1500 can color code and highlight (e.g., with green and red) the inclusion criteria 1561 and exclusion criteria 1562, based on data that has been successfully matched to the criteria that the trial has defined.

In some aspects, a user can select the site activation button 1563 to begin a “rapid site activation.” A rapid site activation can include matching eligible clinical patients with sponsored protocols (e.g., private clinical trials), and activating a new site for the primary purpose of conducting the specific sponsored protocol. In some aspects, a site (e.g., a physician's organization), may request activation of a new site for a clinical trial. As an example, FIG. 15 shows the physician “Dr. Miguel Shakes,” as well as the institution associated with the physician, Regional Medical Center. Accordingly, Regional Medical Center may request a site activation for this particular clinical trial.

c. Clinical Trial Site Activation

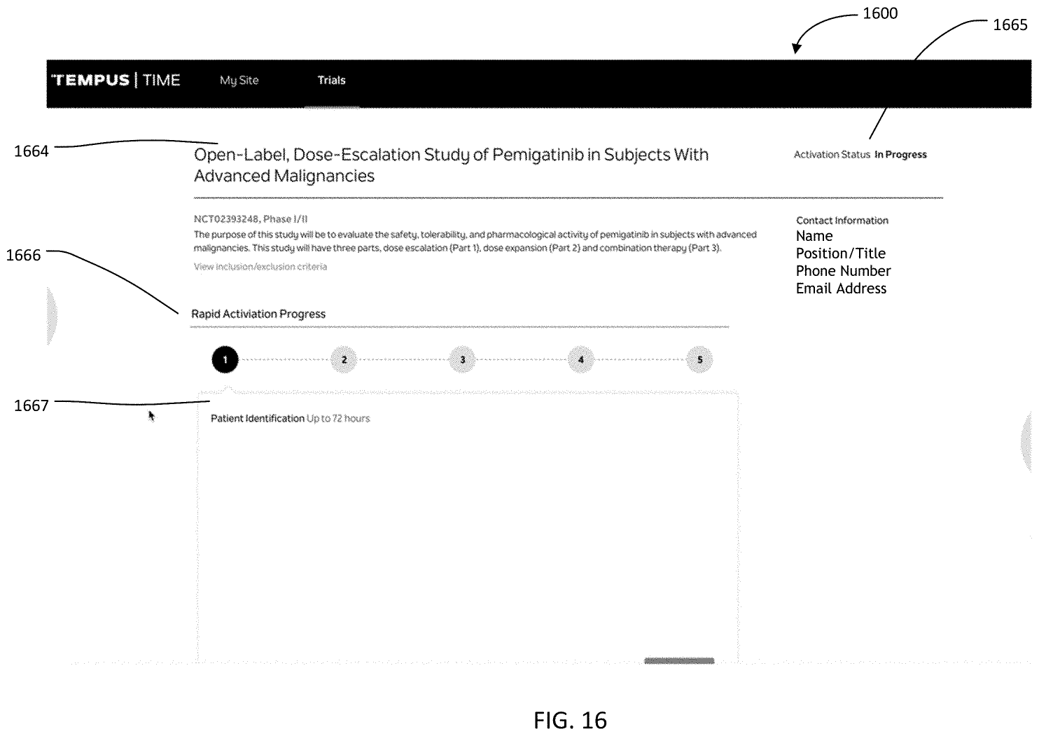

FIGS. 16-20 generally provide graphical user interfaces (GUIs) that can be implemented in system 100 to activate new clinical trial sites. In some aspects, site activation can occur in response to a patient being matched to a clinical trial. Alternatively, site activation can occur as-needed during enrollment of a clinical trial. Rapid activation of a new site can enable fast patient enrollment and subsequent treatment. Further, rapid activation can aid researchers in recruiting optimally matched patients.

FIG. 16 is shown to include a graphical user interface (GUI) 1600. In some aspects, GUI 1600 can include a clinical trial summary 1664, an activation status indicator 1665, a progress indicator 1666, and/or progress information 1667.

In some aspects, the process of rapid site activation can occur in two weeks or less. As an example, a patient may provide their information and/or samples to a physician, and within two weeks be enrolled in a clinical trial at a newly activated site. As shown in FIG. 16, the clinical trial summary 1664 can be displayed via the GUI 1600. Additionally, contact information for the site and/or clinical trial can be displayed. The progress indicator 1666 can track the various “stages” of site activation. In some aspects, the rapid site activation process can be divided into five main stages.

As shown, the progress information 1667 can include a list of elements that should be completed within the respective stages. In some aspects, the list of elements can be updated in real-time, via GUI 1600. Elements may appear as incomplete or complete, and may be updated by the various system users. As shown, a first stage of the rapid site activation process can be “patient identification,” and the stage can take up to 72 hours, as an example. In some aspects, the activation status indicator 1665 can display if the activation status is in progress or complete.

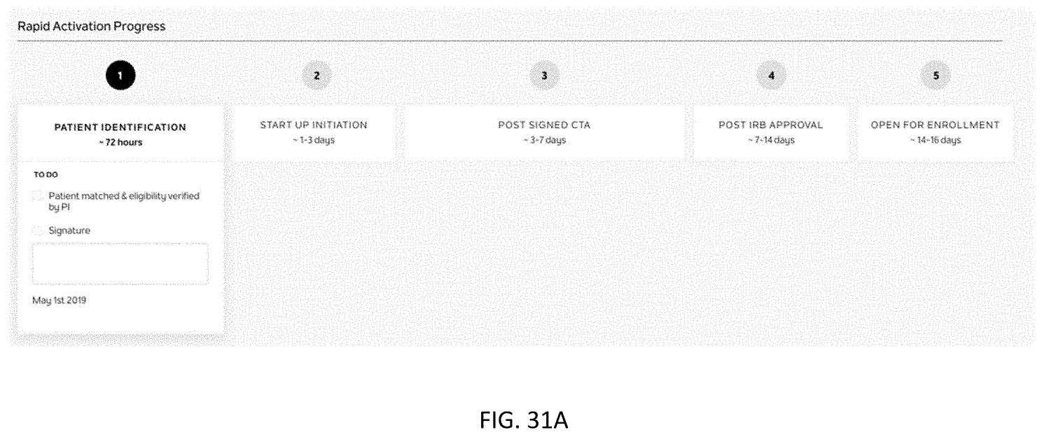

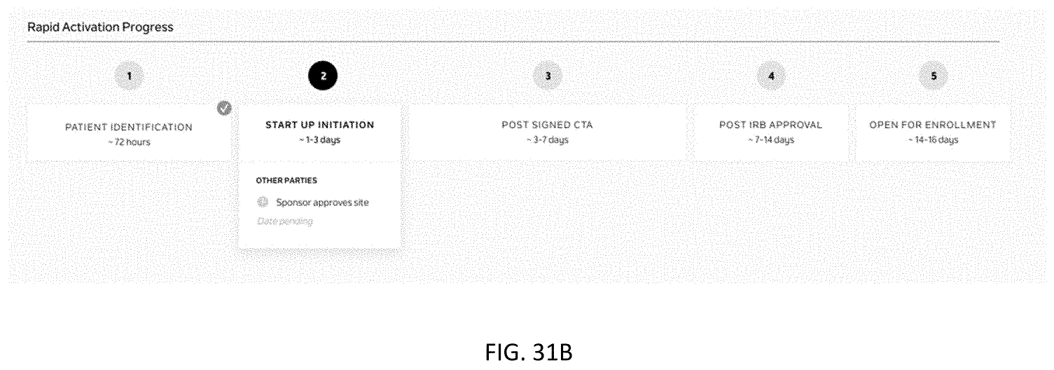

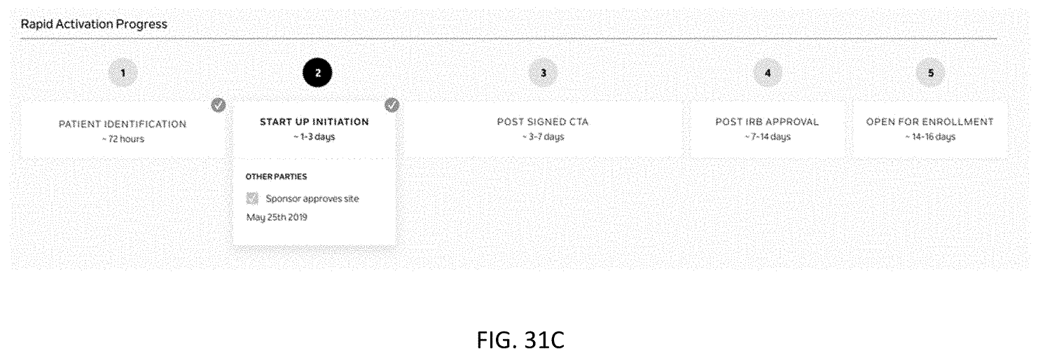

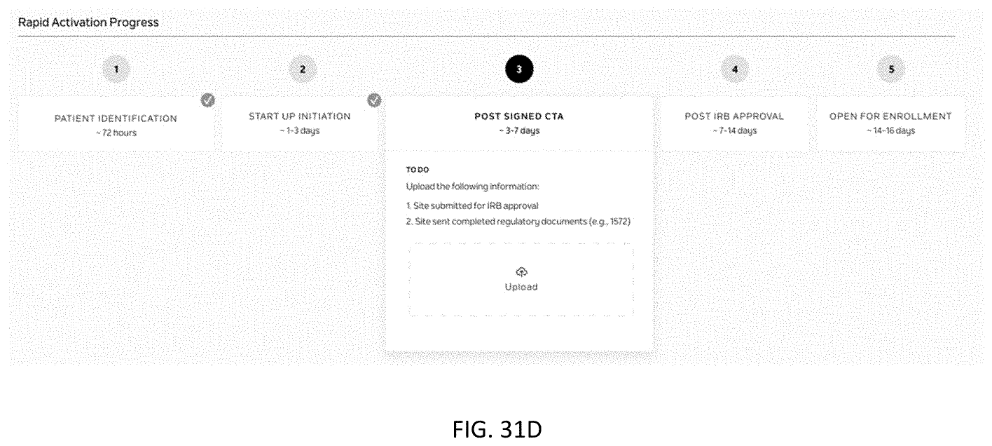

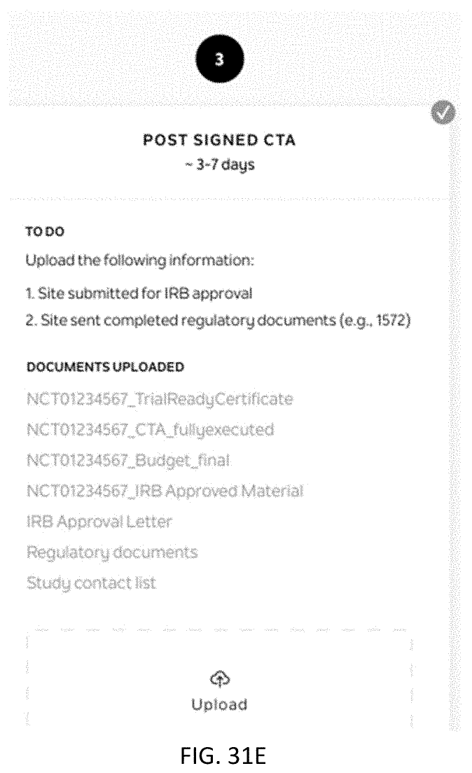

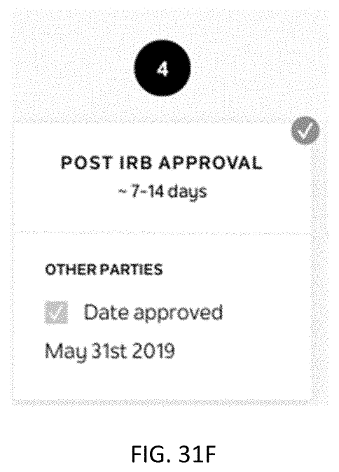

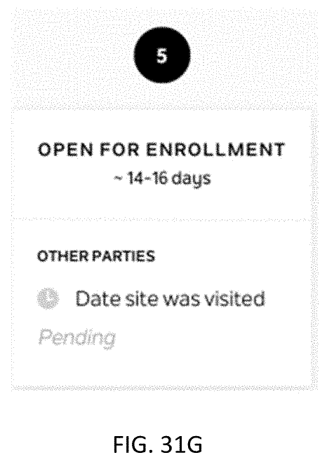

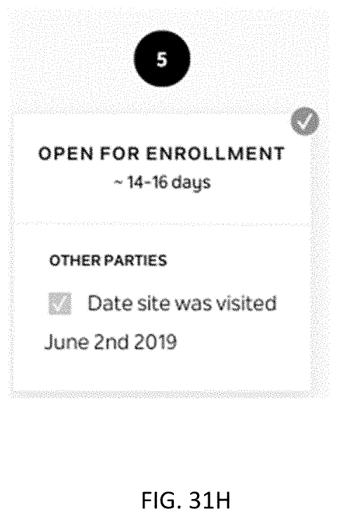

FIG. 31A is shown to include a GUI that lists items that are required in order to complete a patient identification stage. The physician (indicated here as the PI) confirms that the patient matches the inclusion/exclusion criteria and can electronically sign to confirm the same. The information is transmitted electronically for review by the sponsor. In some aspects, the information used to validate the inclusion/exclusion criteria confirmation (either structured or unstructured) may be sent to the study sponsor or designee for review and/or confirmation.

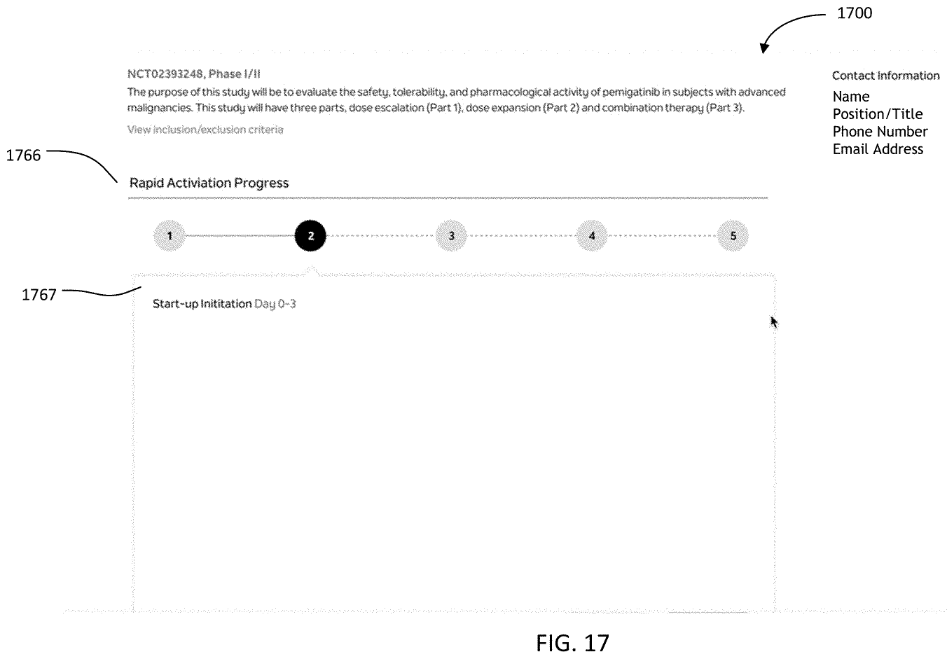

FIG. 17 is shown to include a graphical user interface (GUI) 1700. In some aspects, GUI 1700 can include a progress indicator 1766, and progress information 1767.

As shown, the progress information 1767 can include a list of elements that should be completed within the respective stages. In some aspects, the list of elements can be updated in real-time, via GUI 1700. Elements may appear as incomplete or complete, and may be updated by the various system users. As shown, a second stage of the rapid site activation process can be “start-up initiation,” and the stage can last from day 0 to day 3, as an example.

FIG. 31B is shown to include a GUI that may be included within the progress information 1767 in various aspects. FIG. 31B may include a notice to the site that the sponsor's approval is pending. The GUI within the progress information 1767 may be updated dynamically once the sponsor has provided approval. FIG. 31C is shown to include a GUI that includes a notice to the site that the sponsor has approved the site and the date of approval.

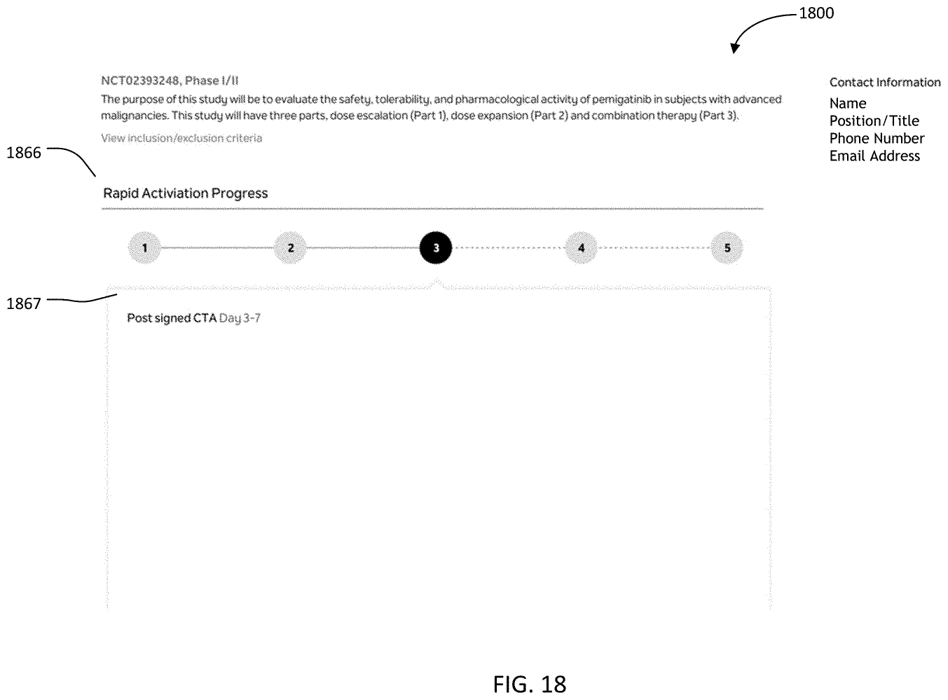

FIG. 18 is shown to include a graphical user interface (GUI) 1800. In some aspects, GUI 1800 can include a progress indicator 1866, and progress information 1867.

As shown, the progress information 1867 can include a list of elements that should be completed within the respective stages. In some aspects, the list of elements can be updated in real-time, via GUI 1800. Elements may appear as incomplete or complete, and may be updated by the various system users. As shown, a third stage of the rapid site activation process can be “post-signed CTA” (post-signed Clinical Trial Agreement), and the stage can last from day 3 to day 7, as an example.

FIG. 31D is shown to include a GUI that is included within the progress information 1867 in various aspects. FIG. 31D includes a “to do” listing of information that needs to be uploaded for transmission to the sponsor. Examples include IRB approval submission information and regulatory documents, such as the Form 1572 required by the FDA. The GUI may include an interactive upload element that permits a file to be dragged and dropped into the element. Such action causes the file to be transferred through a computer network and uploaded to a remote server for further review. The GUI within the progress information 1867 may be updated dynamically once the sponsor has provided approval. FIG. 31E shows the names of files that have been uploaded, such as a trial ready certificate, the fully executed clinical trial agreement, the study budget, the IRB approved patient materials, the IRB approval letter, regulatory documents, and the study contact list.

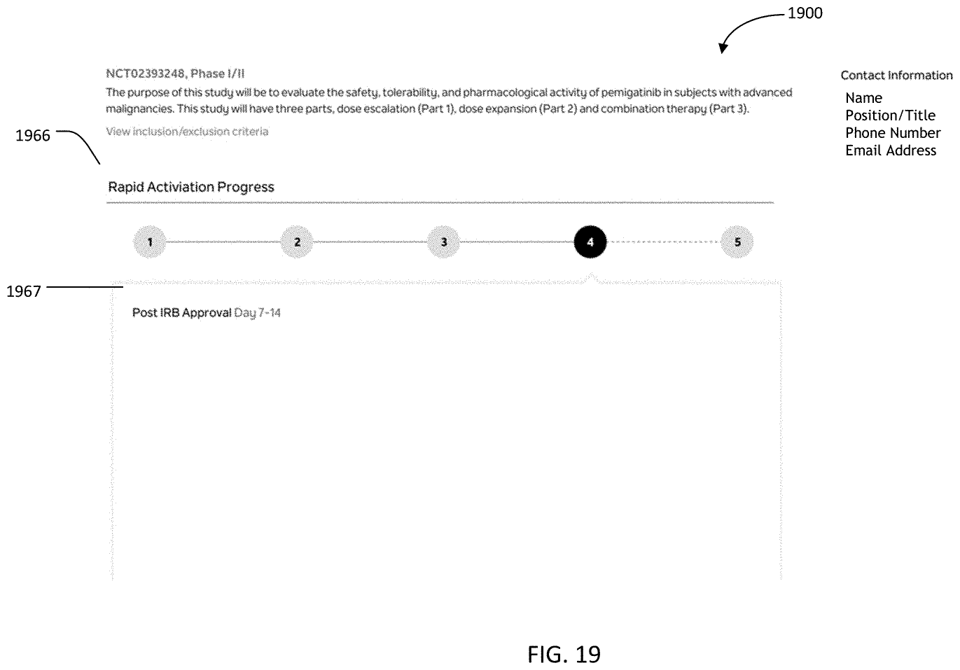

FIG. 19 is shown to include a graphical user interface (GUI) 1900. In some aspects, GUI 1900 can include a progress indicator 1966, and progress information 1967.

As shown, the progress information 1967 can include a list of elements that should be completed within the respective stages. In some aspects, the list of elements can be updated in real-time, via GUI 1900. Elements may appear as incomplete or complete, and may be updated by the various system users. As shown, a fourth stage of the rapid site activation process can be “post-IRB approval” (post-Institutional Review Board approval), and the stage can last from day 7 to day 14, as an example.

FIG. 31F is shown to include a GUI that is included within the progress information 1967 in various aspects. The GUI includes a confirmation that the IRB approved the study and the date of approval.

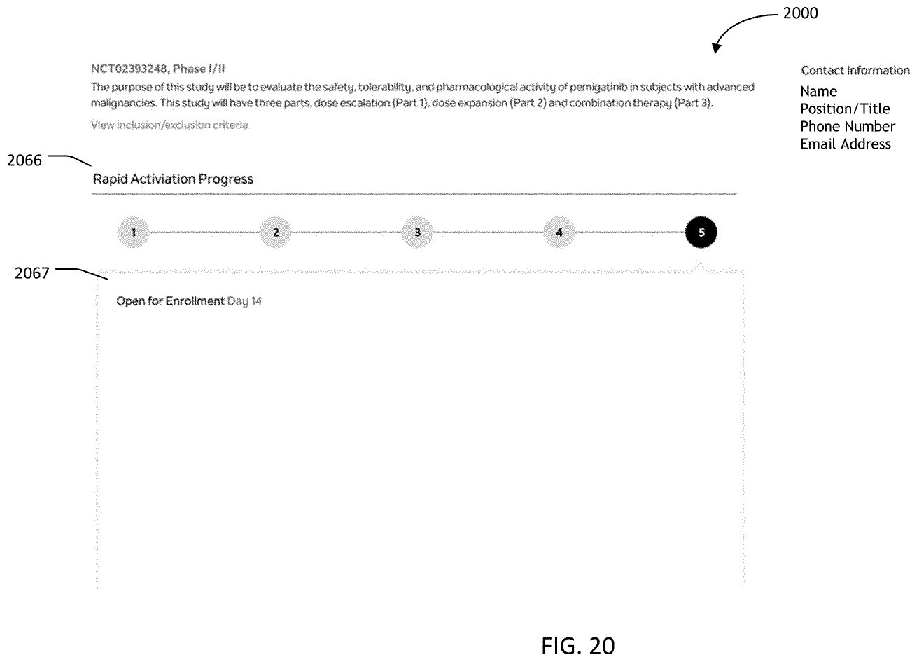

FIG. 20 is shown to include a graphical user interface (GUI) 2000. In some aspects, GUI 2000 can include a progress indicator 2066, and progress information 2067.

As shown, the progress information 2067 can include a list of elements that should be completed within the respective stages. In some aspects, the list of elements can be updated in real-time, via GUI 2000. Elements may appear as incomplete or complete, and may be updated by the various system users. As shown, a fifth stage of the rapid site activation process can be “open for enrollment,” which can be the last stage, occurring on day 14.

FIG. 31G is shown to include a GUI that is included within the progress information 2067 in various aspects. The GUI includes a notice that the site visit is pending. The GUI in the progress information 2067 may be dynamically updated. For example, once the site visit has occurred, the GUI in the progress information 2067 may reflect that the site was visited and the visit date, as shown in the GUI included in FIG. 31H.

In some aspects, once the rapid site activation process is complete, the site can open for enrollment. Accordingly, the patient can be eligible to begin the clinical trial at the newly activated site.

d. Clinical Trial Site Information

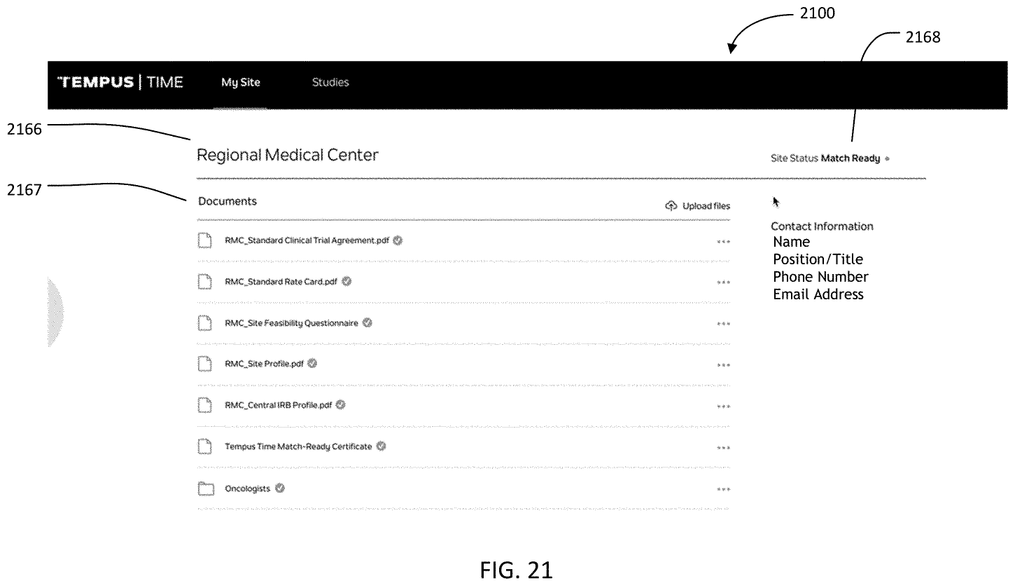

FIGS. 21-30 generally provide graphical user interfaces (GUIs) that can be implemented in system 100 to track and/or update site capabilities in relation to clinical trials. In some aspects, initial site information can be input via GUIs 2100 -3000. Further, as site equipment and/or capabilities change, users on-site can update the site information in real-time. This ensures that clinical trials can be matched to patients and corresponding sites, without relying on outdated and potentially incorrect information. In some aspects, on-site users (e.g., site administrators) can log in and have access to the site information that is stored within system 100. The site information may apply to multiple clinical trials, and system 100 accordingly provides interfaces that enable centralized data entry.

FIG. 21 is shown to include a graphical user interface (GUI) 2100. In some aspects, GUI 2100 can include a site name 2166, site documents 2167, and/or a site status 2168. As shown, the site status 2168 can indicate that the site is ready for patient matching.

In some aspects, GUI 2100 can display a list of site documents 2167. Sites may run multiple clinical trials, and system 100 provides a central access point for site information. As shown, for example, Regional Medical Center has multiple categories of associated documents.

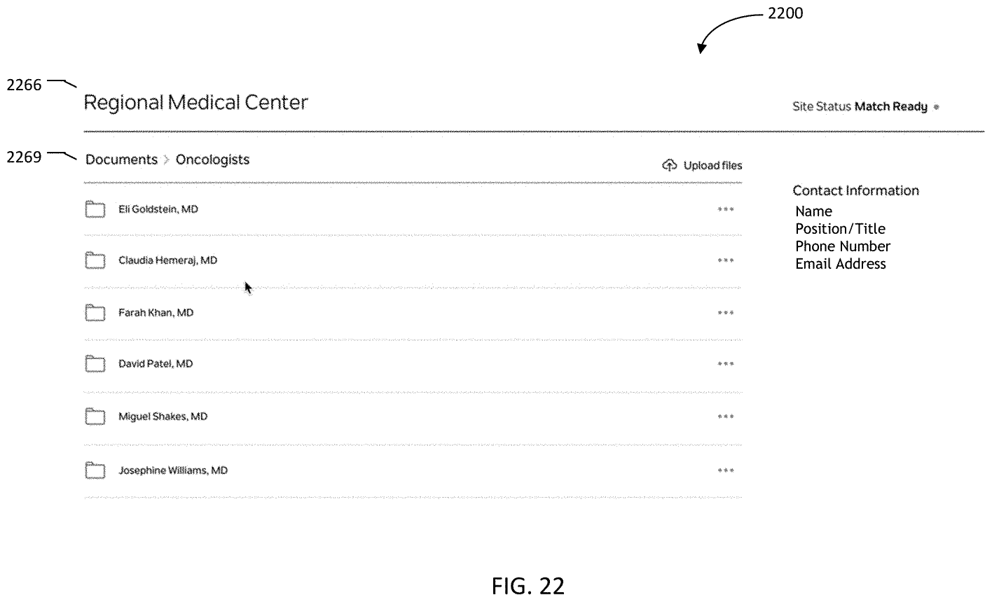

FIG. 22 is shown to include a graphical user interface (GUI) 2200. In some aspects, GUI 2200 can include a site name 2266, and a documents list 2269.

In some aspects, GUI 2200 can display a documents list 2269 corresponding to each oncologist related to Regional Medical Center, as an example. A user can select a specific oncologist to see additional information.

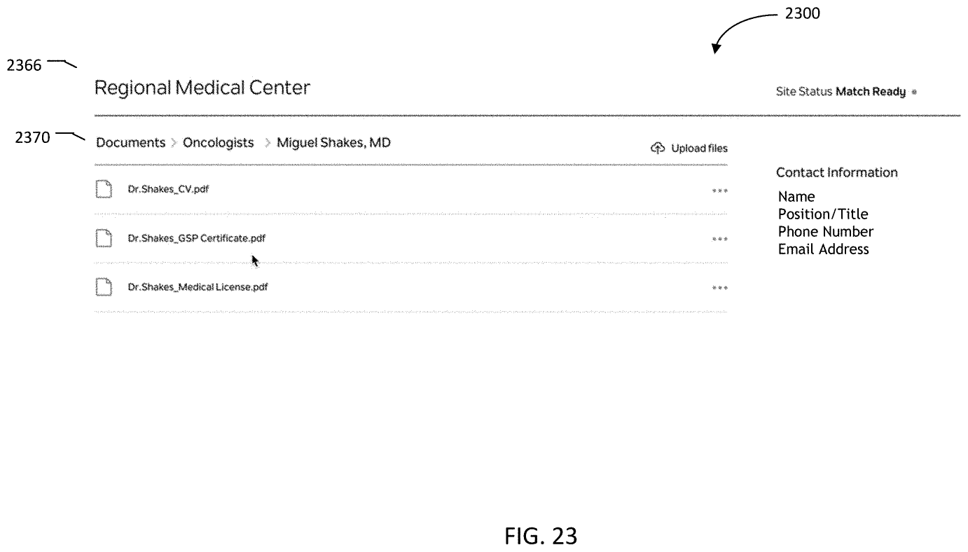

FIG. 23 is shown to include a graphical user interface (GUI) 2300. GUI 2300 is shown to include a site name 2366, and physician documents 2370.

In some aspects, GUI 2300 can display a list of physician documents 2370. As shown, for example, a user can view the documents related to a specific physician. In some aspects, the documents can include the physician's CV, resume, certificates, and/or medical license.

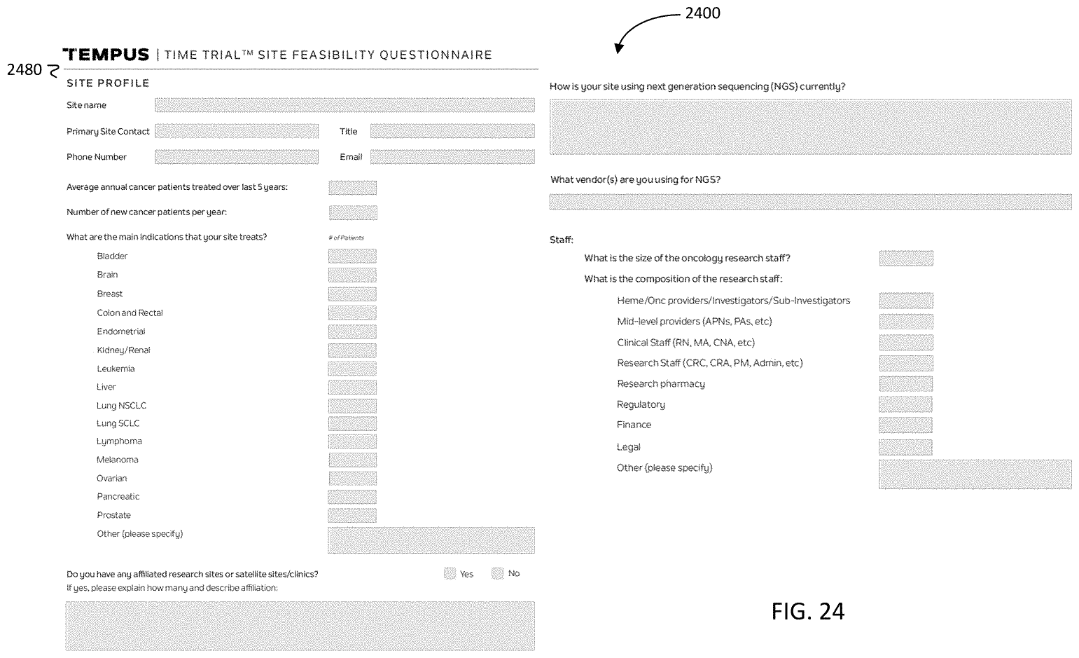

As described above, users can view and/or update site capabilities using system 100. As site capabilities change, users can update the site information in real-time, for example. FIGS. 24-30 provide example GUIs corresponding to obtaining site information. Notably, FIGS. 24-30 relate specifically to sites conducting oncology clinical trials, but the general concepts described herein can be applied to any disease or condition.

FIG. 24 is shown to include a graphical user interface (GUI) 2400. In some aspects, GUI 2400 can include a site profile 2480. In some aspects, GUI 2400 can be configured for user inputs, which can subsequently update site information within system 100.

In some aspects, the site profile 2480 can include fields corresponding to the site name, the primary site contact, and/or staffing information. Further, the site profile 2480 can include fields corresponding to specific disease areas (e.g., number of cancer patients treated, types of cancers treated, etc.).

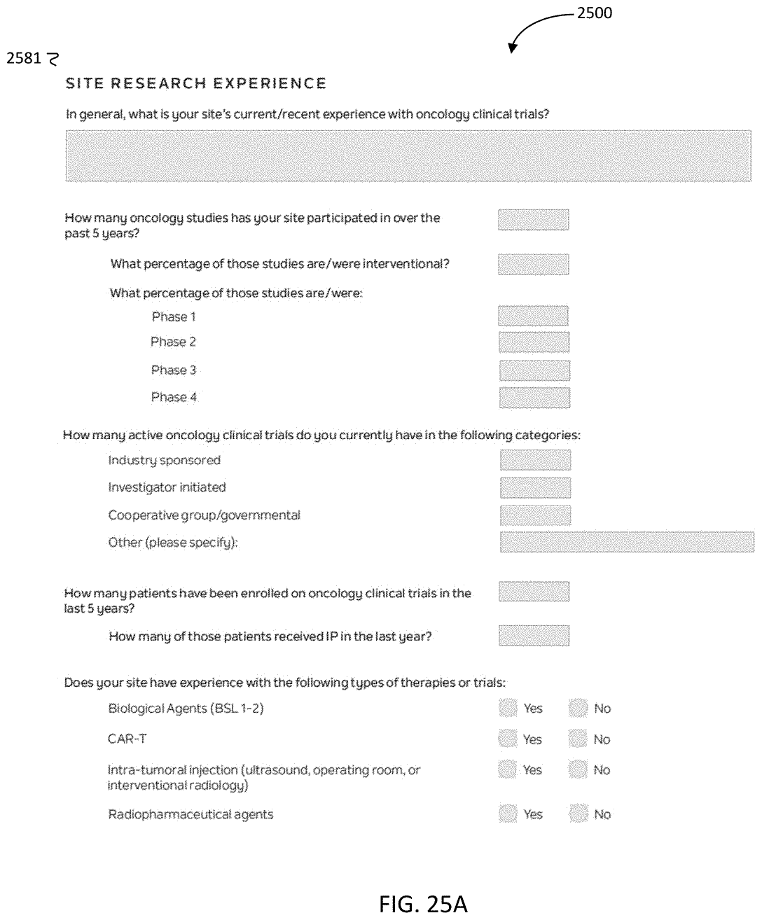

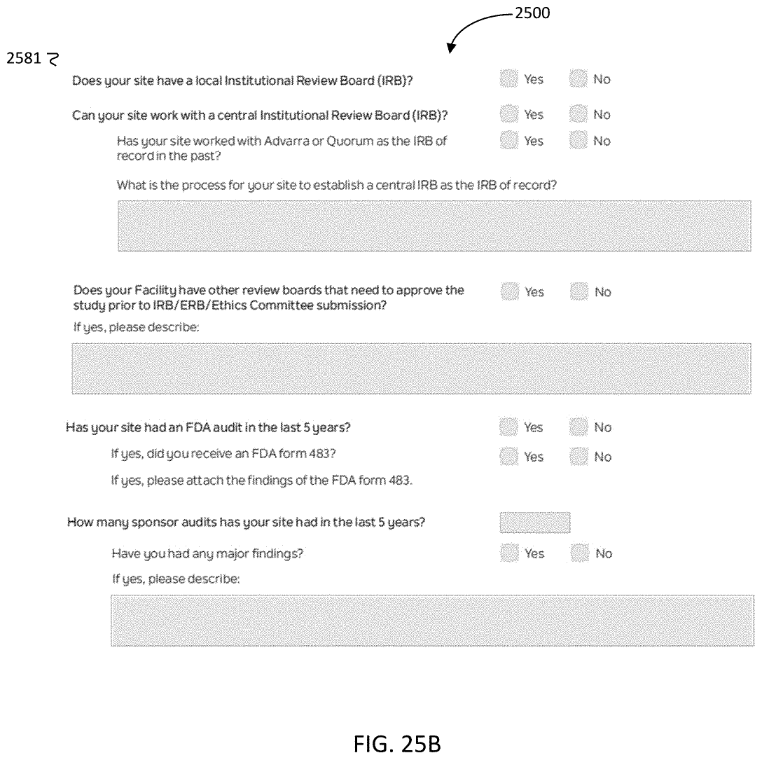

FIGS. 25A-25 B are shown to include a graphical user interface (GUI) 2500. In some aspects, GUI 2500 can include site research experience 2581. In some aspects, GUI 2500 can be configured for user inputs, which can subsequently update site information within system 100. Further, the site research experience 2581 can include experiences with an IRB and/or ethics committee, and regulatory agencies (e.g., the FDA).

In some aspects, the site research experience 2581 can include recent experience with clinical trials, number of studies participated in, and/or sponsor types, for example.

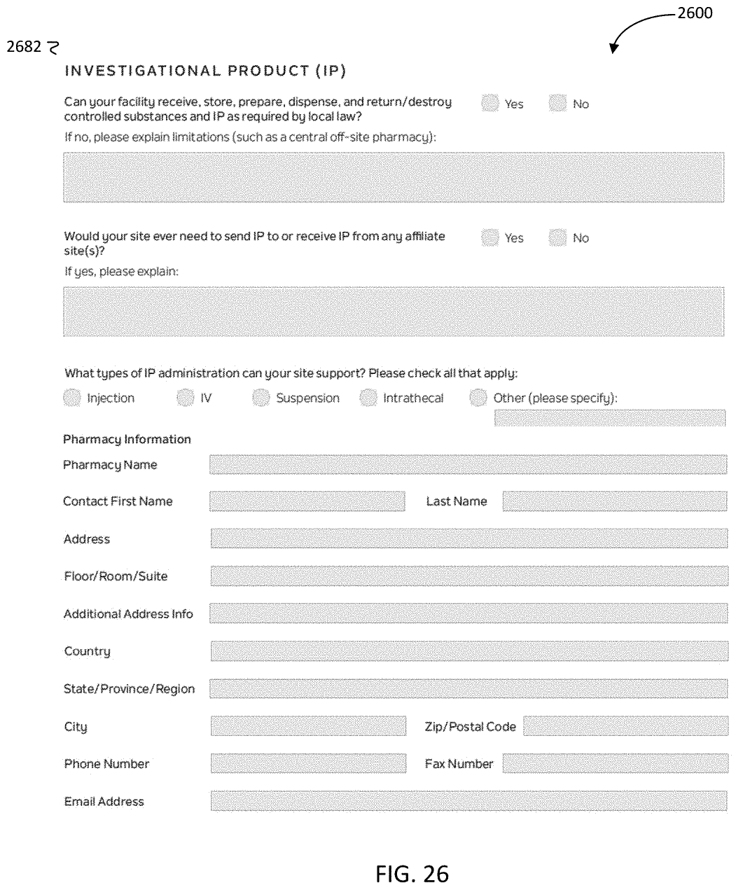

FIG. 26 is shown to include a graphical user interface (GUI) 2600. In some aspects, GUI 2600 can include investigational product (IP) 2682. In some aspects, GUI 2600 can be configured for user inputs, which can subsequently update site information within system 100.

In some aspects, IP 2682 can include handling capabilities corresponding to IP, IP administration capabilities, and/or pharmacy information.

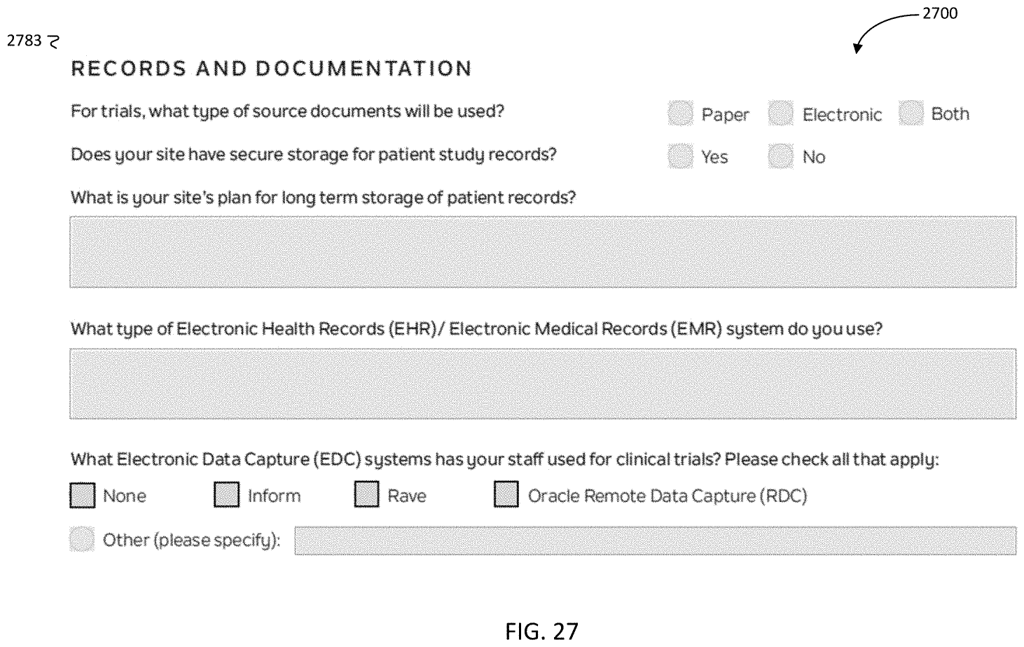

FIG. 27 is shown to include a graphical user interface (GUI) 2700. In some aspects, GUI 2700 can include records and documentation 2783. In some aspects, GUI 2700 can be configured for user inputs, which can subsequently update site information within system 100.

In some aspects, records and documentation 2783 can include source document types, record storage methods, and/or EHR/EMR systems.

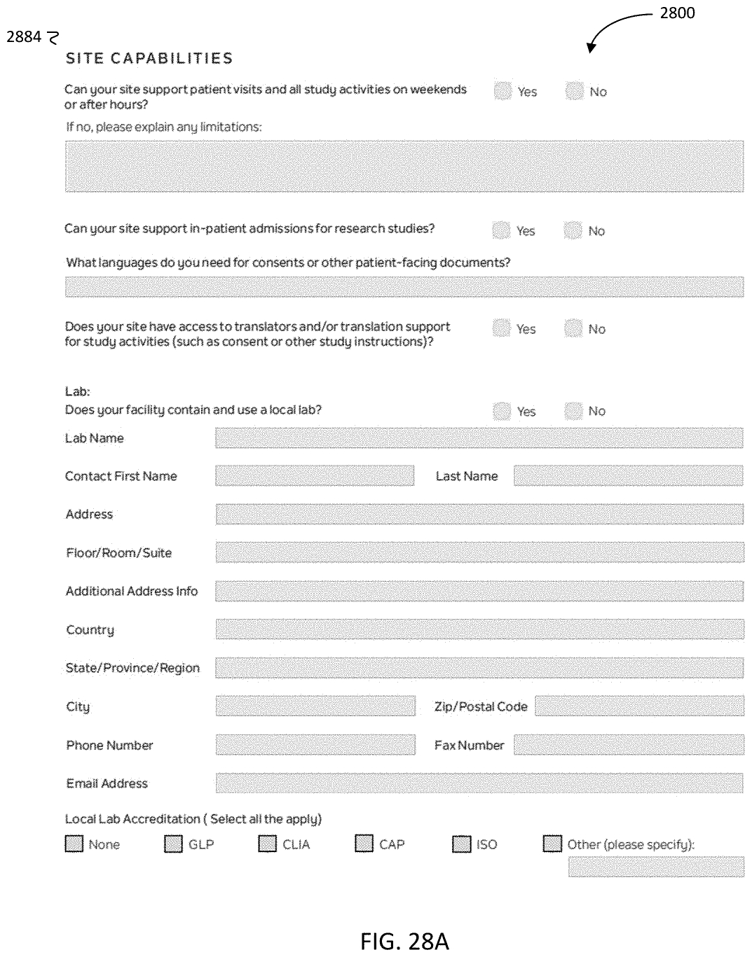

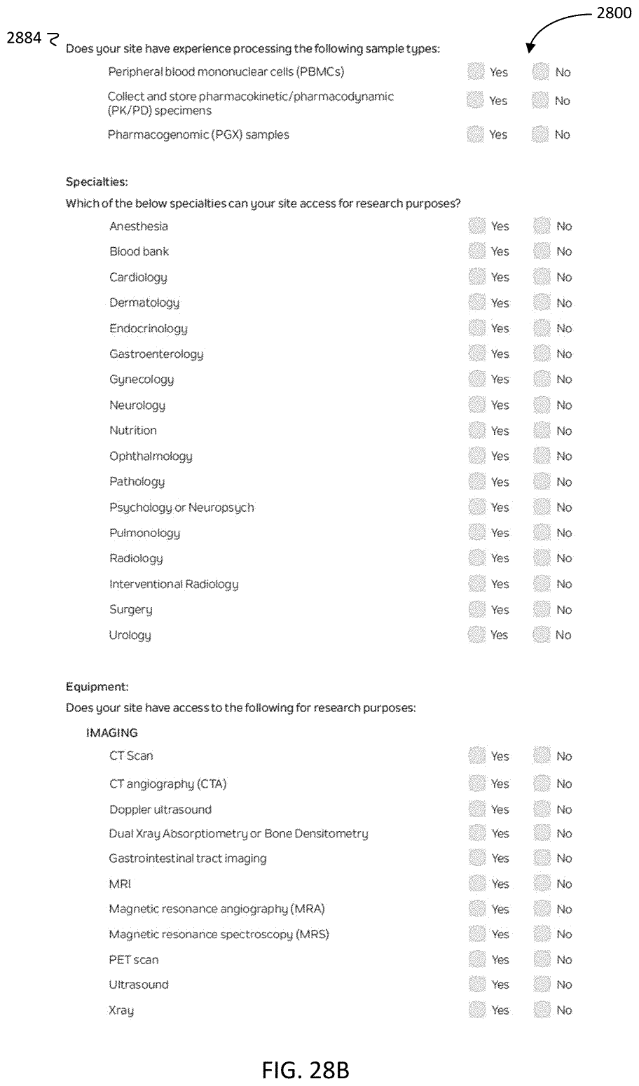

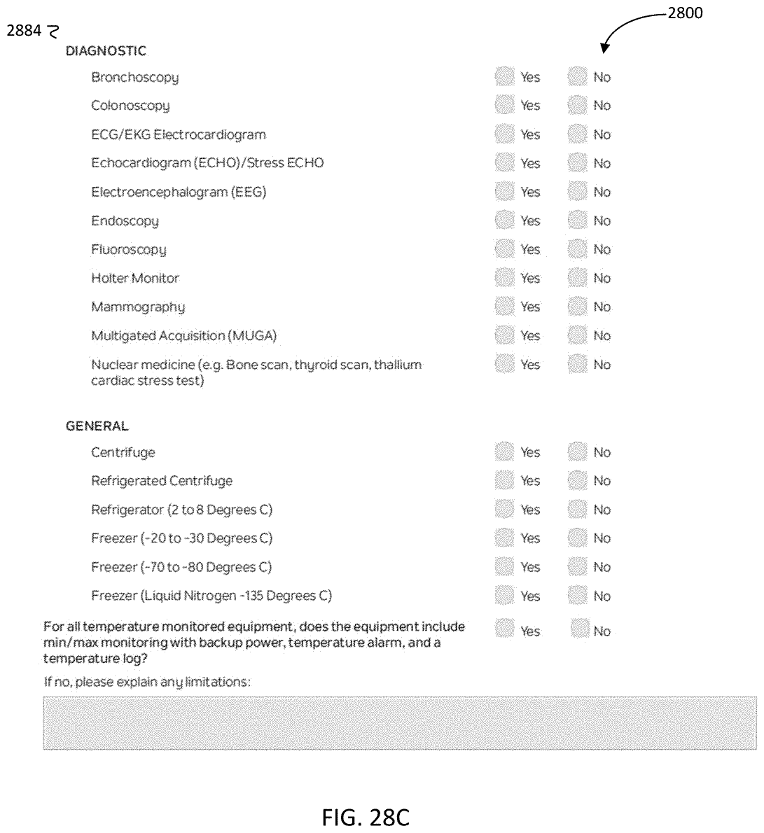

FIGS. 28A-28 C are shown to include a graphical user interface (GUI) 2800. In some aspects, GUI 2800 can include site capabilities 2884. In some aspects, GUI 2800 can be configured for user inputs, which can subsequently update site information within system 100.

In some aspects, the site capabilities 2884 can include working hours, in-patient support, language translator access, and/or local lab information. Further, the site capabilities can include specialties, equipment (e.g., imaging, diagnostic, etc.), and/or temperature monitoring capabilities.

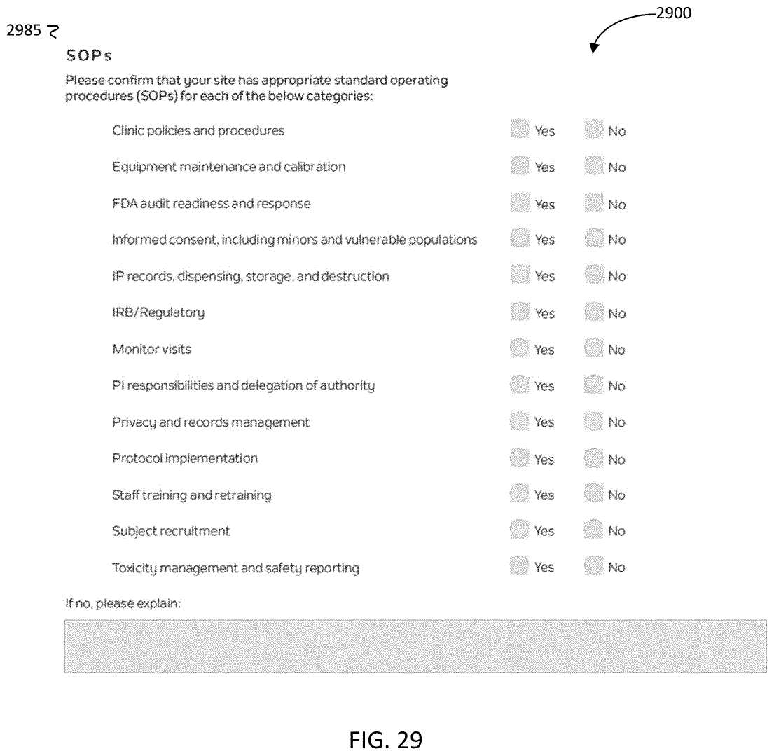

FIG. 29 is shown to include a graphical user interface (GUI) 2900. In some aspects, GUI 2900 can include standard operating procedures (SOPs) 2985. In some aspects, GUI 2900 can be configured for user inputs, which can subsequently update site information within system 100.

In some aspects, the SOPs 2985 can include FDA audit readiness, toxicity management, staff training, and/or informed consent (including minors and vulnerable populations).

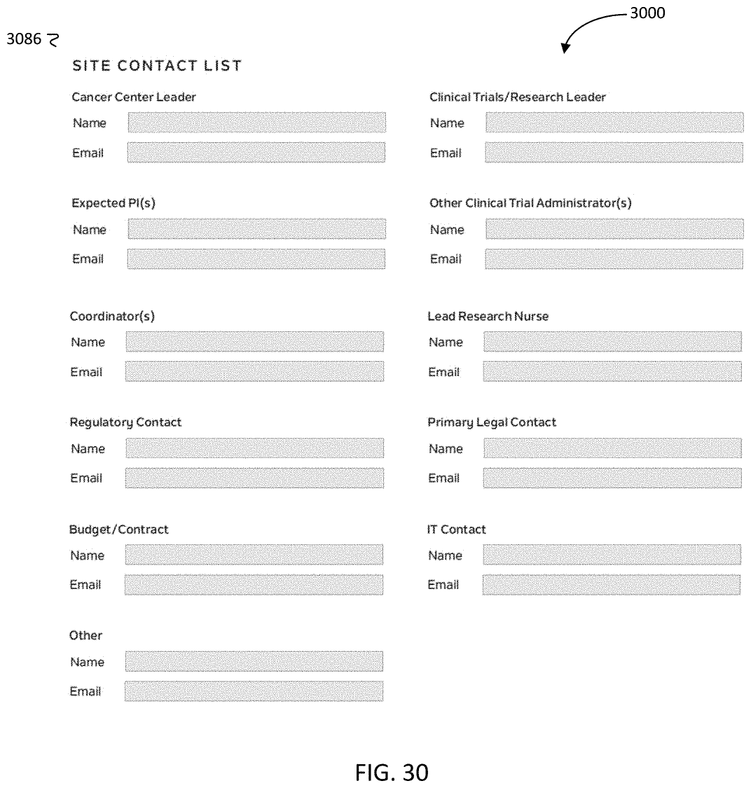

FIG. 30 is shown to include a graphical user interface (GUI) 3000. In some aspects, GUI 3000 can include a site contact list 3086. In some aspects, GUI 3000 can be configured for user inputs, which can subsequently update site information within system 100.

In some aspects, the site contact list 3086 can include information for a clinical trial leader, legal contact, regulatory contact, and/or expected PI(s).

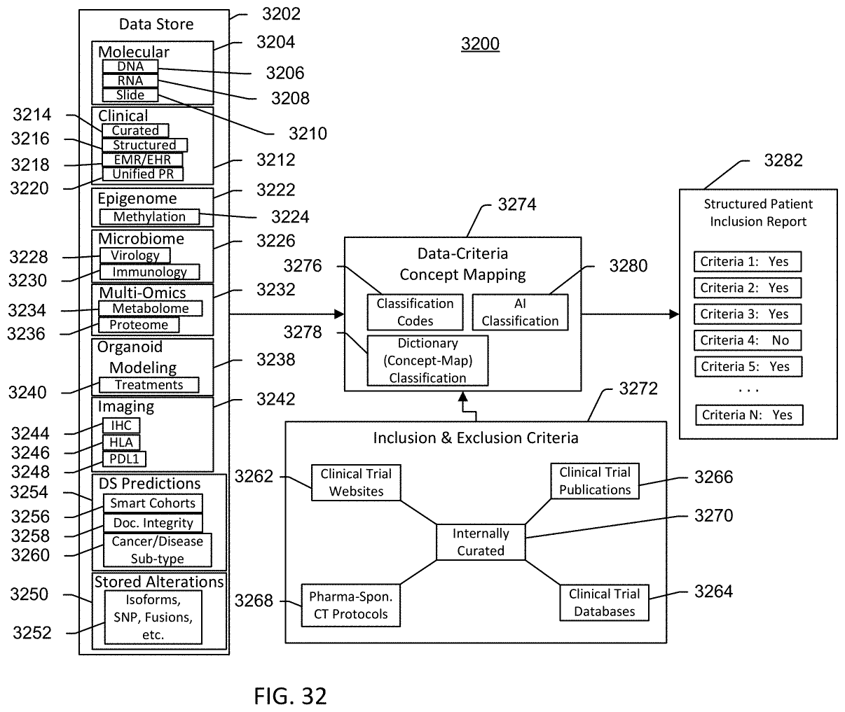

Referring now to FIG. 32, an exemplary flow 3200 for mapping clinical trial inclusion and exclusion criteria to a patient is shown. In some embodiments, the flow 3200 can match inclusion and/or exclusion criteria to patient features included in a patient data store. In some embodiments, the flow 3200 can be implemented as one or more processes and/or executed by the system 100 in FIG. 1.

In some embodiments, the flow 3200 can include a patient data store 3202. In some embodiments, the patient data store 3202 can be a database (e.g., a patient database).The patient data store 3202 can include information about a number of patients. In some embodiments, the information can include a number of features for a given patient. The features can include information related to various fields of medicine. For example, the features can include diagnoses, responses to treatment regimens, genetic profiles, clinical and phenotypic characteristics, and/or other medical, geographic, demographic, clinical, molecular, or genetic features.

In some embodiments, the flow 3200 can include generating and/or receiving a number of molecular data features 3204 for a patient. The patient data store 3202 can include the molecular data features 3204. In some embodiments, the molecular data features 3204 can be derived from RNA and/or DNA sequencing (e.g., RNA sequencing features 3206 and/or DNA sequencing features 3208), a pathologist review of stained H&E and/or IHC slides (e.g., slide features 3210), and/or further derivative features obtained from the analysis of the individual and combined results. The RNA sequencing features 3206 and/or DNA sequencing features 3208 may include genetic variants which are present in the sequenced tissue. Further analysis of the genetic variants may include additional steps such as identifying single or multiple nucleotide polymorphisms, identifying whether a variation is an insertion or deletion event, identifying loss or gain of function, identifying fusions, calculating copy number variation, calculating microsatellite instability, calculating tumor mutational burden, or other structural variations within the DNA and RNA.

In some embodiments, the flow 3200 can include generating and/or receiving slide features 3210 associated with H&E staining and/or IHC staining. For example, the slide features 3210 can include tumor infiltration, Programmed death-ligand 1 (PD-L1) Status, human leukocyte antigen (HLA) Status, and/or other immunology features can be generated based on H&E staining and/or IHC staining.

In some embodiments, the flow 3200 can include generating and/or receiving a number of clinical data features 3212 associated with the patient. The patient data store 3202 can include the clinical data features 3212. The clinical features 3212 can be derived from curated records 3214, structured records 3216, and/or electronic medical and/or health records 3218.

In some embodiments, the clinical features 3212 can include features such as diagnosis, symptoms, therapies, outcomes, patient demographics such as patient name, date of birth, gender, ethnicity, date of death, address, smoking status, diagnosis dates for cancer, illness, disease, diabetes, depression, and/or other physical or mental maladies, personal medical history, or family medical history, clinical diagnoses such as date of initial diagnosis, date of metastatic diagnosis, cancer staging, tumor characterization, tissue of origin, treatments and outcomes such as line of therapy, therapy groups, clinical trials, medications prescribed or taken, surgeries, radiotherapy, imaging, adverse effects, associated outcomes, and/or corresponding dates, and genetic testing and laboratory information such as genetic testing, performance scores, lab tests, pathology results, prognostic indicators, or corresponding dates, and/or more detailed information including date of genetic testing, testing provider used, testing method used, such as genetic sequencing method and/or gene panel, gene results, such as included genes, variants, and/or expression levels/statuses. In some embodiments, the clinical features 3212 can include a unified record database 3220. The unified record database 3220 can include copies of any of the above clinical features structured in a unified format. The unified format can allow the flow 3200 to disseminate patient features regardless of the original format the patient features were stored in, which may be helpful when matching patients from different medical systems with clinical trials.

In some embodiments, the flow 3200 can include generating and/or receiving a number of epigenome data features 3222 associated with the patient. The patient data store 3202 can include the epigenome data features 3222. In some embodiments, the epigenome data features 3222 can include methylation data features 3224.

In some embodiments, the flow 3200 can include generating and/or receiving a number of microbiome data features 3226 associated with the patient. The patient data store 3202 can include the microbiome data features 3226. In some embodiments, the microbiome data features 3226 can include virology data features 3228 and/or immunology data features 3230.

In some embodiments, the flow 3200 can include generating and/or receiving a number of multi-omic data features 3232 associated with the patient. The patient data store 3202 can include the multi-omic data features 3232. The multi-omic data features 3232 can include multi-omic features not included in the epigenome data features 3222 and/or the microbiome data features 3226. In some embodiments, the multi-omic data features 3232 can include metabolome data features 3234 and/or proteome data features 3236.

In some embodiments, the epigenome data features 3222, the microbiome data features 3226, and/or the multi-omic data features 3232 can include features derived from proteome data, transcriptome data, epigenome data, metabolome data, microbiome data, and/or other multi-omic data.

In some embodiments, the flow 3200 can include generating and/or receiving a number of organoid data features 3240 associated with the patient. The patient data store 3202 can include the organoid data features 3240. In some embodiments, the organoid data features 3240 can be generated in an organoid laboratory. In some embodiments, the organoid data features 3240 can include DNA and RNA sequencing information associated with each organoid. In some embodiments, each organoids can be associated with the patient. For example, the organoid can be generated using a tissue sample taken from the patient. In some embodiments, the organoid data features 3240 can include treatment features 3240, which may include results from treatments applied to each organoid.

In some embodiments, the flow 3200 can include generating and/or receiving a number of imaging data features 3242 associated with the patient. The patient data store 3202 can include the imaging data features 3242. In some embodiments, the imaging data features 3242 can include features derived from imaging data, such as a report associated with a stained slide, size of tumor, tumor size differentials over time (including treatments during the period of change), a classification and/or a score generated using a machine learning technique (e.g., machine learning techniques for classifying PDL1 status, HLA status, or other characteristics from imaging data). In some embodiments, the imaging data features 3242 can include an IHC slide feature 3244 (e.g., results from IHC slide analysis), an HLA feature 3246 (e.g., an HLA status), and/or a PDL1 feature 3248 (e.g., a PDL1 status).

In some embodiments, the flow 3200 can include generating and/or receiving a number of stored alteration features 3250 associated with the patient. The patient data store 3202 can include the stored alteration features 3250. In some embodiments, the stored alteration features 3250 can be generated using a machine learning technique one or more features, such as at least one of the features described above. For example, a machine learning model may generate a data science prediction, such as data science predictions 3254, of a patient's future probability of metastasis, origin of a metastasized tumor, and/or a progression-free survival probability based on a patient's state (collection of features) at any time during their treatment. In some embodiments, the stored alteration features 3250 can include features associated with Isoforms, single-nucleotide polymorphisms (SNPs), and/or Fusions.

In some embodiments, the flow 3200 can include generating and/or receiving a number of data science prediction features 3254 associated with the patient. The patient data store 3202 can include the data science prediction features 3254. In some embodiments, the data science prediction features 3254 can include a document integrity certification 3258, and/or a cancer/disease sub-type classification 3260. In some embodiments, the data science prediction features 3254 can include a number of smart cohorts 3256. In some embodiments, each of the smart cohorts 3256 can include a cohort matched to the patient based on a number of predetermined criteria such as demographics, cancer type, RNA and/or DNA mutation type, and/or any of the above features.

In some embodiments, the flow 3200 can include updating or otherwise improving features in the patient data store 3202 based on current medical research. As new testing techniques, studies, organoid screening techniques, and/or other medical improvements become available, the flow 3200 can update the features in the patient data store 3202.

some embodiments, the flow 3200 can include matching the patient with one or more clinical trials using the patient data store 3202. The patient data store 3202 can provide a number of different features as described above. The FDA requires clinical trials to register before they may enroll patients and be held. In some embodiments, the flow 3200 can include accessing registered clinical trials at one or more websites 3262, such as clinicaltrials.gov, which contains a complete listing of all clinical trials registered with the FDA. In addition to clinicaltrials.gov, the flow 3200 include accessing other government-sponsored websites and/or private websites to gather information about clinical trials. In some embodiments, the flow 3200 can include using a web crawler to periodically crawl the websites 3262 and collect information about clinical trials. The flow 3200 can add information about clinical trials to a clinical trial data storage database 3264. Clinical trials may also publish research papers identifying the clinical trial's purpose as well as any clinical trial information. In some embodiments, the flow 3200 can include curating new publications 3266 as they are published and adding the publications 3266 to the clinical trial data storage database 3264. In some embodiments, the flow 3200 can use a trained machine learning model to curate the publications 3266. In some embodiments, a medical professional can manually add publications 3266 to the clinical trial data storage database 3264.

Pharmaceutical companies and/or other institutions may maintain an institution-specific websites. The websites 3262 can include websites maintained by the pharmaceutical companies and/or other institutions. In some embodiments, the flow 3200 can include retrieving clinical trial information from one or more of the institution websites in the websites 3262. In some embodiments, the flow 3200 can include periodically querying the institution websites for clinical trial information, and adding the clinical trial information to the clinical trial data storage database 3264. Each of the websites 3262, the publications 3266, and/or the clinical trial data storage database 3264 may be treated as an independent source of clinical trial information.

Pharma-sponsored clinical trial protocols 3268 may provide detailed, dozens to hundreds of pages in reports on the detailed specifics of the clinical trial. Relationships forged between a pharmaceutical company and another partner for aggregating clinical trial information may include release of these protocols for deep learning purposes. The flow 3200 can access the pharma-sponsored clinical trial protocols 3268 to curate information from a number of different sources. The flow 3200 can compare independent sources to one another for accuracy as a whole or aggregated across each collection medium (website, publication, database, protocols), where discrepancies between sources may be evaluated by a medical professional and/or deference given to the most respected source (as a whole or in each collection medium).

In some embodiments, the flow 3200 can include routinely gathering clinical trials from the websites 3262, the publications 3266, and/or the pharma-sponsored clinical trial protocols 3268 to identify new clinical trials or modifications to existing clinical trials. In some embodiments, the flow 3200 can include adding a new clinical trial to the clinical trial data storage database 3264 and/or updating the clinical trials included in the clinical trial data storage database 3264 (e.g., as the flow 3200 encounters updates during routine web crawls).

In some embodiments, the clinical trial information can include inclusion criteria and/or exclusion criteria. The flow 3200 can map the inclusion criteria and/or exclusion criteria to the features stored in the patient data store 3202.

In some embodiments, the clinical trial information can include a study type (e.g., interventional or observational), study results, a recruitment stage (e.g., not yet recruiting, recruiting, enrollment by invitation, suspended, unknown, etc.), a title, a planned measurement such as one described in the protocol that is used to determine the effect of an intervention/treatment on participants, interventions including drugs, medical devices, procedures, vaccines, and/or other products that are either investigational or already available, interventions including noninvasive approaches of education or modifying diet and exercise, sponsors and/or funding sources, a geographic location (e.g., country, state, city, facility), a trial stage such as those based on definitions developed by the FDA for the study's objective, a number of participants, notable dates (e.g., a start date and/or an end date), and/or other characteristics (e.g., Early Phase 1, Phase 1, Phase 2, Phase 3, and Phase 4).

In some embodiments, the flow 3200 can include adding data (e.g., clinical trials and/or information associated with the clinical trials) from the websites 3262, the clinical trial data storage database 3264, the publications 3266, and/or the pharma-sponsored clinical trial protocols 3268 to an internally curated storage database 3270. The internally curated storage database 3270 can hold the criteria in the appropriate format for a data-criteria concept matching module 3274, as will be described below. To this end, specific examples of detailed clinical trial information corresponding to features stored in the patient data store 3202 and additional clinical trial information will be discussed with respect to data-criteria concept mapping below.

Features in the patient data store 3202 may be aggregated from many different sources, each source potentially having their own organizational and identification schema for structuring the features within the source. In some embodiments, the flow 3200 can include converting all incoming features to a common, structured format of the patient data store 3202. Similarly, clinical trial information may be aggregated from many different sources, each potentially having their own organizational and identification schema for structuring the clinical trial information within the source. In some embodiments, the flow 3200 can include converting all incoming clinical trial information to the common, structured format of the patient data store 3202 as well as an intermediate concept mapping to preserve inclusion and exclusion criteria in the original clinical trial information. In some embodiments, the websites 3262, the clinical trial data storage database 3264, the publications 3266, the pharma-sponsored clinical trial protocols 3268, and the internally curated storage database 3270 can be included in an inclusion and exclusion criteria module 3272.

Classification Codes for Mapping Features Between Data Stores

In some embodiments, the flow 3200 can include providing features included in the patient data store 3202 and information included in the inclusion and exclusion criteria module 3272 (e.g., inclusion criteria, exclusion criteria, clinical trial information, etc.) to the data-criteria concept matching module 3274 to match the patient to a suitable clinical trial. In some embodiments, the data-criteria concept matching module 3274 can include a classification code system 3276, a dictionary based classification system 3278, and/or an artificial intelligence (AI) classification system 3280.

In some embodiments, the classification code system 3276 can assign one or more predetermined classification codes to each feature in the patient data store 3202 and/or the corresponding inclusion/exclusion criteria in the inclusion and exclusion criteria module 3272. For example, a diagnosis of breast cancer may have a classification table. At least a portion of the classification table can include the codes in Table 1 below:

TABLE 1

| Diagnosis | Code |
|---|---|
| Breast Cancer | 63050 |
| Ductal Carcinoma In Situ | 63051 |
| Invasive Ductal Carcinomal of the Breast | 63052 |
| Tubular Carcinoma of the Breast | 63053 |
| Medullary Carcinoma of the Breast | 63054 |
| Mucinous Carcinoma of the Breast | 63055 |
| Papillary Carcinoma of the Breast | 63056 |
| Cribriform Carcinoma of the Breast | 63057 |
| Invasive Lobular Carcinoma of the Breast | 63058 |

In some embodiments, a treatment involving medications may have a classification table prioritized from brand names, chemical names, or other groupings. At least a portion of the classification table can include the codes in Tables 2A and 2B below.

TABLE 2A

| Brand (Chemical) | Code |
|---|---|
| Abraxane (albumin-bound or nab-paclitaxel) | 77121 |
| Adriamycin (doxorubicin) | 77131 |

TABLE 2B

| Chemical (Brand) | Code |
|---|---|
| Carboplatin (Paraplatin) | 78141 |
| Daunorubicin (Cerubidine, DaunoXome) | 78151 |

In some embodiments, DNA/RNA Molecular features may have a classification table for genetic mutations, variants, transcriptomes, cell lines, methods of evaluating expression (TPM, FPKM), a lab which provided the results, etc. At least a portion of the classification table can include the codes in Table 3 below.

TABLE 3

| RNA | Code |
|---|---|
| OR6C69P - Overexpressed | 1013057 |
| OR6C69P - Normal | 1013058 |
| LINC02355 - Tempus Overexpressed | 1014028 |
| LINC02355 - Foundation Overexpressed | 1014029 |
| RPS4XP15 | 1015010 |

In some embodiments, a data structure may relate the structured information as a classification code with the absolute value of the report result in a classification table. At least a portion of the classification table can include the codes in Table 4 below.

TABLE 4

| Code | Value |
|---|---|
| 1015010 | 85 TPM |
| 1015010 | 20 FPKM |

In some embodiments, inclusion and exclusion criteria may be mapped according to the same classification conventions above, however, nested criteria or more complicated criteria may be converted to another format, such as JavaScript Object Notation (JSON) to preserve the inclusion or exclusion criteria in the proper format without any information loss. For example, an inclusion criteria “Histologically or cytologically confirmed diagnosis of locally advanced or metastatic solid tumor that harbors an NTRK1/2/3, ROS1, or ALK gene rearranement” may touch Limn the following classification codes in Table 5 below.

TABLE 5

| Feature | Code |
|---|---|
| Histologically confirmed diagnosis | 20253 |
| Cytologically confirmed diagnosis | 20254 |
| Locally advanced | 20317 |
| Metastatic | 20439 |
| Solid tumor | 19001 |
| NTRK1 | 1013120 |
| NTRK2 | 1013121 |
| NTRK3 | 1013122 |
| ROS1 | 1013261 |
| ALK | 1013273 |

The inclusion criteria can be structured to represent: 19001 AND (20253 OR 20254) AND (20317 OR 20439) AND (1013120 OR 1013121 OR 1013122 OR 1013261 OR 1013273)

An inclusion criteria “At least 4 weeks must have elapsed since completion of antibody-directed therapy” may touch upon the following classification codes in a reduced-exemplary reference set in Table 6:

TABLE 6

| Feature | Code |
|---|---|
| Antibody Directed Therapy | 25001 |
| Monoclonal Antibody Therapy | 27015 |
| Nivolumab | 77233 |
| Avelumab | 77238 |
| Emapalumab | 77245 |
| Polyclonal Antibody Therapy | 27023 |
| . . . | |
| Hyperimmune Antibody Therapy | 27031 |
| . . . | |

In one example, the inclusion criteria may be structured to represent: 25001 AND (Date Administered is Older than XX/YY/ZZZZ), where all therapies which fall under Antibody Directed Therapy are assigned multiple codes, a first code 25001 for antibody directed therapy; a second code 27015, 27023, or 27031 for the type of antibody therapy, and a third code 77233, 77238, 77245 for the specific medication applied as part of the antibody therapy. In another example, the structured inclusion criteria may list all of the therapy codes which qualify in addition to 25001.

In 2016, there were 36 FDA approved monoclonal antibody therapies for the treatments of various diseases, with 17 of those for cancer. Hundreds of new therapies are currently undergoing clinical trials. Similar statistics are available for Polyclonal and hyperimmune antibody therapies. In some embodiments, each of these therapies may be listed in the above table. Each of the classification codes in Tables 1-6 can be included in the classification code system 3276.

Dictionary Classification for mapping between data stores

In some embodiments, the flow 300 can include assigning each feature in the patient data store 3202 to appropriate corresponding inclusion/exclusion criteria in the inclusion and exclusion criteria module 3272 using the dictionary based classification system 3278. The dictionary based classification system 3278 can identify relationships between features and classification codes that may not be immediately obvious. In some embodiments, the dictionary based classification system 3278 can implemented in accordance with a dictionary based classification system described in patent application Ser. No. 16/289,027 titled “MOBILE SUPPLEMENTATION, EXTRACTION, AND ANALYSIS OF HEALTH RECORDS” filed Feb. 8, 2019. In some embodiments, the dictionary based classification system 3278 can implemented in accordance with following passages of patent application Ser. No. 16/289,027, which is fully incorporated by reference:

“The process of enumerating the known drugs into a list may include identifying clinical drugs prescribed by healthcare providers, pharmaceutical companies, and research institutions. Such providers, companies, and institutions may provide reference lists of their drugs. For example, the US National Library of Medicine (NLM) publishes a Unified Medical Language System (UMLS) including a Metathesaurus having drug vocabularies including CPT®, ICD-10-CM, LOINC®, MeSH®, RxNorm, and SNOMED CT®. Each of these drug vocabularies highlights and enumerates specific collections of relevant drugs. Other institutions such as insurance companies may also publish clinical drug lists providing all drugs covered by their insurance plans. By aggregating the drug listings from each of these providers, companies, and institutions, an enumerated list of clinical drugs that is universal in nature may be generated. For example, “Tylenol” and “Tylenol 50 mg” may match in the dictionary from UMLS with a concept for “acetaminophen”. It may be necessary to explore the relationships between the identified concept from the UMLS dictionary and any other concepts of related dictionaries or the above universal dictionary. Though visualization is not required, these relationships may be visualized through a graph-based logic for following links between concepts that each specific integrated dictionary may provide.

FIG. 10 is an exemplary ontological graph database 122 for viewing links between different dictionaries (databases of concepts) that may be interlinked through a universal dictionary lookup in order to carry out the normalizing stage 70 in FIG. 5. Conventional ontological graph databases may include GraphT, Neo4j, ArangoDB, Orient, Titan, or Flockdb. The following references to dictionaries and databases are for illustrative purposes only and may not reflect accurately the concepts/synonyms, entities, or links represented therein. Links between two concepts may represent specific known relationships between those two concepts. For example, “Tylenol” may be linked to “acetaminophen” by a “trade name” marker, and may be linked to “Tylenol 50 mg” by a “dosage of” marker. There may also be markers to identify taxonomic “is a” relationships between concepts. “Is a” markers provide relationships between over some clinical dictionaries (such as SNOMEDCT_US, Campbell W S, Pederson J, etc.) to establish relationships between each database with the others. For example, we can follow “is a” relationships from “Tylenol”, “Tylenol 50 mg”, or “acetaminophen” to the concept for a generic drug. Such a relationship may not be available for another concept, for example, a match to the dictionary for UMLS to “the patient” or “patient” may not have a relationship to a medication dictionary due to the conceptually distinct natures of each entity. Relationships may be found between drugs that have the same ingredients or are used to treat the same illnesses.

Other relationships between concepts may also be represented. For example, treatments in a treatment dictionary may be related to other treatments of a separate treatment database through relationships describing the drugs administered or the illness treated. Entities (such as MMSL#3826, C0711228, RXNORM#. . . , etc.) are each linked to their respective synonyms, (such as Tylenol 50 mg, Acetaminophen, Mapap, Ofirmev, etc.). Links between concepts (synonyms), may be explored to effectively normalize any matched candidate concept to an RXNORM entity.

Returning to FIG. 10, the concept candidate “Tylenol 50 mg” 124 may have a hit in the National Library of Medicine Database MMSL. In the preceding stage of the pipeline, “Tylenol 50 mg” may have been linked to the Entity MMSL#3826 126 as an identifier for the “Tylenol 50 mg” concept in MMSL. The linked Entity, MMSL#3826, may reside in a database which is not a defined database of authority, or, for document classification purposes, MMSL#3826 may not provide a requisite degree of certainty or provide a substantial reference point needed for document/patient classification. Through entity normalization, it may be necessary to explore links to MMSL#3826 until a reference entity of sufficient quality is identified. For example, the RXNORM database may be the preferred authority for identifying a prescription when classifying prescriptions a patient has taken because it provides the most specific references to drugs which are approved by the U.S. Food and Drug Administration (FDA).

Other authorities may be selected as the normalization authority based upon any number of criteria. The exact string/phrase “Tylenol 50 mg” may not have a concept/entity match to the RXNORM database and the applied fuzzy matching may not generate a match with a high degree of certainty. By exploring the links from MMSL#3826, it may be that concept “Tylenol Caplet Extra Strength, 50 mg” 128 is a synonym to “Tylenol 50 mg” in the MMSL database. Furthermore, concept “Tylenol Caplet Extra Strength, 50 mg” may also be linked to Entity C0711228 130 of the UMLS database. By exploring the synonyms to “Tylenol 50 mg” 124 through Entity MMSL#3826 126, the concept candidate may be linked to the UMLS Entity C0711228 130. However, the UMLS Entity C0711228 130 is not the preferred authority for linking prescriptions, so further normalization steps may be taken to link to the RXNORM database. Entity C0711228 130 may have synonym “Tylenol 50 MG Oral Tablet” 132 which is also linked to RXNORM#5627 134. RXNORM#5627 134 may be a normalization endpoint (once RXNORM#5627 has been identified, normalization may conclude); however, RXNORM#5627 134 may also represent the Tylenol specific brand name rather than the generic drug name. A degree of specificity may be placed for each source of authority (normalization authority) identifying criteria which may been desired for any normalized entity. For example, a medication may need to provide both a brand drug name and a generic drug name. Links in the RXNORM database may be explored to identify the Entity for the generic drug version of Tylenol. For example, RXNORM#5627 134 may have an “ingredient of link to RXNORM#2378 136 which has a “has tradename” link to RXNORM#4459 138 with concept acetaminophen. RXNORM#4459 138 is the Entity within the RXNORM database which represents the generic drug 140 for Tylenol 50 mg and is selected as the normalized Entity for identifying a prescription in the classification of prescriptions a patient has taken. In this aspect, normalization may first identify an Entity in the dictionary of authority (as defined above) and may further normalize within the dictionary of authority to a degree of specificity before concluding normalization.”

The dictionary based classification system 3278 can curate inclusion and exclusion criteria using a well-defined clinical/ontological dictionary to provide classifications based upon language concepts rather than codes. In some embodiments, the flow 3200 can include using the classification codes 3276 and the dictionary based classification system 3278 to use concept-based classification to map features and/or criteria to an internal code index. In some embodiments, the dictionary based classification system 3278 can output whether or not inclusion criteria and/or exclusion criteria in the inclusion and exclusion criteria module 3272 based on features in the patient data store 3202.

### Artificial Intelligence for Predicting Patient Eligibility for Clinical Trials or Criteria

In some embodiments, the AI classification system 3280 can include at least one trained model that can receive inclusion criteria and/or exclusion criteria in the inclusion and exclusion criteria module 3272 and features in the patient data store 3202, and output at least one indication of whether or not at least one criteria is met or not met. In some embodiments, the trained model can be a neural network or other appropriate machine learning model trained on a training data set. For a data-criteria concept mapping classifier, an exemplary training data set may include patient information (e.g., features that may be included in the patient data store 3202), clinical trial information including inclusion and exclusion criteria (e.g., criteria that may be included in the inclusion and exclusion criteria module 3272), and resulting line-by-line classification results for whether the inclusion or exclusion criteria were met (e.g., ground truths).

In some embodiments, the model(s) can include supervised algorithms (such as algorithms where the features/classifications in the data set are annotated) using linear regression, logistic regression, decision trees, classification and regression trees, Naive Bayes, nearest neighbor clustering; unsupervised algorithms (such as algorithms where no features/classification in the data set are annotated) using Apriori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines.

NNs include conditional random fields, convolutional neural networks, attention based neural networks, long short term memory networks, or other neural models where the training data set includes a plurality of tumor samples, RNA expression data for each sample, and pathology reports covering imaging data for each sample. While MLA and neural networks identify distinct approaches to machine learning, the terms may be used interchangeably herein. Thus, a mention of MLA may include a corresponding NN or a mention of NN may include a corresponding MLA unless explicitly stated otherwise. Artificial NNs are efficient computing models which have shown their strengths in solving hard problems in artificial intelligence. They have also been shown to be universal approximators (can represent a wide variety of functions when given appropriate parameters). One of the major criticisms for NNs, is their being black boxes, since satisfactory explanation of their behavior may be difficult to discern. While research is ongoing to pierce the veil of NN learning, the rules driving the classification process are usually, and may continue to be, indecipherable black boxes. Similar constraints exist for some, but not all MLA. For example, some MLA may identify features of importance and identify a coefficient, or weight, to them. The coefficient may be multiplied with the occurrence frequency of the feature to generate a score, and once the scores of one or more features exceed a threshold, certain classifications may be predicted by the MLA. A coefficient schema may be combined with a rule based schema to generate more complicated predictions, such as predictions based upon multiple features. For example, ten key features may be identified across three different classifications. A list of coefficients may exist for the features, and a rule set may exist for the classification. A rule set may be based upon the number of occurrences of the feature, the scaled weights of the features, or other qualitative and quantitative assessments of features encoded in logic known to those of ordinary skill in the art. In other MLA, features may be organized in a binary tree structure. For example, key features which distinguish between the most classifications may exist as the root of the binary tree and each subsequent branch in the tree until a classification may be awarded based upon reaching a terminal node of the tree. For example, a binary tree may have a root node which tests for a first feature. The occurrence or non-occurrence of this feature must exist (the binary decision), and the logic may traverse the branch which is true for the item being classified. Additional rules may be based upon thresholds, ranges, or other qualitative and quantitative tests.

While supervised methods are useful when the training dataset has many known values or annotations, the nature of EMR/EHR documents is that there may not be many annotations provided. When exploring large amounts of unlabeled data, unsupervised methods are useful for binning/bucketing instances in the data set. Returning to the example regarding gender, an unsupervised approach may attempt to identify a natural divide of documents into two groups without explicitly taking gender into account. On the other hand, a drawback to a purely unsupervised approach is that there's no guarantee that the division identified is related to gender. For example, the division may be between patients who went to Hospital System A and those who did not rather than the desired division.

In some embodiments, the data-criteria concept matching module 3274 can include a number of trained models, each trained model being associated with a specific inclusion criteria or exclusion criteria. For example, each trained model can receive at least one feature and output and indication whether the criteria is met or not met. Abstraction and Valuesets for Inclusion/Exclusion Criteria Templates

In some embodiments, at least a portion of the features in the patient data store 3202 can be populated using an abstraction technique. In some embodiments, the abstraction technique can include a process including providing and/or displaying a medical document associated with a patient to a medical abstractor (e.g., a person trained to disseminate medical documents), receiving at least one feature (e.g., a feature generated by the abstractor), and adding the at least one feature to the patient data store 3202.

The features of the patient data store 3202 can be aggregated from millions of documents across thousands of sources. Thus, it may be practically impossible for an abstractor to keep in mind all the types of features that may be extracted from any particular document from any particular source. An abstraction software suite may be programmed or utilize a trained artificial intelligence to recognize a document type from a source and extract all relevant information from the document and storing a digital representation in a structured format according to the above disclosure.

In some embodiments, an AI technique may not be able to make a complete abstraction from any document, or may encounter a new document or document in such bad condition that optical character recognition is not available which renders automatic abstraction ineffective. A software suite that is aware of data elements corresponding to the type of fields commonly found in medical documents may enable an abstractor to systematically convert information from the document into the structured format required in the mapping process.

For example, a document may have patient information from a next generation sequencing report containing molecular marker results or laboratory testing results from testing performed on a patient's blood. Standard information, such as the patient's name, date of birth, address may be found in a document. Other information such as the laboratory name, address, CAP/CLIA number, testing procedure performed may also be present. Clinical information such as the results of the next generation sequencing test, such as specific single nucleotide variants, copy number alterations, fusions, or other genomic alterations may be reported.

A well-informed abstraction suite may inclde valuesets for each type of information that may be found in the document. A patient valueset may contain fields for patient name (text), date of birth (date), address (structured text for street, apartment or suite number, city, state, zip code), or other patient information. A Laboratory valueset may contain fields for lab name (text), address (structured text for street, apartment or suite number, city, state, zip code), requesting institution name or address, requesting physician name, testing requested (blood test, sequencing of tissue, etc), and particular results from the test, such as: blood test [blood type, White blood count, red blood count, bilirubin count, etc], sequencing results [gene name, gene expression, variants detected, etc]. Each field may further be identified by the units of the field, for example, as shown below, absolute neutrophil count may be measured by “10³ Cells per microLiter (CPμL)”, “10³ Cells per microLiter (CPμL)”, or “K/mm³ (KMM)” which are equivalent measurements across differing institutions. A dropdown may allow an abstractor to identify the units which relate to the field that is populated.

Example data elements or fields that an abstractor may find in a respective template may be mapped to respective inclusion/exclusion criteria according the below tables.

```
TABLE 7
Inclusion Criteria Mapping
Total bilirubin >= 1.5 × institutional upper limit of normal (ULN)
  Bilirubin Count (bCnt)
  Greater than equal to (GTET)
  1.5
```

| | |
|---|---|
| Institution ID (HD) | Physician ID (pID) |
| Institutional ULN (iULN) | Physician ULN (pULN) |
| Inclusion Expression(s): | |

Binary (T/F) = bCnt >= 1.5 × (iULN(iID))
Binary (T/F) = bCnt >= 1.5 × (pULN(pID))
Binary (T/F) = bCnt >= 1.5 × (iULN(iID,pID))

In a template for mapping bilirubin count to an inclusion criteria, a phrase “Total bilirubin >=1.5×institutional upper limit of normal (ULN)” may be parsed from a clinical trial inclusion/exclusion criteria document into a series of data elements that must be present, and then an expression may be generated which represents the criteria in a computer calculable algorithm which maps the requisite data elements top to their respective values along with the expected mathematical expressions used to generate the result. A binary, true/false or yes/no may be generated using the expressions. In the abstraction software suite, an abstractor may abstract from a report containing details of a laboratory blood test. The template may prompt the abstractor for patient information which links the patient to the rest of the information, the template may further prompt the abstractor for an institution or laboratory that performed the test as well as an ordering institution and/or physician if available. For immutable values, an institution or physician repository may exist for storing constants such as the institutional upper limit of normal (iULN) or physician specific upper limit of normal (pULN). In this way, data elements which may act as equivalent representations may share the same row (such as ilD and pID) where unique data elements receive their own rows. The abstractor may be able to populate such immutable values in the template or the abstraction software may automatically retrieve such values from the corresponding repository. For other values, the abstractor may insert the value into the respective field of the abstraction template. The inclusion criteria may be stored in a structured format once each of the data elements are extracted and the relationships between them preserved. Each inclusion expression may be stored by a code ID or in a form of overloaded function which has optional arguments which may be populated to select the correct expression.

TABLE 8
Inclusion Criteria Mapping
Aspartate Aminotransferase (AST)/Serum Glutamic-Oxaloacetic
Transaminase (SGOT) >= 1.5 × institutional upper limit of normal (ULN)

| | |
|---|---|
| AST Count (astCnt) | SGOT Count (sgotCnt) |
| Less than equal to (LTET) | |
| 2.5 | |
| Institution ID (HD) | Physician ID (pID) |
| Institutional ULN (iULN) | Physician ULN (pULN) |
| Inclusion Expression(s): | |

Binary (T/F) = astCnt <= 2.5 × (iULN(iID))
Binary (T/F) = astCnt <= 2.5 × (pULN(pID))
Binary (T/F) = astCnt <= 2.5 × (iULN(iID,pID))

A second example for AST is detailed above.

```
TABLE 9
Exclusion Criteria Mapping
Absolute neutrophil count (ANC) >= 1.5 × 10⁹/L
  ANC Count (ancCnt)
  Greater than equal to (GTET)
  1.5
```

| | | |
|---|---|---|
| 10⁹ Cells per Liter (CPL) | 10³ Cells per | K/mm³ (KMM) |
| | microLiter (CPμL) | |
| Exclusion Expression(s): | | |

Binary (T/F) = ancCnt(CPL) >= 1.5 × (1,000,000,000)
Binary (T/F) = ancCnt(CPμL) >= 1.5 × (1,000)
Binary (T/F) = ancCnt(KMM) >= 1.5 × (1,000)

A final example for an exclusion criteria based upon ANC is above.

As an abstractor populates entries in the abstraction software suite, an abstraction system may begin mapping which clinical trials may be informed by either keeping a tally of which data elements have been populating and comparing that to a table of data elements required per study (clinical trial), or other data curation schema. For example, a abstraction system may poll new abstraction entries for each patient, identify new data elements populated in the newest document, and re-evaluate patient's eligibility across all of the available clinical trials. This may be performed by using a table with every clinical trial (study) having its own row, where the each inclusion or exclusion expression is given a row, the cell where each row and column meet contains information on whether the study requires satisfaction of the expression (T), fails satisfaction of the expression (F), or does not require the expression (Null). If a patient satisfies the expression for all (T) and does not satisfy the expression for all (F), then they are indicated as eligible for the associated clinical trial.

TABLE 10

| | Data Elements | | | |
|---|---|---|---|---|
| | bCnt >= 1.5 × | astCnt <= 2.5 × | ancCnt(KMM) >= | . . . |
| | (iULN(iID)) | (iULN(i1D)) | 1.5 × (1,000) | |
| Study 1 | T | Null | F | . . . |
| Study 2 | F | F | T | . . . |
| Study 3 | Null | T | F | . . . |
| . . . | . . . | . . . | . . . | . . . |

Additionally, the data elements may be separated into a requirements table and a calculations table such that a study is only considered once all data elements that appear in the study's inclusion/exclusion criteria have been satisfied. Even further, data elements may be split into static and temporal classifications where a static classification is a data element that is not expected to change over time (gender, cancer site, previous treatments received, etc) and temporal classification is a data element that is subject to change (age, treatments not yet received, metastasis, smoking, blood pressure, white/red blood cell counts, etc). A patient may be recommended as potentially eligible for a clinical trial once the static classifications are all met, and the patient may be informed of the temporal classifications which need to be met. In this manner, a patient who would otherwise be eligible for a clinical trial, except that they have not had a blood test performed in the last six months may be informed that pending the results of a blood test, they may be eligible for the clinical trial. Thusly, encouraging the patient to consider getting a blood test to make their patient record more robust and potentially entering into an applicable clinical trial.

In some embodiments, institutions or patients may opt into an automatic notification system which allows clinical trials to regularly query for applicable patients, set up reoccuring queries for eligible patients, or receive real time alerts when a patient has satisfied the criteria so that they may request the patient's participation.

TABLE 11

| | Data Elements | | | | | | | |
|---|---|---|---|---|---|---|---|---|
| | | | ancCnt | | | | | |
| | bCnt | astCnt | (KMM) | iID | pID | iULN | pULN | . . . |
| Study 1 | Y | Y | N | Y | Y | Y | Y | . . . |
| Study 2 | Y | Y | N | Y | Y | Y | Y | . . . |
| Study 3 | N | N | Y | N | N | N | N | . . . |
| . . . | . . . | . . . | . . . | . . . | . . . | . . . | . . . | . . . |

TABLE 12

| | Data Elements | | | |
|---|---|---|---|---|
| | bCnt | astCnt | ancCnt(KMM) | . . . |
| Study 1 | >= 1.5 × (iULN(iID)) | <= 2.5 × (iULN(iID)) | Null | . . . |
| Study 2 | >= 2.5 × (iULN(iID)) | Null | >= 1.5 × (1,000) | . . . |
| Study 3 | Null | <= 5 × (iULN(iID)) | >= 3 × (1,000) | . . . |
| . . . | . . . | . . . | . . . | . . . |

The flow 3200 can include generating a report 3282 for a patient with respect to any clinical trial. In some embodiments, the report 3282 can be a structured patient inclusion report. In some embodiments, the report 3282 may list the inclusion and exclusion criteria for a clinical trial and an indication of whether the patient satisfies the criteria. In some embodiments, the indication can be in the form of a written result or may be presented as or in combination with a color code such as green for satisfying or red for failing each criteria. The flow 3200 can generate the report 3282 for qualifying clinical trials which are relevant to a patient and provided to the patient's physician for discussion with the patient.

In some embodiments, the flow 3200 can include generating the report 3282 at predetermined time point and/or as new information about a patient or trial becomes available. For example, the flow 3200 can include generating the report 3282 at regular time points (e.g., daily), in response to the clinical trial information being updated (e.g., in response to detecting that the clinical trial information has been updated) and/or in response to the patient data store 3202 being updated (e.g., in response to detecting that the patient data store 3202 has been updated). Through the use of validation contracts that represent clinical trialprotocol inclusion & exclusion criteria, programmatic and automated evaluation of a patient's eligibility for any given clinical trial can be evaluated.

In some embodiments, the validation contracts can be altered/managed and run either on-demand or automatically. Further, patient data being evaluated may be sourced from either/all of the patient data store 3202 components (e.g., the curated records 3214, the structured records 3216, the electronic medical and/or the health records 3218, the multi-omic data features 3232, etc.).

In some embodiments, the validation contracts can be used to help identify patients eligible for a trial (rather than a specific patient's eligibility for a trial). In these scenarios, patient content can be transmitted and processed in real-time, generating data products that include pertinent patient data that fall within acceptable and permissible inclusion/exclusion criteria.

In some embodiments, the validation contracts can be used to help predict the feasibility of filling and completing enrollment for a given clinical trial protocol based on prior observed incidences of similar patient attributes across the data store components (e.g. the curated records 3214, the structured records 3216, the electronic medical and/or the health records 3218, the multi-omic data features 3232, etc.). The feasibility of filling and completing enrollment analysis can be included in the report 3282 and/or a separate report.

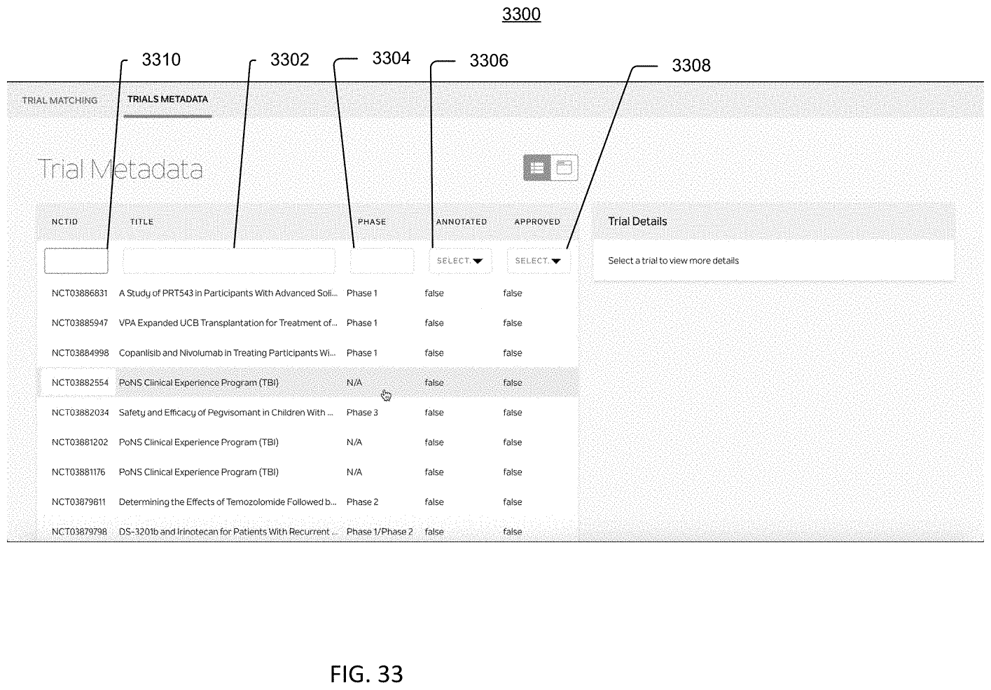

FIG. 33 is shown to include a graphical user interface (GUI) 3300. In some embodiments, GUI 3300 can be implemented by the system 100 in FIG. 1. In some embodiments, the GUI 3300 can be included in a trial metadata application. The trial metadata application can extract information from trial sources (e.g., clinicaltrials.gov) and/or convert the information into a unified format (e.g., data structures). Some clinical trials may be publicly available (e.g., available at clinicaltrials.gov). Some clinical trials may be private (e.g., a pharmaceutical company trial). The GUI 3300 can include a number of searchable fields, such as an NCTID field 3310, a title field 3302, a phase field 3304, an annotated field 3306, and/or an approved field 3308. The GUI 3300 can be used (e.g., by an abstractor) to look up clinical trials in order to review and/or populate extracted data fields. The annotated field 3306 can indicated if an abstractor has finished annotating (e.g., extracting data fields) the clinical trial, and the approved field 3308 can indicate if a reviewer (e.g., a second abstractor and/or oncologist) has finished reviewing the extracted data fields. Once approved, the clinical trial can be entered into a searchable database of clinical trials.

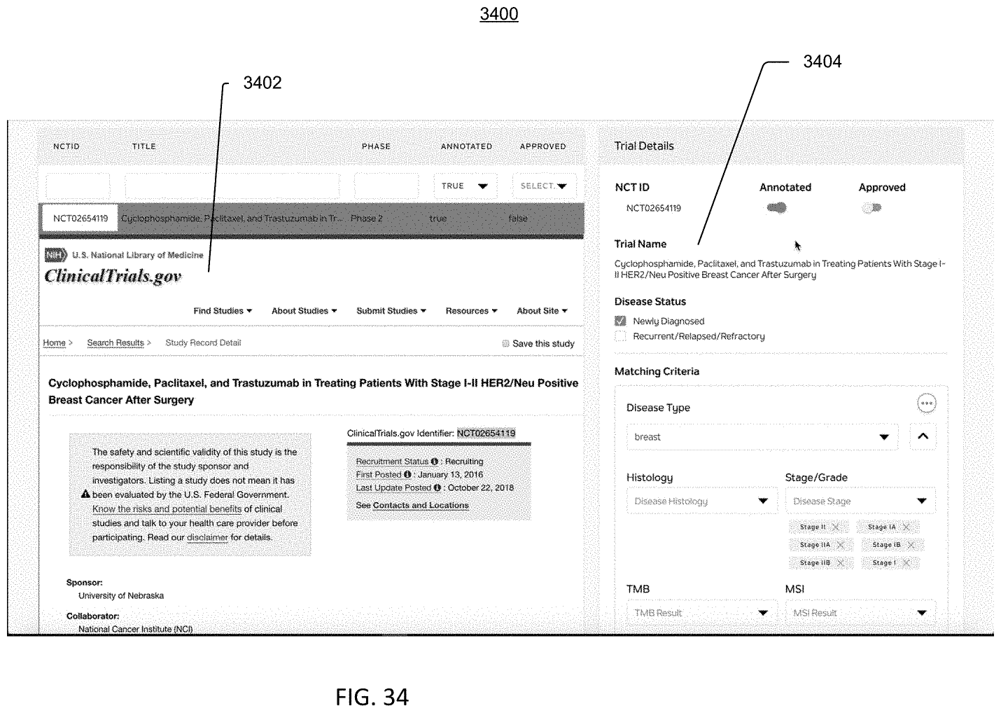

FIG. 34 is shown to include a graphical user interface (GUI) 3400. In some embodiments, GUI 3400 can be implemented by the system 100 in FIG. 1. In some embodiments, the GUI 3400 can include a clinical trial source portion 3402 and a data field portion 3404. The clinical trial source portion 3402 can display the original source (e.g., a webpage) of information about the clinical trial. Typically, the original source may include the information that is unstructured (e.g., free text) and/or may use terminology that does not align exactly with a standardized set of medical terms (e.g., SNOMED). The GUI 3400 can display the clinical trial source portion 3402 to an abstractor, who can interact with the data field portion 3404 in order to produce a number of data fields. The data fields in the data field portion 3404 can follow a standardized terminology and/or a standardized formatting style, which can allow the clinical trial to be searched and/or matched to a patient. The data fields can include a number of inclusion criteria and/or exclusion criteria, which may be included in the inclusion and exclusion criteria module 3272 in FIG. 32. The data fields can also include a number of logistical details about the clinical trial (e.g., funding source, organizer, location, etc.). While the logistical details may not be necessary for the inclusion criteria or exclusion criteria for the clinical trial, the logistical details can help match a patient to a relevant clinical trial (e.g., a trial within one hour of driving). In some embodiments, the clinical trial source portion 3402 and the data field portion 3404 can be arranged in parallel to each other in order to increase abstractor efficiency, as the abstractor can view the original trial information and enter formatted data on the same page.

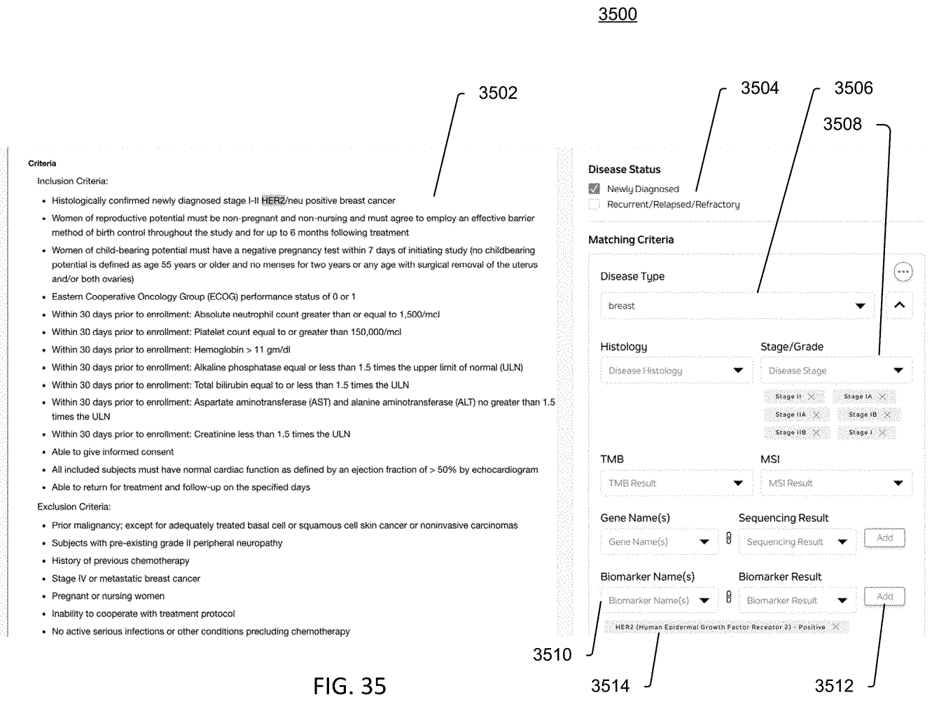

FIG. 35 is shown to include a graphical user interface (GUI) 3500. In some embodiments, GUI 3500 can be implemented by the system 100 in FIG. 1. In some embodiments, the GUI 3500 can include a clinical trial source portion 3502 and a data field portion 3504. The data field portion 3504 can include a disease type field 3506. The disease type field 3506 can be a cancer (e.g., breast cancer). The data field portion 3504 can include a stage/grade field 3508. In some embodiments, multiple stages/grade options can be selected. For example, the multiple stages can be populated based on clinical trial source information in the clinical trial source portion 3502. It is important to include a large number of option for certain data fields, which may have hundreds of options, because clinical trials can very granular in selecting inclusion/exclusion criteria. The data field portion 3504 can include a biomarker name field 3510. The biomarker name field 3510 can include hundreds of biomarkers (e.g., HER2-Positive). The abstractor can select a biomarker name (e.g., using a drop-down at the biomarker name field 3510), and add the biomarker name field 3510 using an add element 3512. A filter 3514 corresponding to the selected biomarker name can then be presented on the GUI 3500.

In some embodiments, at least a portion of data fields in the data field portion 3504 can be structured data fields from pre-existing medical lexicons. In this way, the GUI 3500 can map the “free text” in the clinical trial source portion 3502 to standardized fields. In some embodiments, the types of structured data fields can include data fields used in EMRs, data fields used in a database maintained by a medical organization (e.g., a university, a private company, a hospital system, etc.), data fields used in electronic data warehouses, and/or other structured data fields.

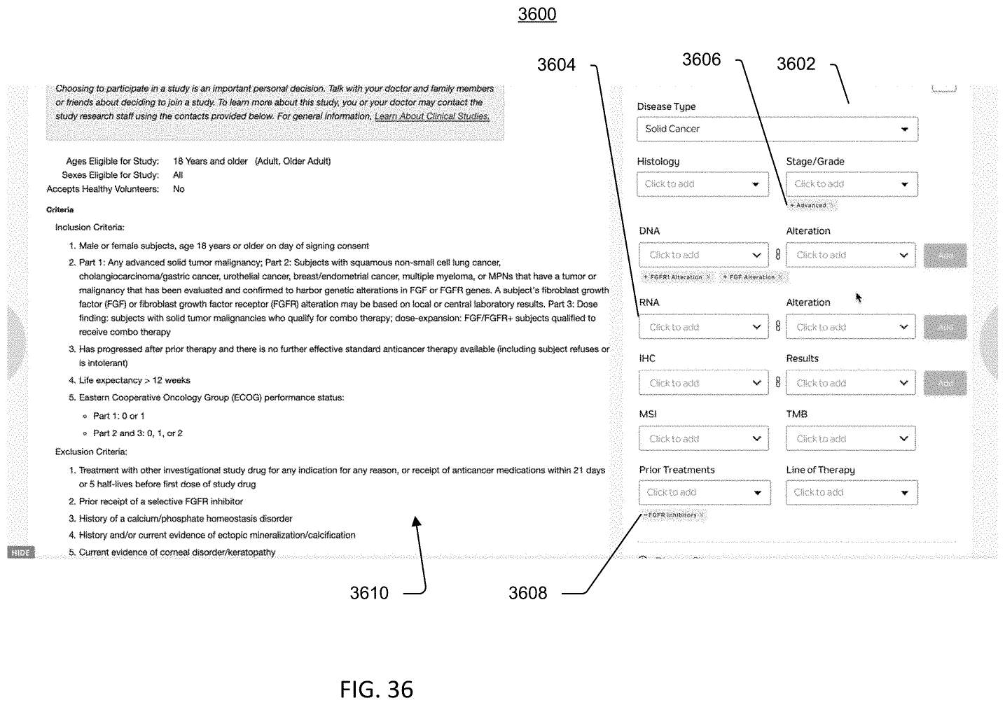

FIG. 36 is shown to include a graphical user interface (GUI) 3600. In some embodiments, GUI 3600 can be implemented by the system 100 in FIG. 1. In some embodiments, the GUI 3600 can include a data field portion 3602. The data field portion 3602 can include a number of data fields, which can be removed or added as needed, for example, by an abstractor. The data field portion 3602 can include an RNA field 3604. As an example, some clinical trials may not use RNA as inclusion criteria and/or exclusion criteria. Thus, an abstractor may choose to remove the RNA field 3604 in order to “clean up” the data field portion 3602. The data field portion 3602 can include a number of filters, such as a stage/grade filter 3606 and a prior treatments filter 3608. Some of the filters, such as the stage/grade filter 3606, can be positive filters that can act as inclusion criteria. Some of the filters, such as the prior treatments filter 3608, can be negative filters that can act as exclusion criteria.

In some embodiments, a natural language processor (NLP) can pre-populate the data field portion 3602 with a number of data fields and/or filters based on clinical trial source information. The GUI 3600 can include a clinical trial source portion 3610. The NLP can ingest at least a portion of the clinical trial source portion 3610 and populate the data field portion 3602 with a number of suitable data fields and/or filters.

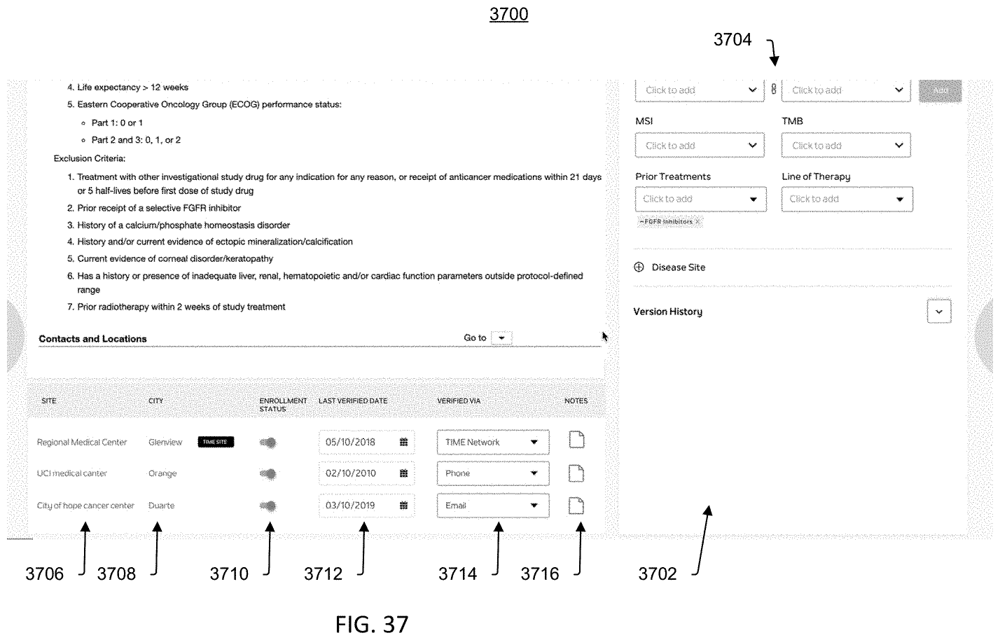

FIG. 37 is shown to include a graphical user interface (GUI) 3700. In some embodiments, GUI 3700 can be implemented by the system 100 in FIG. 1. In some embodiments, the GUI 3700 can include a version history 3702 that includes a list of changes made to a data field portion 3704. In some embodiments, the list can include a user ID (e.g., an abstractor name), a timestamp of the change, and a summary of the change (e.g., which data fields and/or filters were modified, changes to an annotated status or an approved status, etc.).

In some embodiments, the GUI 3700 can include a trial information portion. The trial information portion can include logistical information about the clinical trial. In some embodiments, the trial information portion can include a number of site fields 3706. For each site field 3706 in the number of site fields, the GUI 3700 can include a city field 3708, an enrollment status field 3710, a last verified date field 3712, a verification source field 3714, and/or a notes field 3716. The last verified data field can indicate the most recent time the city field 3708 and the enrollment status field 3710 were verified, and the verification source field 3714 can indicate the source used to verify the city field 3708 and the enrollment status field 3710 (e.g., a website, phone contact with a trial organizer, an email with a trial organizer, etc.). The notes field 3716 can include supplemental materials about the clinical trial and/or the location of the trial as indicated in the corresponding site field 3706. In some embodiments, the site field 3706, the city field 3708, the enrollment status field 3710, the last verified date field 3712, the verification source field 3714, and/or the notes field 3716 can be updated by an external source such as the site hosting the trial. For example, a site organizer update the enrollment status field 3710, the last verified date field 3712, the verification source field 3714, and/or the notes field 3716 using a suitable application, which can keep the information about the trial up to date.

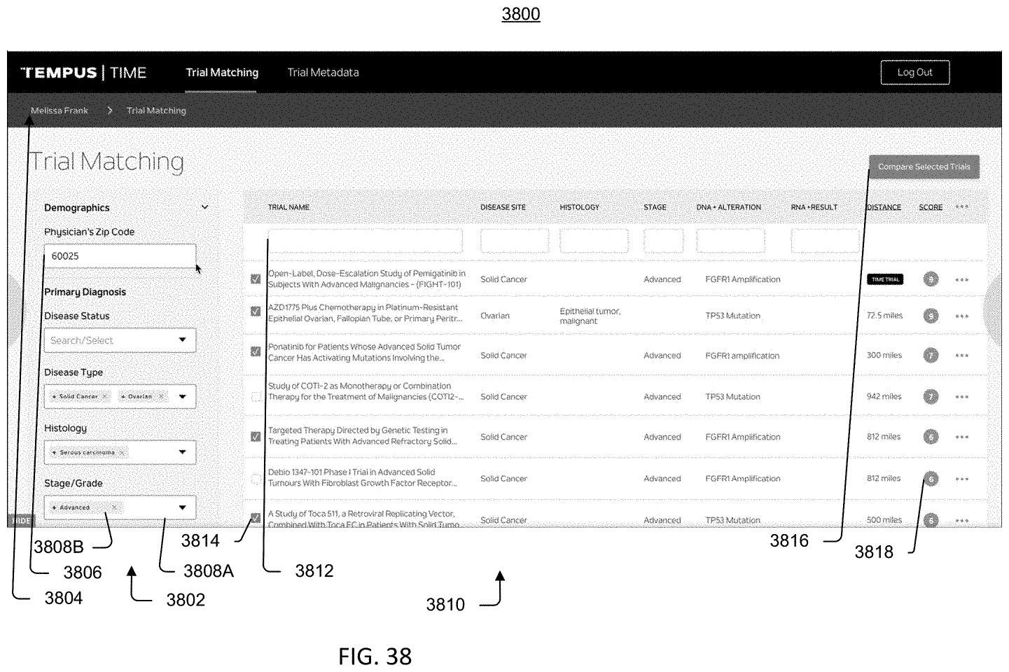

FIG. 38 is shown to include a graphical user interface (GUI) 3800. In some embodiments, GUI 3800 can be implemented by the system 100 in FIG. 1. In some embodiments, the GUI 3800 can include a search parameter portion 3802 and a search results portion 3810. The search parameter portion 3802 can include a number of search parameter fields that a user (e.g., a physician) can populate in order to find a relevant clinical trial for a patient. In some embodiments, a patient name 3804 can be included in the GUI 380. In some embodiments, the search parameter portion 3802 can include a location field 3806 (e.g., a zip code field), a stage/grade field 3808 A, and/or other suitable fields. In some embodiments, certain fields can be populated with multiple data values. In some embodiments, certain fields can be pre-populated with recommended data values by a predictive process. For example, the stage/grade field 3808 A can be pre-populated with a stage field 3808 b (e.g., “advanced”).

Once suitable data values are added to the search parameter portion 3802, a search process can search a clinical trials database using the data values and display search results (e.g., clinical trials) in the search results portion 3810. In some embodiments, the search results can be filtered by a number of results filter fields 3812 such as a trial name filter field. In some embodiments, the GUI 3800 can be used to compare multiple clinical trials. A user can select multiple check boxes 3814 corresponding to a number of clinical trials and select a compare element 3816 (e.g., a compare button). In some embodiments, the search process can generate a relevance score 3818 for each clinical trial and/or rank the clinical trials by relevance score. The relevance score may be generated based on a number of factors including patient demographics as well as the location of the user. For example, clinical trials located closer to the user may be ranked higher than clinical trials located further away. In some embodiments, the relevance score 3818 can be formatted as yes/no, where yes indicated the patient is fit for the trial, and no indicates the patient is not fit for the trial.

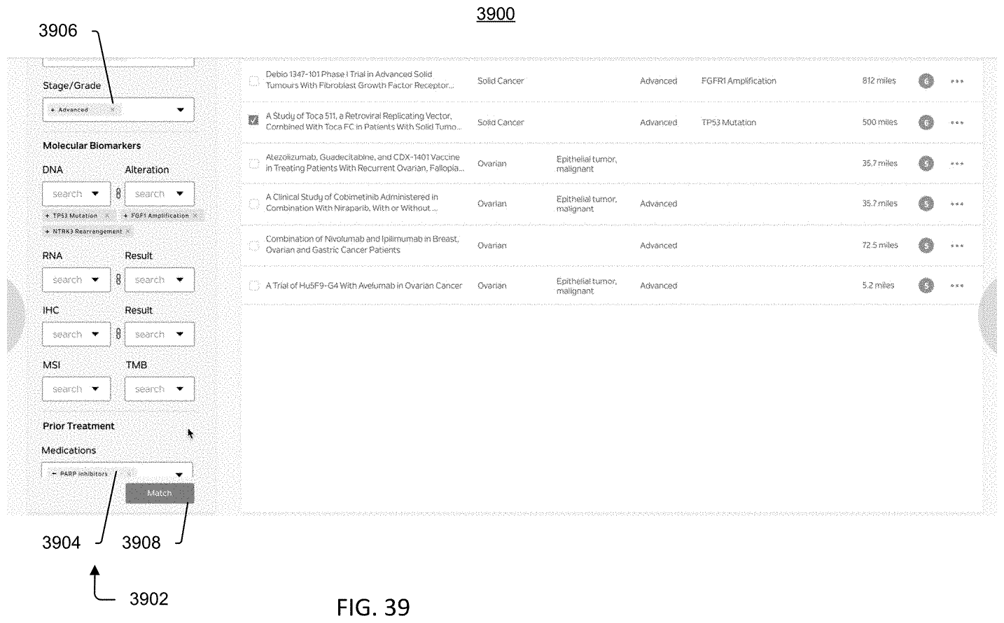

FIG. 39 is shown to include a graphical user interface (GUI) 3900. In some embodiments, GUI 3900 can be implemented by the system 100 in FIG. 1. In some embodiments, the GUI 3900 can include a search parameter portion 3902. The search parameter portion 3902 can include a number of data fields, each data field having a number of filters. The filters can include positive filters 3906 and negative filters 3904. The positive filters 3906 can indicate inclusion criteria, and the negative filters 3904 can indicate exclusion criteria. The search parameter portion 3902 can further include a match element 3908 (e.g., a match button) that can cause a search to be run by the search process.

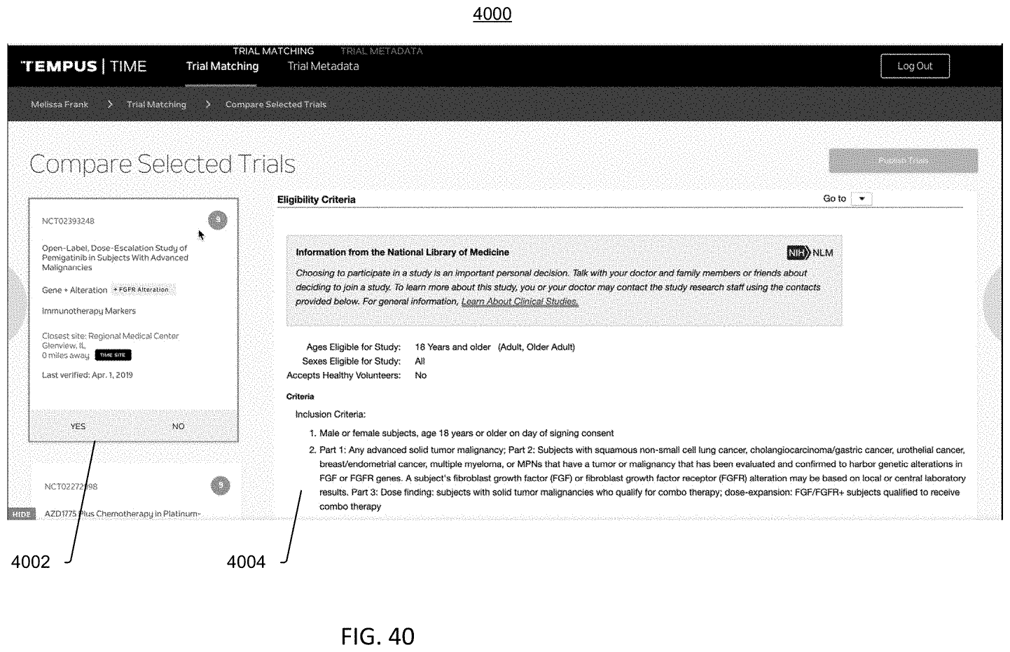

FIG. 40 is shown to include a graphical user interface (GUI) 4000. In some embodiments, GUI 4000 can be implemented by the system 100 in FIG. 1. In some embodiments, the GUI 4000 can show a selected clinical trial 4002 from search results and/or an information source 4004 (e.g., a website) that posted the clinical trial. In this way, a user can verify that the selected clinical trial 4002 identified in the search results is suitable for the patient. In some embodiments, the information source 4004 can be annotated with markers at locations and/or elements corresponding to the search parameters. In some embodiments, markers can be color coded (e.g., green for inclusion criteria, red for exclusion criteria), highlighted, and/or otherwise visually differentiated.

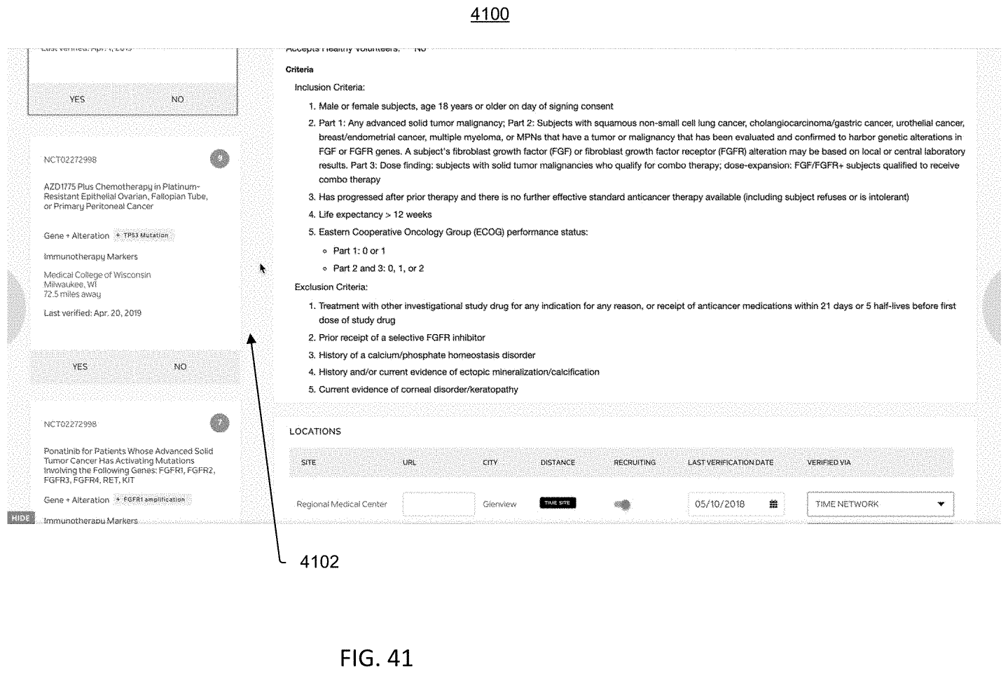

FIG. 41 is shown to include a graphical user interface (GUI) 4100. In some embodiments, GUI 4100 can be implemented by the system 100 in FIG. 1. In some embodiments, the GUI 4100 can show multiple clinical trials 4102 from the search results.

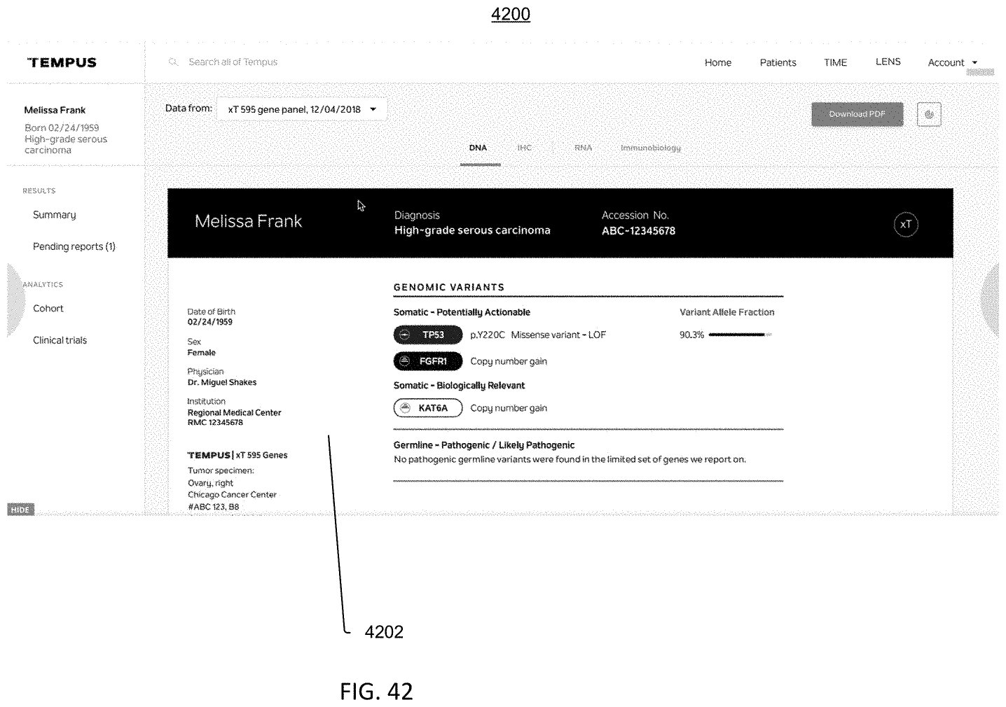

FIG. 42 is shown to include a graphical user interface (GUI) 4200. In some embodiments, GUI 4200 can be implemented by the system 100 in FIG. 1. In some embodiments, the GUI 4200 can include a patient report 4202 corresponding to a patient selected and/or applying for a clinical trial.

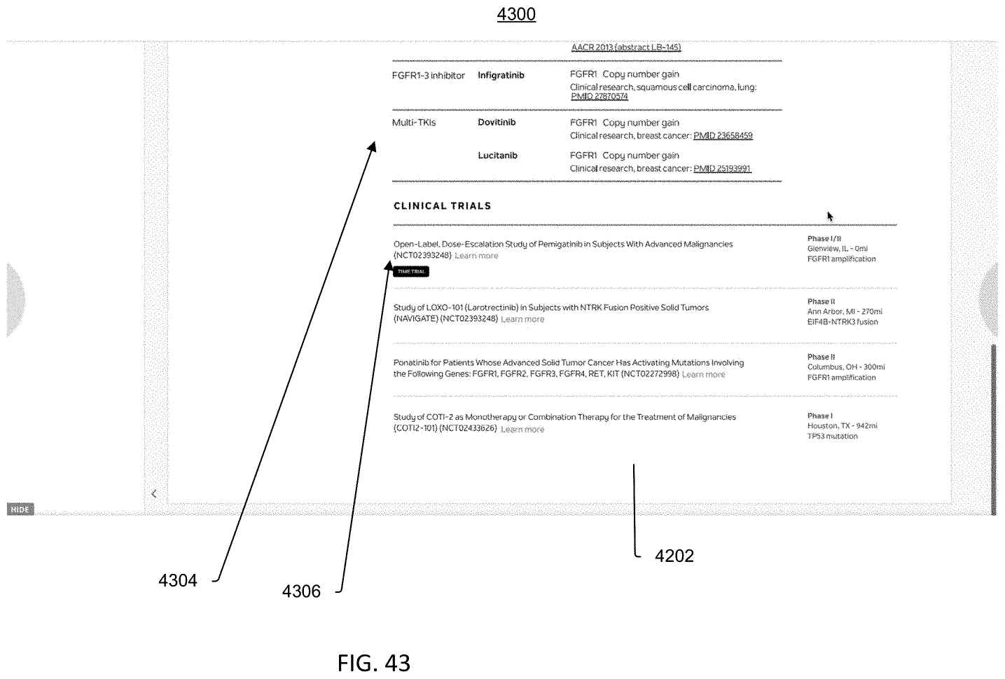

FIG. 43 is shown to include a graphical user interface (GUI) 4300. In some embodiments, GUI 4300 can be implemented by the system 100 in FIG. 1. In some embodiments, the GUI 4300 can include a patient report 4302 corresponding to a patient selected and/or applying for a clinical trial. The patient report 4302 can include a number of therapies 4304 (e.g., drug therapies) that have been matched to the patient based on DNA and/or RNA data. The patient report 4302 can include a number of clinical trials 4306 that have been matched to the patient.

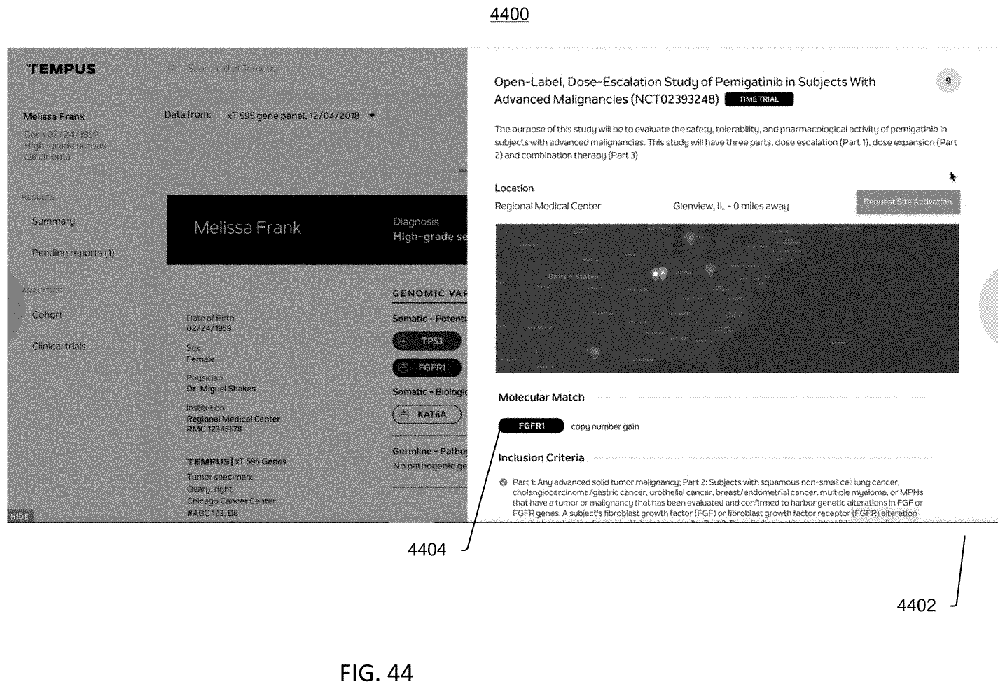

FIG. 44 is shown to include a graphical user interface (GUI) 4400. In some embodiments, GUI 4400 can be implemented by the system 100 in FIG. 1. In some embodiments, the GUI 4400 can include a clinical trial report 4402 including information about a selected clinical trial from the search results. In some embodiments, the selected clinical trial can be included in a final group (e.g., the top four scoring clinical trials) from the search results. The clinical trial report 4402 can include information about the clinical trial, such as a molecular match 4404 (e.g., a copy number gain required by the clinical trial that the patient possesses).

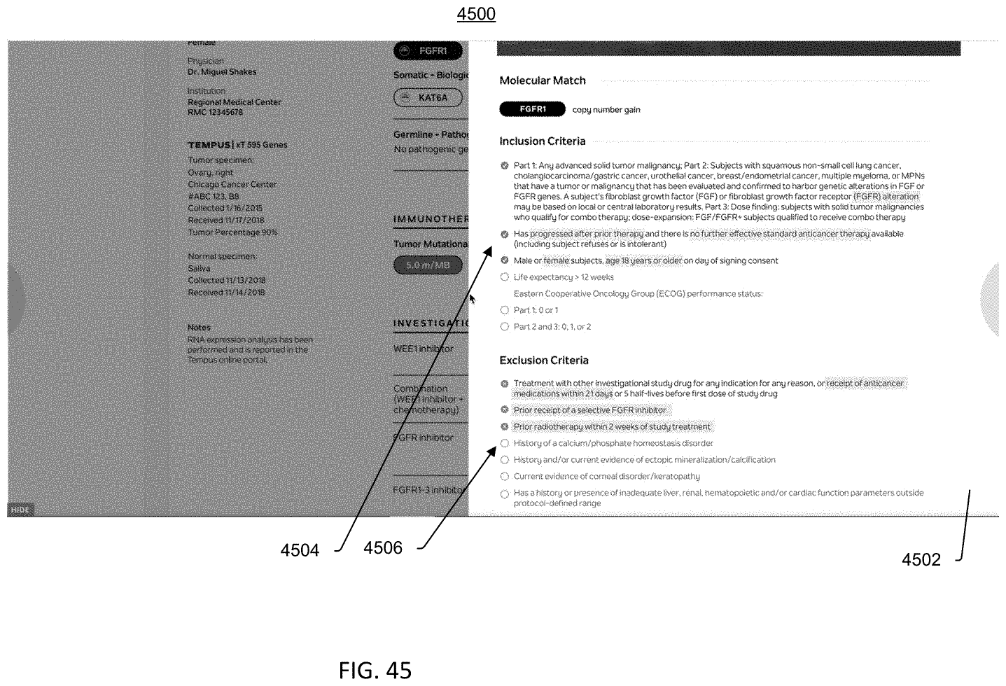

FIG. 45 is shown to include a graphical user interface (GUI) 4500. In some embodiments, GUI 4500 can be implemented by the system 100 in FIG. 1. In some embodiments, the GUI 4500 can include a clinical trial report 4502 including information about a selected clinical trial from the search results. In some embodiments, the selected clinical trial can be included in a final group (e.g., the top four scoring clinical trials) from the search results. The clinical trial report 4502 can include information about the clinical trial, such as inclusion criteria 4504 and/or exclusion criteria 4506. In some embodiments, the clinical trial report 4502 can include information about the clinical trial not included in a public source (e.g., a public website).

In some embodiments, the inclusion criteria 4504 can include excerpts taken directly from the original clinical trial source (e.g., clinicaltrials.gov). In some embodiments, portions of the excerpts included in the inclusion criteria 4504 can be highlighted (e.g., highlighted in green). The portions can be the portions of the original clinical trial source that were identified as inclusion criteria.

In some embodiments, the exclusion criteria 4506 can include excerpts taken directly from the original clinical trial source (e.g., clinicaltrials.gov). In some embodiments, portions of the excerpts included in the exclusion criteria 4506 can be highlighted (e.g., highlighted in red). The portions can be the portions of the original clinical trial source that were identified as exclusion criteria.

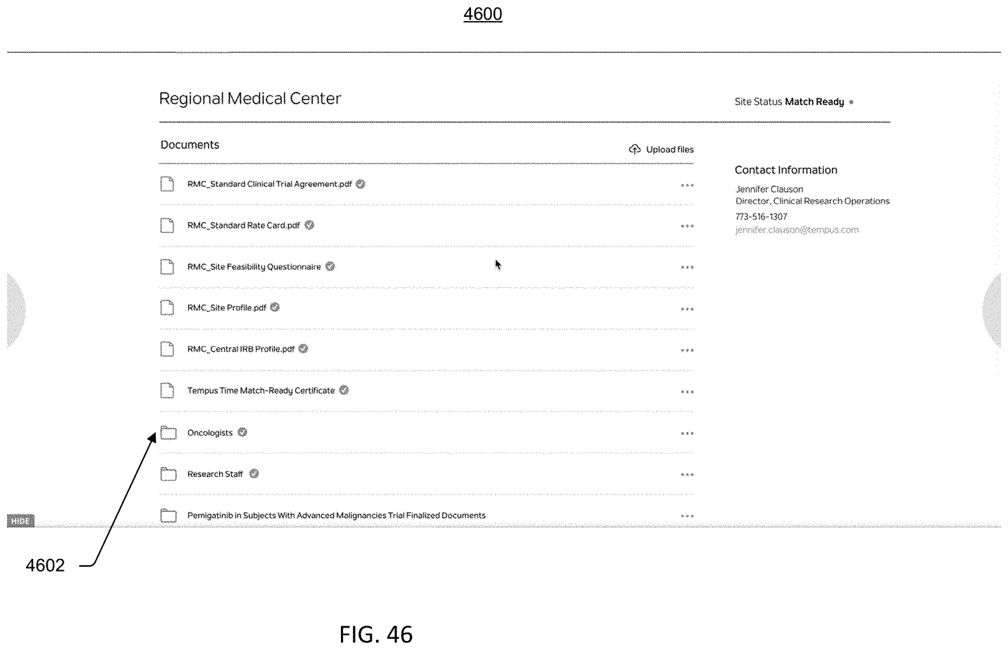

FIG. 46 is shown to include a graphical user interface (GUI) 4600. In some embodiments, GUI 4600 can be implemented by the system 100 in FIG. 1. In some embodiments, the GUI 4600 can include a number of documents and/or folders 4602 related to a clinical trial. At least some of the documents 4602 can be official documents (e.g., contracts) from the provider of the clinical trial. Some of the documents 4602 can include information about oncologists running and/or organizing the clinical trial.

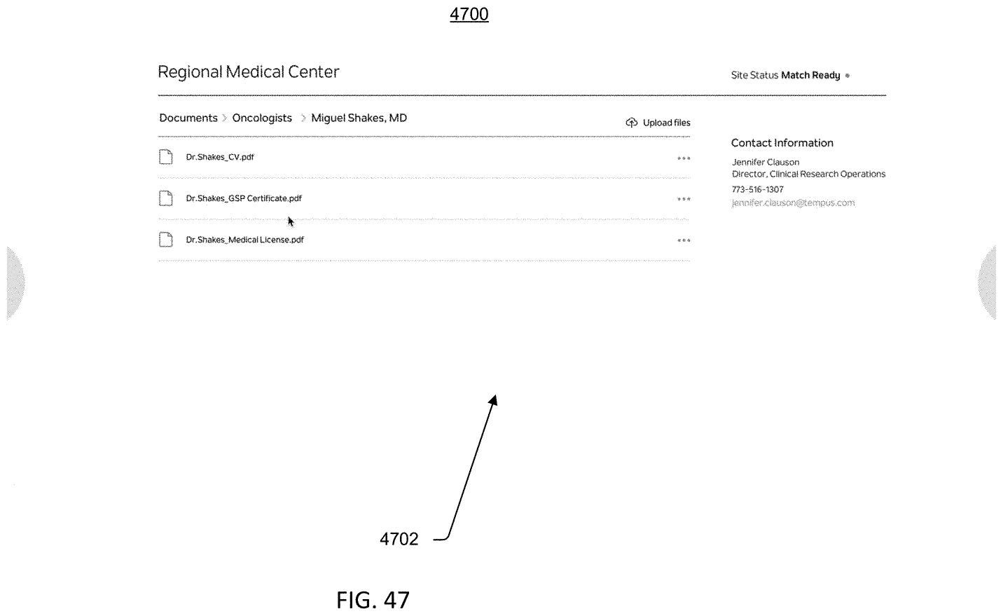

FIG. 47 is shown to include a graphical user interface (GUI) 4700. In some embodiments, GUI 4700 can be implemented by the system 100 in FIG. 1. In some embodiments, the GUI 4700 can include a number of documents 4702 related to an oncologist running and/or organizing the clinical trial. For example, the documents 4702 can include a CV and/or resume document, a certificate, a medical license, and/or other relevant documents.

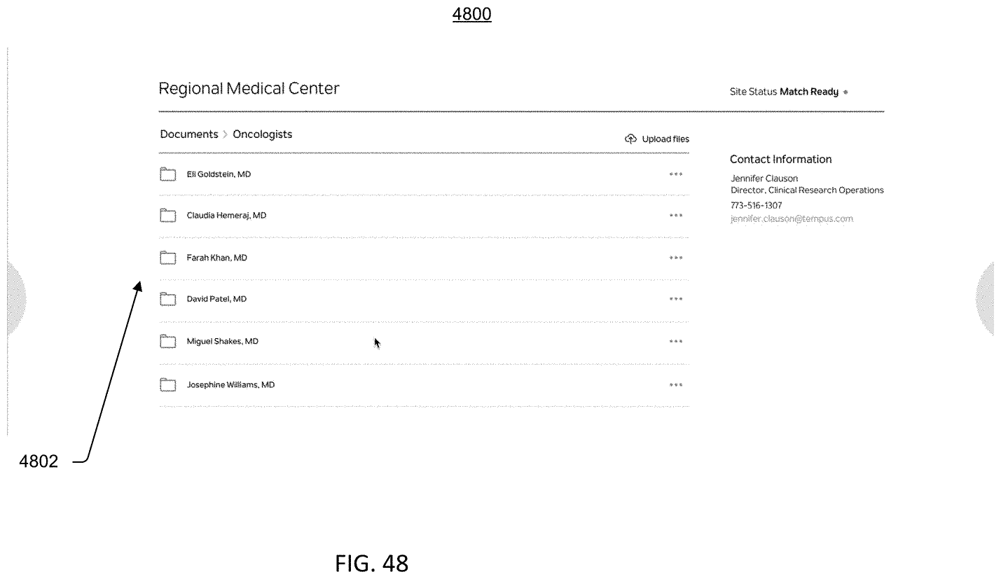

FIG. 48 is shown to include a graphical user interface (GUI) 4800. In some embodiments, GUI 4800 can be implemented by the system 100 in FIG. 1. In some embodiments, the GUI 4800 can include a number of folders 4802 related to oncologists running and/or organizing the clinical trial. Each of the folders 4802 can include a number of documents related to each oncologist (e.g., documents 4702 in FIG. 47).

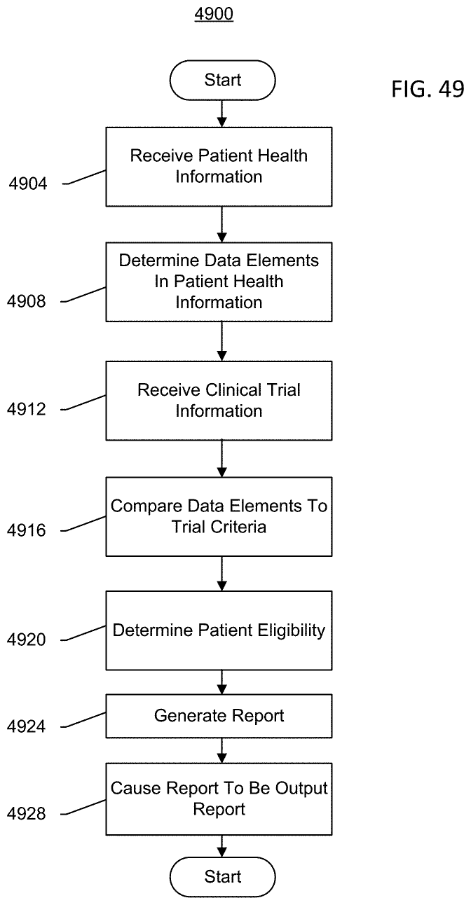

FIG. 49 is an exemplary process 4900 for determining patient eligibility for a clinical trial. In some embodiments, the process 4900 can be implemented as executable computer-readable instructions and stored on a non-transitory medium such as a memory. In some embodiments, the process 4900 can be executed by the system 100 in FIG. 1.

At 4904, the process 4900 can receive patient health information. In some embodiments, the patient health information can include information from an electronic medical record. In some embodiments, the patient health information can include at least a portion of the features in the patient data store 3202 in FIG. 32. In some embodiments, the patient health information can be unstructured.

At 4908, the process 4900 can determine data elements in the patient health information. In some embodiments, the patient health information can be unstructured and/or include free-text. The process 4900 can determine the data elements in order to standardize the patient health information. In some embodiments, the data elements can include at least a portion of the features and/or other data elements in the patient data store 3202.

At 4912, the process 4900 can receive clinical trial information. In some embodiments, the clinical trial information can include inclusion criteria and/or exclusion criteria. In some embodiments, the clinical trial information can include at least a portion of the information included in the inclusion and exclusion criteria module 3272 (e.g., inclusion criteria, exclusion criteria, clinical trial information, etc.). In some embodiments, the clinical trial information can include information about at least one clinical trial.

At 4916, the process 4900 can compare the data elements clinical trial information. In some embodiments, the process 4900 can compare at least a portion of the data elements to the inclusion criteria and/or at least a portion of the data elements to the exclusion criteria for each clinical trial. In some embodiments, the process 4900 can compare a molecular marker of the patient to the inclusion criteria and/or the exclusion criteria.

At 4920, the process 4900 can determine the eligibility of the patient for each of the at least one clinical trial. In some embodiments, the process 4900 can determine that the patient is eligible for each trial for which the patient does not meet any of the exclusion criteria and does meet at least a portion of the inclusion criteria. In some embodiments, the process may require that the patient meets at least a threshold amount (e.g., 60%) of the inclusion criteria to be eligible for a given clinical trial. The process 4900 can then determine any number of the at least one clinical trial for which the patient is eligible. The trials the patient is eligible for can be referred to as the at least one eligible clinical trial.

At 4924, the process can ge 4900 nerate a report for the patient. In some embodiments, the process 4900 can generate the report based on the at least one eligible clinical trial, the clinical trial information, and/or the patient health information. In some embodiments, the report can include at least a portion of the GUIs described above. For example, the report can include at least a portion of the GUIS 4200-4500.

At 4928, the process 4900 can cause the report to be output to at least one of a memory and/or a display (e.g., for viewing by a provider).

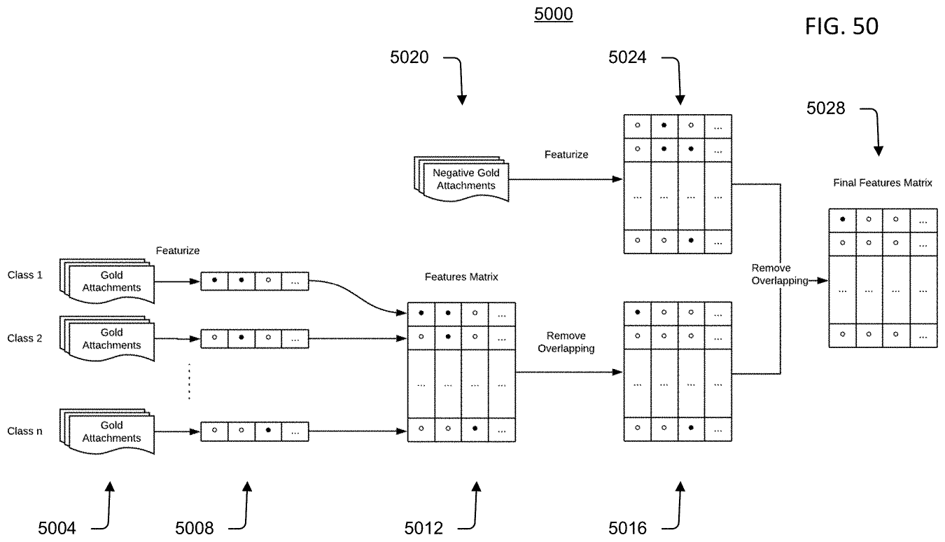

Referring now to FIG. 50, an exemplary flow 5000 for determining whether or not a next-generation sequencing (NGS) report is included in a medical report associated with a patient. In some embodiments, the flow 5000 can be implemented as one or more processes and/or executed by the system 100 in FIG. 1. In some embodiments, to predict the presence of molecular reports in a patient's case, the flow 5000 can generate a most probable label (e.g., a preparing organization name and/a or negative for the cases where no reports were predicted) based on the text of each document in the case.

In some embodiments, the flow 5000 can include computing the similarity between a bag of features of each document with the bag of features of a set of gold documents annotated for classification. In some embodiments, the flow 5000 can estimate report type for multiple organizations and/or report types. For example, in some embodiments, the flow 5000 can estimate report types for a number of organizations (e.g., organizations A-I) and a number of different test types as shown in Table 13 below:

TABLE 13

| Performing Organization | Report Name |
|---|---|
| Organization A | Test A1 - Heme |
| | Test A2 - CDx |
| Organization B | Test B1 - EGFR Mutation Analysis |
| | Test B2 - Microsatellite Instability |
| | Test B3 - KRAS Mutation Analysis |
| | Test B4 - NTRK NGS Fusion Profile |
| Organization C | Test C1 - Biopsy |
| Organization D | Test D1 - Molecular Intelligence |
| | Test D2 - Biomarker Analysis |
| | Test D3 - Tumor Profiling Assay |
| Organization E | Test E1 - BRAC Analysis |
| | Test E2 - Multi Gene/Multi Cancer |
| Organization F | Test F1 - Breast Cancer |
| Organization G | Test G1 - Early Stage Breast Cancer |
| Organization H | Test H1 - Liquid Biopsy |
| Organization I | Test I1 - SOLID TUMORS PANEL |
| | Test I2 - GLIOMA PANEL |
| | Test I3 - LUNG CANCER PANEL |
| | Test I4 - COLORECTAL CANCER PANEL |
| | Test I5 - MELANOMA PANEL |
| | Test I6 - MYELOID MALIGNANCIES |
| | Test I7 - MPN PANEL |
| | Test I8 - MPN DIAGNOSTIC PANEL |

Gold labels of gold documents 5004 can contain a diverse set of results including reports with “No alterations”, “No mutation”, “Instability not detected”, “Negative”, and/or “Positive” results. Some scans may not be of high quality and potentially affect optical character recognition (OCR) results. The flow 5000 can be robust enough to process reports even with lower quality scans. In some embodiments, the flow 5000 may not differentiate between negative results and positive results in generating predicted classifications.

### Preprocessing and Featurization

In some embodiments, the flow 5000 can include preprocessing the text of each page of a document by removing any duplicate consecutive characters and breaking any wrongly combined words into single words, which may be caused by an OCR technique. The flow 5000 can also include removing any short tokens, stop words, digits, punctuation tokens, and other tokens that look like numbers (e.g., ten, 3.9, etc.). In some embodiments, the preprocessing can inlcude using a spaCy/ScispaCy parser to parse text. After preprocessing, the flow 5000 can include extracting features 5008 such as emails, phone numbers, URLs, noun chunks, and unigrams from the preprocessed document's texts.

### Vectorization

The flow 5000 can include vectorising the extracted features 5008 per org/patient and forming a features matrix 5012. The flow 5000 can include pruning features matrix 5012 (e.g., to keep only the features or words which are unique per organization report) for a more accurate similarityrelevance calculation at the time of classification and form a filtered features matrix 5016. The flow 5000 can include further filtering the filtered features matrix 5016 to generate a final features matrix 5028. The flow 5000 can include filtering the filtered features matrix 5016 using the negative examples per class. The flow 5000 can include filtering the filtered features matrix 5016 to filter out the overlapping features with a feature vector 5024 generated based on negative gold documents 5020 (e.g., documents that have overlapping features with a certain class but are not a report, so removing the documents would improve precision) in order to generate the final features matrix 5028. The final features matrix 5028 can include a number of vectors associated with each of the gold documents 5004. Prediction

The flow 5000 can include predicting a classification (e.g., an organization) associated with a group of patient documents. At the time of prediction, the flow 5000 can generate a final features matrix 5028 as described above using the features documents. The flow 5000 can include generating a single class prediction per patient and/or a label per document (where one document can have multiple labels).

In some embodiments, the flow 5000 can include preprocessing, vectorizing, and pruning the vector for each documeant as mentioned above. The flow 5000 can include calculating a cosine similarity between the vector of the document and a matrix of organizations. The matrix of organizations can be a matrix that includes a number of vectors corresponding to a number of different test types and/or organizations.

In some embodiments, the flow 5000 can predict patient-level classifications for the patient documents.

For patient-level classification, the flow 5000 can include accumulating the similarities per document using a linear sum of the similarities, which can gather evidence per organization per document. The flow 5000 can include comparing the similarity per document to a threshold in order to remove a potential compound effect of small similarities across the set of patient documents. The flow 5000 can then generate a negative classification (e.g., if the sum of all thresholded similarities were zero across all documents and pages) or the organization name predicted.

For patient-level classification, the flow 5000 can include comparing the similarity per document to a threshold and output the classes that remain at a document level. The flow 5000 can then generate a negative classification (e.g., if the sum of all thresholded similarities were zero across all documents and pages) or the organization name predicted.

As described herein, the present disclosure includes systems and methods to help a medical provider make clinical decisions based on a combination of molecular and clinical data, which may include comparing the molecular and clinical data of a patient to an aggregated data set of molecular and/or clinical data from multiple patients, a knowledge database (KDB) of clinico-genomic data, and/or a database of clinical trial information. Additionally, the present disclosure may be used to capture, ingest, cleanse, structure, and combine robust clinical data, detailed molecular data, and clinical trial information to determine the significance of correlations, to generate reports for physicians, recommend or discourage specific treatments for a patient (including clinical trial participation), bolster clinical research efforts, expand indications of use for treatments currently in market and clinical trials, and/or expedite federal or regulatory body approval of treatment compounds.

1. A method of matching a patient to a clinical trial, the method comprising:
receiving text-based criteria for the clinical trial, including a molecular marker;
associating at least a portion of the text-based criteria to one or more pre-defined data fields containing molecular marker information;
comparing a molecular marker of the patient to the one or more pre-defined data fields; and
generating a report for a provider, the report based on the comparison and including a match indication of the patient to the clinical trial.

2. The method of claim 1, wherein the molecular marker is an RNA sequence.

3. The method of claim 1, wherein the molecular marker is a DNA sequence.

4. The method of claim 1, wherein the one or more pre-defined data fields include inclusion criteria and exclusion criteria.

5. The method of claim 1 further comprising:
determining that the patient has not received a treatment related to the molecular marker of the patient; and
determining that the patient is eligible for at least one candidate clinical trial in response to determining that the patient has not received the treatment.

6. The method of claim 1, wherein at least a portion of the text based criteria is free-text.

7. A clinical trial matching system comprising at least one processor and at least one memory, the system configured to:
receive text-based criteria for a clinical trial, including a molecular marker;
associate at least a portion of the text-based criteria to one or more pre-defined data fields containing molecular marker information;
compare a molecular marker of a patient to the one or more pre-defined data fields; and
generate a report for a provider, the report based on the comparison and including a match indication of the patient to the clinical trial.

8. The system of claim 7, wherein the molecular marker is an RNA sequence.

9. The system of claim 7, wherein the molecular marker is a DNA sequence.

10. The system of claim 7, wherein the one or more pre-defined data fields include inclusion criteria and exclusion criteria.

11. The system of claim 7, wherein the system is further configured to:
determine that the patient has not received a treatment related to the molecular marker of the patient; and
determine that the patient is eligible for at least one candidate clinical trial in response to determining that the patient has not received the treatment.

12. The system of claim 7, wherein at least a portion of the text based criteria is free-text.

13. A method of matching a patient to a clinical trial, the method comprising:
receiving health information from an electronic medical record corresponding to the patient;
determining data elements within the health information using at least one of an optical character recognition (OCR) method and a natural language processing (NLP) method;
comparing the data elements to pre-determined trial criteria, including trial inclusion criteria and trial exclusion criteria;
determining at least one matching clinical trial, based on the comparing of the data elements to the predetermined trial criteria; and
notifying a practitioner associated with the patient of the at least one matching clinical trial.

14. The method of claim 13, wherein the pre-determined trial criteria is generated based on unstructured text.

15. The method of claim 13, wherein the pre-determined trial criteria is formatted in at least one standardized format in use by a medical institution.

16. The method of claim 13, wherein the data elements include at least one of a clinical feature, a molecular feature, an epigenome feature, a microbiome feature, an organoid feature, or an imaging feature.

17. The method of claim 13 further comprising periodically updating a clinical trial database comprising the at least one matching clinical trial and at least one non-matching trial.

18. The method of claim 13, wherein notifying the practitioner associated with the patient of the at least one matching clinical trial comprises causing a report to be displayed to the practitioner, the report comprising the locations of the at least one matching trial.

19. A clinical trial matching system comprising at least one processor and at least one memory, the system configured to:
receive health information from an electronic medical record corresponding to the patient;
determine data elements within the health information using at least one of an optical character recognition (OCR) method and a natural language processing (NLP) method;
compare the data elements to pre-determined trial criteria, including trial inclusion criteria and trial exclusion criteria;
determine at least one matching clinical trial, based on the comparing of the data elements to the predetermined trial criteria; and
notify a practitioner associated with the patient of the at least one matching clinical trial.

20. The system of claim 19, wherein the pre-determined trial criteria is generated based on unstructured text.

21. The system of claim 19, wherein the pre-determined trial criteria is formatted in at least one standardized format in use by a medical institution.

22. The system of claim 19, wherein the data elements include at least one of a clinical feature, a molecular feature, an epigenome feature, a microbiome feature, an organoid feature, or an imaging feature.

23. The system of claim 19, wherein the system is further configured to periodically update a clinical trial database comprising the at least one matching clinical trial and at least one non-matching trial.

24. The system of claim 19, wherein notifying the practitioner associated with the patient of the at least one matching clinical trial comprises causing a report to be displayed to the practitioner, the report comprising the locations of the at least one matching trial.
